(19) United States
(12) Patent Application Publication
(10) Pub. No.: US 2026/0167686 A1
(43) Pub. Date: Jun. 18, 2026

(54) RECOMBINANT CCN DOMAIN PROTEINS AND FUSION PROTEINS

(71) Applicant: OSLO UNIVERSITETSSYKEHUS HF, Nydalen (NO)

(72) Inventors: Håvard Attramadal, Hobol (NO); Ole Jørgen Kaasbøll, Oslo (NO)

(21) Appl. No.: 19/360,074

(22) Filed: Oct. 16, 2025

Related U.S. Application Data
(63) Continuation of application No. 17/440,576, now Pat. No. 12,606,602.

(30) Foreign Application Priority Data
Mar. 20, 2019 (EP) 19163970.7

(51) Int. Cl. C07K 14/47; A61K 38/00

(52) U.S. Cl. CPC C07K 14/4702; A61K 38/00; C07K 2319/30; C07K 2319/31

(57) ABSTRACT

The present invention relates to recombinant proteins having an amino acid sequence corresponding to or related to the thrombospondin type 1 repeat homology domain of a member of the CCN family proteins and the use thereof. Furthermore, the present invention relates to fusion proteins comprising an amino acid sequence corresponding to or related to the thrombospondin type 1 repeat homology domain of a member of the CCN family proteins combined with a fusion partner and optionally a linker region. Also, novel protease resistant Fc-fragments are disclosed herein.

### CROSS REFERENCE TO RELATED APPLICATIONS

This application is a continuation of U.S. patent application Ser. No. 17/440,576, filed Sep. 17, 2021, which in turn is a 35 U.S.C. § 371 national phase entry of International Application No. PCT/EP2020/057773, filed Mar. 20, 2020, which claims priority to European Patent Application No. 19163970.7, filed Mar. 20, 2019, the disclosures of which are hereby incorporated by reference in their entireties.

### INCORPORATION BY REFERENCE OF MATERIAL SUBMITTED ELECTRONICALLY

The application is being with a sequence listing in electronic format. The sequence listing is provided as a file entitled “56868A_SeqListing.xml”, created Oct. 14, 2025, which is 198,855 bytes in size. The subject matter of the sequence listing is incorporated by reference herein in its entirety.

### FIELD OF INVENTION

The present invention relates to recombinant proteins having an amino acid sequence corresponding to or related to the thrombospondin type 1 repeat homology domain (domain III) of a member of the CCN family proteins and the use thereof, including particularly such proteins which are truncated and/or comprise certain amino acid modifications. Furthermore, the present invention relates to fusion proteins comprising an amino acid sequence corresponding to or related to the thrombospondin type 1 repeat homology domain of a member of the CCN family proteins combined with a fusion partner optionally via a linker peptide. In particular, the fusion partner is a monomeric protein, and the fusion proteins are monomeric.

Also, novel protease resistant Fc-fragments are disclosed herein.

### BACKGROUND OF THE INVENTION

CCN proteins are a family of secreted glycoproteins. CCN was originally coined as an acronym derived from the first three identified members of the gene family; Cyr61, CTGF and NOV. However the acronym has recently been adapted to be short for Cellular Communication Network factors and ratified by the HUGO Gene Nomenclature Committee (Perbal B, Tweedie S and Bruford E, J Cell Commun Signal. 2019 September; 13(3):435). The proteins are often classified as matricellular proteins, associated with the extracellular matrix (ECM). The CCN proteins are not part of the scaffold function, organizing the cells into tissues, but are rather considered signaling proteins and can function as independent autocrine or paracrine signaling factors, or as modifiers of other extracellular signaling proteins. Together with a set of three Wnt-inducible signaling pathway proteins (WISP1/CCN4, WISP2/CCN5, and WISP3/CCN6), they comprise a family of six homologous cysteine-rich proteins in mammals that have been renamed CCN1-6.

The initial members of the CCN family share a modular structure, with an N-terminal peptide signal for secretion followed by four conserved domains. The first domain shows sequence homologies to insulin-like growth factor binding proteins (IGFBP) and is thus known as IGF-binding protein homology domain, although only having neglible affinity for IGF. The second domain is known as the von Willebrand factor type C repeat (VWC) homology domain often seen in extracellular matrix (ECM) proteins. The third domain is known as the thrombospondin type I repeat homology domain (TSP-1) which may be involved in the attachment of CCN proteins to integrins. The forth domain is a cysteine-rich, C-terminal repeat or cystine knot homology domain, a domain reported to bind heparin. The 5^{th} member of the CCN protein family, the WISP2 (Wnt1-inducible signaling pathway protein 2), also known as CCN5, is atypical in lacking the carboxyl-terminal cystine knot domain (domain 4). The TSP-1 homology domains of the CCN protein family share 34% amino acid sequence identity and 25% sequence similarity (as analyzed by ClustalOmega, see below for reference). Four-domain CCN proteins contain 38 conserved cysteines throughout the primary sequence except for CCN6 in which 4 cysteines of the VWC homology domain are not conserved among the other family members. Also, for CCN5 which lacks the carboxyl-terminal cystine knot homology domain, all cysteines of the IGFBP, VWC and TSP-1 homology domains are conserved compared with the other CCN family members.

A non-conserved, protease-sensitive central region, often referred to as the hinge region, bisects the proteins into two halves. The expression of CCN proteins is regulated at transcriptional, post-transcriptional and translational levels in response to changes in environmental stimuli.

Information on the domain organization of the CCN-protein family is found in e.g. Liu et al, 2017, Journal of Diabetes, 9, pp. 462-474.

At the cellular level, CCN proteins may have diverse regulatory roles at the interface of the extracellular matrix and the cell surface. CCN proteins can regulate cell adhesion, migration, proliferation, differentiation, apoptosis, survival, senescence and gene expression. By modulating one or more aspects of these cellular functions, in a cell type-specific manner, CCNs coordinate complex biological processes, including cardiovascular and skeletal development during embryogenesis, as well as inflammation, wound healing, and tissue injury and repairin the adult. Generally speaking, the 4-domain CCN 1-4 and CCN6 (particularly CCN1, CCN2 and CCN4) may exert pro-fibrotic activity, whereas CCN5, which comprises only the 3 domains I-II, has anti-fibrotic activity.

CCN proteins are also involved in a broad diversity of pathological conditions, such as organ failure due to progressive fibrosis, for example hepatic fibrosis and idiopathic pulmonary fibrosis, and in cancer invasion and metastasis. Reference is in this respect made to Jun and Lau, 2011, Nat. Rev. Drug. Discovery, 10(12), pp. 945-963. Again, generally speaking, the 4-domain CCN proteins, in particular CCN2 have been shown to be implicated in the mechanisms of various fibrotic diseases, whereas in preclinical disease models of such diseases it has conversely been shown that increased CCN5 levels may be beneficial.

In Kaasbell et al., J. Biol. Chem, 293:46, pp. 17953-17970, it is reported that connective tissue growth factor (CTGF), also known as CCN2, is synthesized and secreted as an inactive preproprotein that requires proteolytic cleavage to release biologically active CCN2, and that a homodimer of the C-terminal fragment comprised of domains III-IV represents the biologically fully active form of CCN2, and finally, that all major reported activities of CCN2 could be recapitulated by the homodimer of the C-terminal domains III-IV fragment. Activity assays reported by Kaasbell et al. revealed that neither unprocessed, full-length CCN2 nor the N-terminal fragment comprised of domains I-II were biologically active. Furthermore, it was found that proteolytic processing of full-length CCN2 by matrix metalloproteinase (MMP)-activities released its latent activity. Collectively, the findings reported by Kaasbell et al. imply that prepro-CCN2 is autoinhibited by N-terminal domains I and II. It was also found that the C-terminal domains III and IV fragment of CCN1 and CCN3 was sufficient for activation of rapid cell signaling and eliciting cell physiologic responses. However, to what extent endopeptidase cleavage of the hinge region of CCN1 and/or CCN3, or any other CCN protein family member, are required for release of biologic activity, is unknown.

It is known that CCN2 is highly expressed during development, in various pathological conditions that involves enhanced fibrogenesis and tissue fibrosis, and in several cancers (Jun and Lau, 2011, supra). The fact that CCN proteins are involved in a broad diversity of pathological conditions, are extracellular proteins mechanistically involved in the development of fibrosis, and display limited expression in healthy organisms, make them attractive therapeutic targets.

Jeong et al., 2016, J. American College of Cardiology, 67: 13, pp. 1557-1568, reports on a study examining the role of adeno-associated virus-mediated gene transfer of CCN5 to murine hearts after experimentally-induced pressure overload of the heart. The study concludes that CCN5 may reverse established cardiac fibrosis by inhibiting generation of and enhancing apoptosis of myofibroblasts in the myocardium, suggesting that CCN5 may provide a platform for developing anti-cardiac fibrosis therapies.

In US2008/0207489, a method for treating a smooth muscle proliferation-based disorder is disclosed, involving expression of CCN5 or administering the CCN5 protein to smooth muscle cells.

In EP 2 556 839, a composition is provided comprising a genetic carrier comprising a nucleotide sequence encoding full-length CCN5 or CCN2ΔCT, and its role in treatment of heart failure is suggested. CCN2ΔCT in EP 2 556 839 is defined as the amino acid sequence of CCN2 truncated after K251 (uniprot numbering).

Although overexpression of CCN5 has been reported in some experimental systems to result in an opposite phenotype to that of CCN2 overexpression (Jeong et al. supra, Yoon et al., J Mol Cell Cardiol, 49 (2), 294-33 Aug. 2010), direct antagonization of the four-domain CCN proteins by CCN5 has to the authors' knowledge not been reported. In particular, the structural basis for CCN5/WISP2-mediated antagonism of the other CCN family members, was unknown prior to the work presented in this invention.

The inventors have at an earlier stage shown that full-length-CCN2 (FL-CCN2) is a preproprotein, an inactive precursor, and that a fragment comprising domains III and IV appears to convey all biologically relevant activities of CCN2. To what extent CCN proteins in general are secreted as inactive preproproteins that require proteolytic activation remains unknown. Yet, the susceptibility of full-length CCN-proteins to multiple proteases, as demonstrated by the inventors (Kaasbell et al., J. Biol. Chem. (2018) 293(46) 17953-17970) and others (Butler, G. S. et al. Matrix Biol 59, 23-38 (2017) and Guillon-Munos, A. et al. J Biol Chem 286, 25505-25518 (2011)) implies that unmodified, full-length CCN proteins will be highly unsuitable as drugs for reasons of stability both during recombinant protein manufacturing and following in vivo administration. This unsuitability of using unmodified, full-length CCN proteins as therapeutic proteins also applies to fusion proteins of full-length CCN proteins, e.g. as described for full-length CCN1 (Schutze, N. et al. (2005) Protein Expr Purif 42, 219-225) and full-length CCN6 (Schutze, N et al. (2007) BMC Cell Biol 8, 45). It is well known in the field of CCN proteins that the susceptibility of these proteins to proteolysis is one of the reasons why it is very difficult to produce recombinant CCN proteins. Furthermore, based on the novel findings of Kaasbell et al. (J Biol Chem 2018; 293(46):17953-17970) recombinant full-length CCN proteins may be far from ideal biologic drugs, as their activity may be dependent on prior proteolytic processing, making pharmacokinetics and pharmacodynamics unpredictable. Furthermore, in the case of Fc-fusion proteins, in addition to the proteolytic susceptibility of the components, e.g. peptide linker, CCN-fragment and Fc-fragment, the arrangement of the components has also been shown to be of importance for the efficacy and potency of the recombinant fusion proteins. One example of this is in the paper published by the inventors (Kaasbell et al. (2018)), in which variants of Fc-fusion proteins containing domains III-IV of CCN2 are found to have widely varying activities in a manner not easily predictable beforehand.

The actions of CCN proteins has been reported to be susceptible to antagonizing effects by high concentrations of synthetic peptides derived from the primary sequences of the CCN proteins. One example is inhibition of AKT phosphorylation stimulated by recombinant CCN2 in Rat2 fibroblasts by peptides derived from domain I, the IGFBP homology domain, and to a lesser extent peptides derived from domain III, the TSP-1 repeat homology domain, of CCN2 (Moe et al., J. Cell Commun. Signal. (2013) 7:31-47). Another example is the inhibition of CCN2 (domain IV)-stimulated adhesion of hepatic stellate cells by a peptide derived from domain IV, the cystin knot homology domain, of CCN2 (Gao R and Brigstock D R., J Biol Chem. 2004 Mar. 5; 279(10):8848-55). Furthermore, peptides from domain III of CCN1 (Leu et al. J. Biol. Chem, 2003, Vol. 278, No. 36, Issue of September 5, pp. 33801-33808, 2003) and domain III of CCN1, CCN2, CCN3, CCN5 and CCN6 (Karagiannis E G and Popel The International Journal of Biochemistry & Cell Biology 39 (2007) 2314-2323) have been reported to have some anti-angiogenic effects in in vitro assays with HUVEC cells (Leu et al. J. Biol. Chem, 2003 and Karagiannis E G and Popel, Int J Biochem Cell Biol 39 (2007)) and anti-adhesion effects on 1064SK human foreskin fibroblasts (Leu et al. J. Biol. Chem, 2003), these peptides only contain one (Leu et al. J. Biol. Chem, 2003) or two (Karagiannis E G and Popel, Int J Biochem Cell Biol 39, 2007) of the conserved cysteines which are central to the invention described in this document. The cysteines in domain III of the CCN proteins are known to create disulfide bridges, as demonstrated in CCN2 endogenously expressed from HUVEC cells (Lu, S et al. (2015) Nat methods 12, 329-331) and from purified, recombinant CCN2 (Kaasbell et al., J. Biol. Chem. 2018). The disulfide bridges demonstrated in CCN2 spanning C199-C228 (uniprot numbering) confers a complex 3D structure where the amino acid chain folds back upon itself. This implies that the complete domain III of a CCN protein cannot be expected to be replicated by short peptides that are not structurally constrained by disulfide-bridges between the cysteines as in the complete domain III of the CCN proteins produced in eucaryotic systems. Furthermore, the inhibition of CCN2 activities by peptides derived from the primary sequences of domains I, II, and IV illustrates the lack of knowledge in the field regarding whether peptides derived from a specific domain of CCN2 can confer inhibition of four-domain CCN proteins.

The inventors have now, based on structure-activity analysis of CCN family proteins and the observation that CCN2 need to undergo proteolytic processing in order to become biologically active, found that the biologically active part of the CCN5 protein is domain III, the thrombospondin type I repeat homology domain. This new knowledge has resulted in the providing of bioactive structures based on domain III of CCN5 as well as domain III of other members of the CCN protein family.

### SUMMARY OF INVENTION

The present inventors insights into the structure-activity relationship of CCN5, and other CCN-proteins has resulted in the providing of novel biologically active recombinant proteins that recapitulate the cell signaling and cell physiological functions ascribed to CCN5-signaling and, which may also counteract the other four-domain CCN proteins (Cyr61 (also known as CCN1), CTGF (also known as CCN2), NOV (also known as CCN3), WISP1 (also known as CCN4) and WISP3 (also known as CCN6)). In other words, proteins are provided, including in the form of fusion proteins, based on domain III, the TSP-1 homology domain, of a CCN protein, which recapitulate, or which have, the biological activity of CCN5, and which are able to antagonize, or inhibit, the effects of the 4-domain CCN proteins, CCN1-4 or CCN6. In particular the proteins herein have anti-fibrotic activity and may also have direct anti-cancer activity.

As noted above, domain III (the TSP-1 homology domain) of the other CCN proteins, namely the 4-domain CCN proteins, when provided as a separate domain in the absence of the other CCN domains, has surprisingly been found to be sufficient to recapitulate the reported activities of CCN5. Accordingly, in other words, domain III of the 4-domain CCN proteins, when provided as a separate domain in the absence of the other CCN domains (i.e. as an isolated domain), has the same activity as CCN5, or, alternatively expressed, as domain 11/TSP-1 homology domain of CNN5, (that is an opposite activity to that of full-length 4-domain CCN proteins). Thus from the experiments disclosed in this document it is clear that the isolated TSP-1 homology domain of any CCN protein may exert the same activity as that of the TSP-1 homology domain of CCN5. Other than in the case of CCN5, this may not be the same as the activities exerted by the full-length CCN protein.

It has been found that monomeric fusion proteins, wherein the domain Ill of a CCN protein is fused to a monomeric fusion partner, are of particular benefit and utility according to the invention and disclosure herein.

According to a first aspect, the present invention provides a monomeric fusion protein comprising:
(i) a polypeptide corresponding to at least a portion of the thrombospondin type 1 repeat (TSP-1) homology domain of a CCN family protein;
(ii) a monomeric fusion partner N- or C-terminally fused to the amino acid sequence of (i); and
(iii) optionally a peptide linker between the polypeptide of (i) and the monomericfusion partner of (ii),
wherein the polypeptide of (i) is 40 to 60 amino acids in length and comprises an amino acid sequence selected from SEQ ID NOs: 37 or 2 to 6, or a sequence having at least 80% sequence identity to a sequence selected from SEQ ID NOs: 37 or 2 to 6 wherein all of the cysteine residues in said sequence selected from SEQ ID NOs: 37 or 2 to 6 are conserved, and wherein the monomeric fusion partner of (ii) and the peptide linker of (iii) are not or do not comprise an IGF binding protein homology domain, a von Willebrand factor type C repeat homology domain, or a cysteine knot domain of a CCN family protein.

As will be described in more detail below, SEQ ID NOs. 37, and 2-6 represent 44 amino acid truncated fragments of domain III of CCN5, CCN3, CCN2, CCN1, CCN4 and CCN6, respectively, which comprise the 6 conserved cysteine residues of this domain. In particular the fragments are flanked by the first and last cysteine residues of the domain. It has been found that such fragments are particularly effective and resistant to proteolytic degradation.

In an embodiment, the polypeptide of (i) comprises or consists of:
(a) an amino acid sequence selected from SEQ ID NOs: 1 or 8 to 12; or
(b) an amino acid sequence having at least 80% sequence identity to a sequence selected from SEQ ID NOs: 1 or 8 to 12; or
(c) a part of an amino acid sequence of (a) or (b), wherein said part comprises at least the 44 amino acid sequence of SEQ ID NOs: 37, 6, 2, 3, 4 or 5 respectively or a sequence having at least 80% sequence identity to a sequence selected from SEQ ID NOs: 37, 6, 2, 3, 4 or 5 respectively.

SEQ ID NOs. 1, and 8-12 represent slightly longer N-terminally truncated fragments of domain III of CCN5, CCN6, CCN3, CCN2, CCN1, and CCN4 respectively. These fragments comprise the corresponding sequences of SEQ ID NOs. 37, 6, 2, 3, 4, and 5 respectively, with some additional C-terminal sequence from the respective domain III.

In a further embodiment, the polypeptide of (i) comprises an alanine residue at the position corresponding to position 2 of said sequence selected from SEQ ID NOs: 37 or 2 to 6, or SEQ ID NOs: 1 or 8 to 12. In some embodiments, the amino acid sequence of (i) comprises an amino acid sequence selected from SEQ ID NO: 38 or 42 to 46, or an amino acid sequence having at least 80% sequence identity thereto. In another embodiment, the amino acid sequence of (i) comprises an amino acid sequence selected from SEQ ID NO: 7, or 47 to 51, or an amino acid sequence having at least 80% sequence identity thereto. It has been found in this respect that substitution of this residue at position 2 is beneficial in promoting stability of the protein.

According to a further aspect of the invention, the monomeric fusion protein has an amino acid sequence selected from the group consisting of SEQ ID NOs: 84, 85, 88, 89, 97, 98, 102, 103, 106, 107, 110, 111, or an amino acid sequence having 80% sequence identity thereto.

In embodiments of these aspects the monomeric fusion partner is selected from serum albumin, transferrin and monomeric Fc fragments, particularly monomeric Fc fragments of IgG, more particularly human IgG.

As noted above, substitution of position 2 of the domain III fragments herein improves the stability of the fragment, in particular resistance to protease degradation. It is believed and proposed herein that such sequence-modified variants of the domain III fragments represent useful proteins in their own right, without being linked to a fusion partner.

Accordingly, another aspect of the invention also provides a protein 40 to 60 amino acids in length which comprises, or a protein which consists of, an amino acid sequence as set out in SEQ ID NO: 7, 38, 42 to 46, or 47 to 51 or a sequence with at least 80% sequence identity thereto, wherein the protein comprises an alanine residue at the position corresponding to position 2 of said sequence of SEQ ID NO: 7, 38, 42 to 46, 47 to 51, and wherein all of the cysteine residues in said sequence are conserved.

Other proteins and fusion proteins are also provided as further aspects of the inventions, as detailed below.

According to a further aspect of the invention, a recombinant protein is provided comprising a of formula (I)

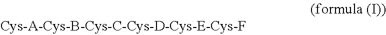

wherein
A is a peptide of formula II

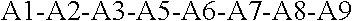

wherein A1 is selected from the group consisting of P, A, V, I, and L; A2 is selected from the group consisting of E, D, A, I, L, and V; A3 is selected from the group consisting of G, Q, Y, S, N, W, F; A4 is selected from the group consisting of A, I, L, V, S, T; A5 is an amino acid selected from the group consisting of T, Y, N, G, Q and S; A6 is an amino acid selected from the group consisting of A, V, I, L, P, S, E, D, K, R, and H; A7 is W; A8 is selected from the group consisting of G, T, S, Q, Y, N, P, A, V, I, and L; A9 is an amino acid selected from the group consisting of A, P, L, I, V, Q; and
B is a peptide of formula III

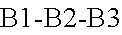

wherein B1 is an amino acid selected from the group consisting of G, Q, N, S, Y, and T; B2 is an amino acid selected from the group consisting of, T, S, N, F, Q, H, R and K; B3 is an amino acid selected from the group consisting of G, Q, N, S, Y, T; wherein one of B1-B3 is absent; and
C is a peptide of formula IV

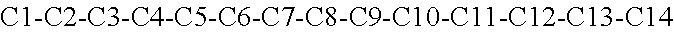

Wherein C1 is an amino acid selected from the group consisting of G, Q, N, S, Y, and T; C2 is an amino acid selected from the group consisting of K, R, H, M, T, S, A, L, I, and V; C3 is an amino acid selected from the group consisting of G, Q, N, S, Y, and T; C4 is an amino acid selected from the group consisting of M, F, A, I, L, V, and W; C5 is an amino acid selected from the group consisting of G, Q, N, S, T, Y, A, I, L, and V; C6 is an amino acid selected from the group consisting of G, Q, N, S, and T; C7 is an amino acid selected from the group consisting of H, R, and L; C8 is an amino acid selected from the group consisting of A, L, I, and V; C9 is an amino acid selected from the group consisting of G, Q, N, S, T and Y; C10 is an amino acid selected from the group consisting of G, Q, N, S, T, Y (preferably N); C11 is an amino acid selected from the group consisting of V, P, A, I, L, G, Q, N, S, T, Y, R, K, D, and E; C12 is an amino acid selected from the group consisting of G, Q, N, S, Y, and T; C13 is an amino acid selected from the group consisting of H, K, R, A, L, I, V, P, G, Q, N, S, Y, and T; C14 is an amino acid selected from the group consisting of F, P, W, G, Q, N, S, Y, T, E, and D; and
D is a peptide of formula VD1—

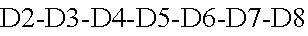

wherein D1 is an amino acid selected from the group consisting of R, K, H, D, E, W, P; D2 is an amino acid selected from the group consisting of P, A, L, I, V, M, W, D, and E; D3 is an amino acid selected from the group consisting of D, E, A, L, I, V, R, K, and H; D4 is an amino acid selected from the group consisting of G, Q, S, Y, T, R, L, K, and H; D5 is an amino acid selected from the group consisting of G, Q, N, S, Y, T, D, and E; D6 is an amino acid selected from the group consisting of H, R; K, G, Q, N, S, Y, and T; D7 is an amino acid selected from the group consisting of L, H, and R; D8 is an amino acid selected from the group consisting of A, L, I, and V; and
E is a peptide of formula VI

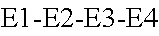

wherein E1 is an amino acid selected from the group consisting of P, A, L, I, V, M, W, G, Q, N, S, T, Y, D, and E; E2 is an amino acid selected from the group consisting of; P, A, L, I, V, M, W, G, Q, N, S, T, Y; E3 is an amino acid selected from the group consisting of, R, K, H, G, Q, N, S, T and Y; E4 is an amino acid selected from the group consisting of P, A, L, I and V; F is absent or an amino acid sequence of up to about 13 amino acids,
wherein the recombinant protein comprises from 40 to 60 amino acid.

According to one embodiment of the above aspect, a recombinant protein of formula (I) is provided, wherein
A1 is selected from the group consisting of P, I, and L; A2 is selected from the group consisting of E, V, and A; A3 is selected from the group consisting of W, Q, and Y; A4 is selected from the group consisting of S, T, and A; A5 is an amino acid selected from the group consisting of T and S; A6 is an amino acid selected from the group consisting of A, E, P, S and K; A7 is W; A8 is selected from the group consisting of G, S and T; A9 is an amino acid selected from the group consisting of P, Q and A; and
B1 is serine (S); B2 is an amino acid selected from the group consisting of T, K and R; B3 is an amino acid selected from the group consisting of T and S; and
C1 is an amino acid G; C2 is an amino acid selected from the group consisting of T, L and M; C3 is G; C4 is an amino acid selected from the group consisting of M, F, I, and V; C5 is an amino acid selected from the group consisting of S and A; C6 is an amino acid selected from the group consisting of T and N; C7 is R; C8 is an amino acid selected from the group consisting of V, and I; C9 is an amino acid selected from the group consisting of S, and T; C10 is asparagine N; C11 is an amino acid selected from the group consisting of Q, R, D, V, and E; C12 is asparagine N; C13 is an amino acid selected from the group consisting of R, A, P, and S; C14 is an amino acid selected from the group consisting of F, Q, S, E, and N; and
D1 is an amino acid selected from the group consisting of R, E, and W; D2 is an amino acid selected from the group consisting of L, M, and P; D3 is an amino acid selected from the group consisting of E, L, V and R; D4 is an amino acid selected from the group consisting of T, K, and Q; D5 is an amino acid selected from the group consisting of Q and E; D6 is an amino acid selected from the group consisting of R, T, S, and K; D7 is arginine (R); D8 is an amino acid selected from the group consisting of L, and I; and
E1 is an amino acid selected from the group consisting of L, M, E, N, and Y; E2 is an amino acid selected from the group consisting of; S, V, L and I; E3 is an amino acid selected from the group consisting of, Q and R; E4 is P; F is absent or a peptide of up to 13 amino acids and comprising an amino acid sequence selected from the group consisting of PPSRGRSPQNSAF, GQPVYSSL, EADLEEN, EQEPEQPTD, DVDIHTLI, and DSNILKTIKIP,
wherein the recombinant protein comprises in total from 44 to 57 amino acid.

According to yet an embodiment of the above aspect, a recombinant protein of formula I is provided wherein F is fully absent, partially absent, or a peptide of about 13 amino acids comprising the amino acid sequence of PPSRGRSPQNSAF.

More particularly, a recombinant protein is provided, wherein the protein comprises an amino acid sequence selected from the group consisting of SEQ ID No. 1, SEQ ID No. 2, SEQ ID No. 3, SEQ ID No. 4, SEQ ID No. 5, SEQ ID No. 6, and SEQ ID No. 7, SEQ ID No.8, SEQ ID No. 9, SEQ ID No. 10, SEQ ID No. 11, SEQ ID No. 12, SEQ ID No. 37, SEQ ID No. 38; and fragments or variants thereof having above 50% sequence identity with the amino acid sequences SEQ ID No. 1, SEQ ID No. 2, SEQ ID No. 3, SEQ ID No. 4, SEQ ID No. 5, SEQ ID No. 6, and SEQ ID No. 7, SEQ ID No.8, SEQ ID No. 9, SEQ ID No. 10, SEQ ID No. 11, SEQ ID No. 12, SEQ ID No. 37, SEQ ID No. 38.

According to another aspect of the invention, recombinant proteins as defined above are provided, wherein said protein is pegylated.

According to another aspect, the present invention provides a fusion protein comprising
(i) Thrombospondin type 1 repeat (TSP-1) homology domain of a CCN family protein;
(ii) fusion partner N- or C terminally fused to the TSP-1 repeat homology domain of (i) and wherein said fusion partner is selected from the group consisting of serum albumin, transferrin and Fc-fragment of human IgG;
(iii) optionally a peptide linker between the TSP-1 repeat homology domain and the Fc fragment (N- or C-terminally fused to the TSP-1 repeat homology domain of (i)).

According to an embodiment of the above aspect, fusion proteins are provided comprising a recombinant protein according to the present invention as described above as a further aspect of the invention.

The fusion partner of a fusion protein according to the invention is according to one embodiment selected from the group consisting of an Fc fragment of IgG1, IgG2 or IgG4, serum albumin and transferrin.

According to a further embodiment, a fusion protein is provided wherein the fusion partner (ii) is an Fc fragment of IgG1, IgG2 or IgG4 comprising a stabilizing disulfide bridge. Such mutations may increase thermal stability of the protein. Stabilising mutations are known and have been reported in the art.

According to yet a further embodiment, a fusion protein is provided wherein the fusion partner (ii) is an Fc fragment of IgG1, IgG2 or IgG4 comprising one or more mutations selected from the group consisting of S228P (refers to IgG4), E233P (refers to IgG1 and IgG4), F234A (refers to IgG4), L234A (refers to IgG1), L234V (refers to IgG1), F234V (refers to IgG4), L235A (refers to IgG1 and IgG4), ΔG236 (refers to IgG1 and IgG4) and ΔK447 (refers to IgG1, IgG2 and IgG4).

According to another embodiment, a fusion protein may comprise an Fc fragment selected from the group consisting of SEQ ID No. 15, SEQ ID No. 16, SEQ ID No. 17, SEQ ID No. 18, and SEQ ID No. 19.

According to another embodiment, a fusion protein comprises a linker selected from the group consisting of SEQ ID No. 20; SEQ ID No. 21; SEQ ID No. 22, SEQ ID No. 23, SEQ ID No. 24, SEQ ID No. 25, and SEQ ID No. 39.

According to one embodiment, the linker comprises the amino acid sequence (EAAAK)n, wherein n is at least 4, preferably n is 8.

According to another embodiment, the fusion protein comprises an amino acid sequence selected from the group consisting of SEQ ID No. 26, SEQ ID No. 27; SEQ ID No. 28, SEQ ID No. 29, SEQ ID No. 30, SEQ ID No. 31, SEQ ID No. 40 and SEQ ID No. 41.

According to another embodiment, the fusion partner of (ii) is serum albumin.

According to another embodiment of the second aspect of the invention, the fusion partner of (ii) is transferrin.

The present invention furthermore, according to a still further aspect, provides a nucleic acid molecule (e.g. DNA) encoding a recombinant protein, protein or fusion protein according to the present invention.

According to one embodiment of this aspect, a DNA sequence is provided comprising a nucleic acid sequence as depicted in SEQ ID No. 34, SEQ ID No. 35, SEQ ID No. 36 or SEQ ID NOs: 86, 87, 90, 91, 99, 100, 104, 105, 108, 109, 112 or 113 and nucleic acid sequences having about 80% sequence identity with SEQ ID NO. 34, SEQ ID No. 35, SEQ ID No. 36 or SEQ ID NOs: 86, 87, 90, 91, 99, 100, 104, 105, 108, 109, 112 or 113.

Furthermore, according to another aspect of the invention, an expression vector is provided comprising a DNA sequence according to the present invention. Also a host cell comprising an expression vector according to the invention is provided.

Finally, a thrombospondin type 1 repeat (TSP-1) homology domain of a CCN family protein, a protein and a fusion protein as defined above is provided for use as a medicament is provided for treatment or prevention of disorders by inhibiting or counteracting the cell signaling, and cell physiological functions ascribed to the four-domain CCN family proteins.

In one aspect, there is provided a fusion protein, or a protein, as defined herein for use in therapy.

The fusion protein or protein may be for use in the treatment of a condition associated with activity of a 4-domain CCN protein, particularly unwanted or abberant activity of a 4-domain CCN protein. The activity may be associated with a fibrotic effect. The activity may be pro-fibrotic activity.

In another aspect, there is provided a fusion protein, or a protein, as defined herein for use in the treatment or prevention of fibrosis, or any condition exhibiting fibrosis (i.e. a fibrotic condition or disease). In a further aspect, there is provided a fusion protein, or a protein, as defined herein for use in the treatment of cancer. Also provided is a fusion protein, or a protein, as defined herein for use in the treatment of inflammatory or autoimmune diseases, or metabolic diseases.

Also provided according to such aspects of the invention is the use of a protein or a fusion protein as defined herein for the manufacture of a medicament for treating or preventing a condition or disease as defined or described herein.

Such aspects also include a composition (e.g. a pharmaceutical composition) comprising a protein or a fusion protein as defined herein for use in treating or preventing a condition or disease as defined or described herein.

Such aspects also include a method of treating or preventing a condition or disease as defined or described herein, said method comprising administering to a subject in need thereof a protein or a fusion protein as defined herein, particularly an effective amount of a said protein or fusion protein.

### FIGURES

FIG. 1 shows the cell physiological and cell signaling of CCN5(dIII)-Fcv2 (an embodiment of the invention as defined in sequence SEQ ID No. 28).
A) shows that CCN5(dIII)-Fcv2 fusion protein of SEQ ID No. 28 causes concentration-dependent inhibition of phosphorylation of Akt (Serine-473) in A549 lung cancer cells.
B) Shows that the CCN5(dIII)-Fcv2 fusion protein of SEQ ID. No. 28 inhibits proliferation in a human lung fibroblast cell line, IMR90.
C) Shows that the CCN5(dIII)-Fcv2 fusion protein of SEQ ID No. 28 inhibits the sphere-forming ability (anchorage-independent growth) of the estrogen receptor-positive breast cancer cell line MCF-7 and of the triple-negative breast cancer cell line MDA-MB-231.
D) Shows that the CCN5(dIII)-Fcv2 fusion protein of SEQ ID No. 28 dose-dependently inhibits TGF-β-induced SMAD reporter activity (SMAD proteins are canonical TGF-β-regulated transcription factors).

All error bars show SD. Statistical significance calculated by 1-way ANOVA with Dunnetts post hoc test (p<0.05 indicated by *).

FIG. 2 shows the effect of different variants of the hinge region of the Fc fragment on the protease susceptibility of embodiments of the invention in which CCN5(dIII) is fused to the Fc fragment of IgG, wherein the tested fusion protein comprises a sequence as depicted in depicted in SEQ ID No. 28; SEQ ID No. 29, and SEQ ID No. 30, respectively, cf. example 6 below.

FIG. 3 shows the propensity of aggregation of an embodiment of the invention depending on the structure of the peptide linker connecting CCN5(dIII) with the Fc fragment of IgG, wherein the tested fusion protein comprises a sequence as depicted in depicted in SEQ ID No. 30 and SEQ ID No. 31.

FIG. 4 illustrates a fusion protein according to the present invention, comprising the TSP-1 repeat homology domain C-terminally linked to a peptide linker, and viaan Fc-hinge to an Fc-fragment.

FIG. 5 shows the reduced susceptibility to endopeptidase cleavage when the embodiment of the invention incorporates a mutation of Proline 195 of the CCN5TSP-1 repeat homology domain, as depicted in SEQ ID No 7 (Fc-HLn8-CCN5(dIII)-P195A, SEQ ID No. 41) relative to the wild-type P195 variant of the CCN5 TSP-1 repeat homology domain (Fc-HLn8-CCN5(dIII), SEQ ID No. 40).

FIG. 6 shows the production of a protein corresponding to SEQ ID NO: 58, purified by protein A capture chromatography. It can be seen that a dimer is present in the absence of the reducing agent beta-mercaptoethanol (− lane). However, in the presence of beta-mercaptoethanol (+ lane), it can be seen that the primary product is a cleavage fragment comprised of the Fc fragment only, rather than the intact fusion protein containing all of the parts encoded by SEQ ID NO: 58 (the TSP-1 homology domain fragment, the peptide linker and the Fc fragment).

FIG. 7 shows the production of a protein corresponding to SEQ ID NO: 27, having a truncated C terminal tail, purified by protein A capture chromatography. It can be seen that the protein is significantly more resistant to protease degradation that the protein corresponding to SEQ ID NO: 58, which has the C terminal tail included.

FIG. 8 shows the production of a protein corresponding to SEQ ID NO: 73, analogous to the protein corresponding to SEQ ID NO: 27, purified by protein A capture chromatography. Again, it can be seen in the presence of beta-mercaptoethanol (+ lane) that the protein is more resistant to protease degradation that the protein corresponding to SEQ ID NO: 58.

FIG. 9 shows the results of an assay measuring Phospho-AKT (Ser-473) levels in A549 lung cancer cells following administration of varying concentrations of a protein corresponding to SEQ ID NO: 41 produced in stably transfected cells. It can be seen that the protein shows no inhibition of the phosphorylation of AKT.

FIG. 10 shows the results of an assay measuring Phospho-AKT (Ser-473) levels in A549 lung cancer cells following administration of varying concentrations of a protein corresponding to SEQ ID NO: 80 produced in stably transfected cells. It can be seen that the protein shows no significant inhibition of the phosphorylation of AKT and indeed may even result in increased phospho-AKT at a higher concentration.

FIG. 11 shows the results of an assay measuring Phospho-AKT (Ser-473) levels in A549 lung cancer cells following administration of varying concentrations of a protein corresponding to SEQ ID NO: 80 produced in transiently transfected cells. It can be seen that, when produced in transiently transfected cells, the protein has a concentration dependent inhibitory activity on the phosphorylation of AKT.

FIG. 12 shows the results of an assay measuring Phospho-AKT (Ser-473) levels in A549 lung cancer cells following administration of varying concentrations of proteins corresponding to SEQ ID NOs: 84, 94 and 106. It can be seen that each of these proteins has a concentration dependent inhibitory activity on the phosphorylation of AKT.

FIG. 13 shows the results of an assay measuring Phospho-AKT (Ser-473) levels in A549 lung cancer cells following administration of varying concentrations of a protein corresponding to SEQ ID NO: 88. It can be seen that the protein is able to inhibit the phosphorylation of AKT at concentrations above 10 μg/ml.

FIG. 14 shows the results of an assay measuring Phospho-AKT (Ser-473) levels in A549 lung cancer cells following administration of varying concentrations of proteins corresponding to SEQ ID NOs: 102 and 97. It can be seen that both proteins have a concentration dependent inhibitory activity on the phosphorylation of AKT.

FIG. 15 shows the results of an assay measuring Phospho-AKT levels in A549 lung cancer cells following administration of varying concentrations of a protein corresponding to SEQ ID NO: 110. It can be seen that the protein has a concentration dependent inhibitory activity on the phosphorylation of AKT.

FIG. 16 shows the results of a number of experiments involving a protein corresponding to SEQ ID NO: 106.
A) shows that the protein inhibits the migration of human lung fibroblasts induced by both TGF-beta and CCN2.
B) shows that the protein inhibits the closure of a scratch wound induced by both TGF-beta and CCN2.
C) shows that the protein results in partial inhibition of TGF-beta induction of expression of the gene COL1A1, which is known to be pro-fibrotic.
D) shows that the protein results in partial inhibition of TGF-beta induction of expression of the gene FN1, which is known to be pro-fibrotic.
E) shows that the protein results in partial inhibition of TGF-beta induction of expression of the gene ΔCTA2, which is known to be pro-fibrotic.
F) shows that the protein results in partial inhibition of TGF-beta induction of expression of the gene CCN2, which is known to be pro-fibrotic.

### DETAILED DESCRIPTION OF THE INVENTION

The present invention is as mentioned based upon the surprising findings that the thrombospondin type 1 repeat (TSP-1) homology domain of CCN5 is a fully active structure conferring the cell signaling functions of CCN5/WISP2. Based on this new insight on the activity of the TSP-1 repeat homology domain of CCN5, the inventors provide proteins, recombinant proteins and fusion proteins according to the present invention that may be used to inhibit or counteract the cell signaling and cell physiological functions ascribed to four-domain CCN proteins, i.e. CCN1, CCN2, CCN3, CCN4 and CCN6. This new insight is considered critical for allowing the formation of a stable, homogenous, drug-like molecule based on CCN5, as it previously has not been revealed which part of the full-length CCN5 that is necessary to recapitulate the activity of CCN5, and as full-length CCN molecules are highly susceptible to proteolysis and difficult to produce in active, homogenous forms. Furthermore, this connotation that a specific part of CCN5 can be sufficient to recapitulate the activity observed upon e.g. transient overexpression of a full-length protein is also in contrast to the prevailing opinion in the field that the CCN proteins works as matricellular proteins. The prevailing opinion for the mechanism of action of the CCN proteins, and matricellular proteins in general, is that the different segments of the CCN proteins interact with various other ECM proteins and cell surface receptors thereby modulating their activity rather than working as direct modulators of cell signaling. The new knowledge of the activity of the TSP-1 repeat homology domain of CCN5, and the knowledge of the structurally close relationship with other members of the CCN protein family suggest that also the TSP-1 repeat homology domains of other CCN family proteins may also be utilized to inhibit the cell signaling functions of the four-domain CCN family proteins. According to one aspect, the recombinant proteins and fusion proteins of the present invention inhibits the phosphorylation of AKT (Ser473) in A549 cells.

Inhibition of said cell signaling is of relevance in the treatment of various disorders. CCN2 is, for example, implicated in several diseases, in particular diseases in which enhanced fibrogenesis and tissue fibrosis are a characteristic pathophysiological feature.

For example, it has been shown that overexpression of CCN2 alone is sufficient to induce fibrosis in the lung (cf. Sonnylal et al., Arthritis Rheum 62, 1523-1532 (2010)). CCN2 has also been found to be necessary for bleomycin-induced pulmonary fibrosis (Bonniaud, P. et al. Am J Respir Cell Mol Biol 31, 510-516 (2004)), radiation-induced pulmonary fibrosis (Bickelhaupt, S. et al. J Natl Cancer Inst 109 (2017), and pulmonary fibrosis due to loss of PTEN (Phosphatase And Tensin Homolog) expression (Parapuram, S. K. et al. Matrix Biol 43, 35-41 (2015)). Furthermore, in the absence of other eliciting agents, CCN2 has been found to induce pulmonary fibrosis when it is expressed and secreted from pulmonary Clara cells (Wu, S. et al. Am J Respir Cell Mol Biol 42, 552-563 (2010)), alveolar type II epithelial cells (Chen, S. et al. Am J Physiol Lung Cell Mol Physiol 300, L330-340), when expressed from a fibroblast specific promoter (Sonnylal et al (2010), supra, Sonnylal, S. et al., J Cell Sci 126, 2164-2175 (2013)) or delivered by adenovirus (Bonniaud, P. et al., Am J Respir Crit Care Med 168, 770-778 (2003)). Thus, multiple reports all support the conclusion that CCN2 is not only sufficient to elicit fibrosis in the skin or the lung, but also necessary for a full blown fibrotic phenotype in several disease models. Pulmonary fibrosis is the hallmark of the human disease idiopathic pulmonary fibrosis (IPF), however it also occurs in the setting of chronic obstructive pulmonary disease (COPD) (Jang, J. H. et al., COPD 14, 228-237 (2017)), and systemic sclerosis. In fact, pulmonary fibrosis has been reported to be the primary cause of death in up to 40% of systemic sclerosis patients (Tyndall A J et al., Ann Rheum Dis. 2010 October; 69(10):1809-15). CCN2, and other CCN proteins, such as WISP1, have also been implicated in the pathophysiology of both IPF (Konigshoff, M. et al., J Clin Invest 119, 772-787 (2009) and COPD (Jang et al, supra) in human patients.

Another example is neoplastic disorders. For example, in the setting of breast cancer CCN2 has been shown to contribute to bone metastasis in a triple negative breast cancer model (MDA-MB-231) (Kang, Y. et al., Cancer Cell 3, 537-549 (2003)). Furthermore, knockdown of CCN2 in triple negative breast cancer cells (MDA-MB-231), a cell line that expresses high levels of CCN2 (Chen, P. S. et al., J Cell Sci 120, 2053-2065 (2007)), reduced the migratory ability of these cells, while overexpression of CCN2 in the hormone receptor-positive MCF-7 breast cancer cell line, with low endogenous CCN2 expression (Chen et al, supra), increased the migratory ability of the latter cells (Chen et al, supra, Chien, W. et al., Int J Oncol 38, 1741-1747 (2011)). A later report also found that overexpression of CCN2 in MCF-7 cells increased chemoresistance, while knockdown of CCN2 in MDA-MB-231 cells decreased chemoresistance (Wang, M. Y. et al., Cancer Res 69, 3482-3491 (2009)). The increase in chemoresistance conferred by CCN2 has also been reported for other breast cancer cells (Lai, D et al., Cancer Res 71, 2728-2738 (2011)). Furthermore, through overexpression or knockdown studies it has also been shown that CCN2 contributes to epithelial-to-mesenchymal transition (EMT) and an increased ability of anchorage independent growth (mammosphere formation) of breast cancer cells (Chen et al, supra, Zhu, X. et al., Oncotarget 6, 25320-25338 (2015)). The finding of both increased chemoresistance and enhanced EMT induced by CCN2 is in line with the connection made between EMT and chemoresistance also in other cancer types (Fischer, K. R. et al., Nature 527, 472-476 (2015), Zheng, X. et al., Nature 527, 525-530 (2015)).

In a particular aspect, the present invention provides a monomeric fusion protein as defined above comprising a polypeptide corresponding to at least a portion of the thrombospondin type 1 repeat (TSP-1) homology domain of a CCN family protein, wherein the TSP-1 homology domain sequence may be truncated and/or modified, but wherein the cysteine residues of the domain are conserved. This polypeptide may for convenience be referred to herein as a “TSP-1 polypeptide”, and this term is accordingly to be understood as not conveying or implying any limitation to a specific native TSP-1 homology domain sequence only. The term “TSP-1 polypeptide” may be used synonymously or interchangeably with “TSP-1 domain protein” or TSP-1 domain sequence”.

As demonstrated in the examples below, it has surprisingly been found that monomeric fusion partners are advantageous in producing active and stable proteins as compared to dimeric fusion partners, such as Fc fragments derived from IgG proteins, which produce dimeric fusion proteins. Monomeric fusions retain the activity of the TSP-1 domain polypeptide that they comprise. Furthermore, the proteins are stable, including with respect to proteolytic degradation. As described further below, resistance to proteolytic degradation may be improved by making modifications to the amino acid sequence of the TSP-1 polypeptide, including in particular the Ala substitution referred to above.

Accordingly, the polypeptide of component (i) of the fusion protein may comprise insertions, deletions, substitutions, mutations or any combination thereof, relative to said sequence selected from SEQ ID NOs: 37 or 2 to 6, or to the sequence selected from SEQ ID NOs: 1 or 8 to 12, provided that the polypeptide retains at least 80% sequence identity with said sequence and that all of the cysteine residues in said sequence are conserved.

In another aspect, the invention provides a protein (e.g. a recombinant protein) which consists of or comprises a polypeptide corresponding to at least a portion of the thrombospondin type 1 repeat (TSP-1) homology domain of a CCN family protein but not in the context of a fusion protein, wherein the TSP-1 domain sequence may be truncated and/or modified and comprises an Ala substitution at the position corresponding to position 2 of SEQ ID NOs: 37 or 2 to 6, or SEQ ID NOs: 1 or 8 to 12, but wherein the cysteine residues of the domain are conserved. In other words the TSP-1-domain protein may be provided without, or independently of, another component such as a fusion partner. Thus, the TSP-1 domain protein is not fused to or linked to another protein domain or component or other functional or structural protein sequence. For convenience such proteins may be referred to as “Ala-substituted proteins”.

As used herein, the term “conserved” means that a residue in a given sequence is not deleted or substituted. In other words the term “conserved” is being used synonymously (and interchangeably) with the term “retained”. It simply means that the cysteine residues are not removed from the sequence. Accordingly, in the context above it means that the cysteine residues in the sequence selected from SEQ ID NOs: 37 or 2 to 6 or 1 or 8 to 12 are not deleted or substituted. It is noted that the insertion of additional residues between conserved residues (e.g. between conserved cysteines), or the deletion of non-conserved (e.g. non-cysteine) residues, may alter the position of the conserved residues in the polypeptide sequence relative to their position in the original reference sequence (e.g. the sequence selected from SEQ ID NOs: 37 or 2 to 6). However, such residues are still considered to be “conserved”, as defined herein. Thus, the term “conserved” does not imply any restriction or limitation on the position (or more particularly, position number) of the cysteine residues.

In some embodiments, the polypeptide of (i) comprises or consists of:
(a) an amino acid sequence selected from SEQ ID NOs: 1 or 8 to 12; or
(b) an amino acid sequence having at least 80% sequence identity to a sequence selected from SEQ ID NOs: 1 or 8 to 12; or
(c) a part of an amino acid sequence of (a) or (b), wherein said part comprises at least the 44 amino acid sequence of SEQ ID NOs: 37, 6, 2, 3, 4 or 5 respectively or a sequence having at least 80% sequence identity to a sequence selected from SEQ ID NOs: 37, 6, 2, 3, 4 or 5 respectively.

As noted above, the monomeric fusion partner of (ii) and the peptide linker of (iii) are not or do not comprise an IGF binding protein homology domain, a von Willebrand factor type C repeat homology domain, or a cysteine knot domain of a CCN family protein. Alternatively put, the only domain of a CCN family protein that may be present in the fusion protein of the present invention is a TSP-1 homology domain.

Similarly and analogously, in the context of the Ala-substituted proteins which are not fusion proteins, the protein does not comprise any other CCN domain (beyond the TSP-1 domain protein).

In some embodiments, the polypeptide of (i), or the Ala-substituted protein, may comprise only part of a TSP-1 homology domain, as defined above. The present inventors have found that the minimum fragment of the TSP-1 domain required is the 44 amino acid sequence of SEQ ID NOs: 37, 6, 2, 3, 4 or 5. Accordingly, in some embodiments, the polypeptide of (i) is at least 44 amino acids in length. In some embodiments, the polypeptide of (i) is 44 to 57 amino acids in length. However, as noted above, there may be one or more deletions of amino acids in the 44 amino acid minimum fragment which lie between the cysteine residues. Thus, in some embodiments the length of the TSP-1 polypeptide may be less than 44 residues, i.e. 40-43 residues.

In some embodiments, the polypeptide of (i) consists of an amino acid sequence selected from SEQ ID NOs: 37 or 2 to 6, or a sequence having at least 80% sequence identity to a sequence selected from SEQ ID NOs: 37 or 2 to 6.

As described above, the proteins of the invention, including fusion proteins, exhibit (or in other words, demonstrate or have) the activity, more particularly the biological activity, of CCN5. In an embodiment, the proteins may retain or exhibit or have the activity of the TSP-1 homology domain of CCN5. Alternatively, the proteins may be defined as exhibiting (or demonstrating or having) the activity, particularly biological activity, of an isolated TSP-1 homology domain of a CCN protein. The foregoing may apply to any activity of the domain, and particular activities which reflect the anti-fibrotic effect of the TSP-1 homology domain. Such an activity may be assayed for (or tested or detected) using any convenient assay or method, based on any particular biological effect of the domain.

It is noted that the activity of a given protein may conveniently be assessed by analyzing the effect of the protein on the phosphorylation of AKT. In particular, a given protein may be assayed for its ability to inhibit the phosphorylation of AKT (Ser-473) in A549 human lung cancer cells, as described in Example 2. The skilled person will appreciate that other similar assays may be devised to assess the same activity, or to assess other related anti-fibrotic activities.

As noted above, in other aspects of the present invention, recombinant proteins that inhibit or counteract the cell signaling and cell physiological functions ascribed to four-domain CCN proteins are provided, comprising an amino acid sequence according to formula I above.

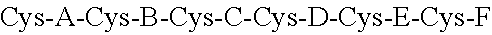

wherein A, B, C, D, E and F is as defined above and in the appended claims. Formula I is a result of the alignment of TSP-1 repeat homology domain of the structurally related CCN family proteins (CCN 1-CCN6), all comprising 6 conserved cysteins, and taking account that amino acids may be substituted without affecting the activity of the protein (conservative substitutions as discussed further below). The position of the first conserved cysteine of the TSP1 repeat homology domain of the different CCN proteins is defined as position #1 of the recombinant protein of formula I.

The five segments between the conserved cysteines are A, B, C, D, and E, respectively.

The first segment A is defined by the formula A1-A2-A3-A5-A6-A7-A8-A9, wherein A1-A9 is as defined above. The amino acid in position #7 (A7) of segment A is tryptophan (W) in all the members of the CCN family proteins and is believed to be conserved.

The second segment B is defined by the formula B1-B2-B3, wherein B1-B3 is as defined above. According to one embodiment, B1 and B3 is either serine or Threonine.

The third segment C is defined by the formula C1-C2-C3-C4-C5-C6-C7-C8-C9-C10-C11-C12-C13-C14, wherein the amino acids C1-C14 is as defined above. According to one embodiment, the amino acids C1 and C3 is glycine (G). According to another embodiment, C7 is Arginine (R), C10 and C12 is both asparagine (N).

The fourth segment D is defined by the formula D1-D2-D3-D4-D5-D6-D7-D8, wherein the amino acids D1-D8 is as defined above. According to one embodiment, D7 is Arginine (R).

The fifth segment E is defined by the formula E1-E2-E3-E4, wherein the amino acids E1-E4 is as defined above. According to one embodiment of the invention, E4 is proline.

Following the last cysteine is a carboxyl-terminal peptide segment of variable length (F) comprising from 0 to 13 amino acids.

F may be deleted or shortened compared with the amino acid sequences of the TSP-1 repeat homology domain of the CCN family proteins. According to one embodiment, F is absent. According to another embodiment, F consists of a peptide selected from the group consisting of PPSRGRSPQNSAF, GQPVYSSL, EADLEEN, EQEPEQPTD, DVDIHTLI, and DSNILKTIKIP. According to one aspect of this embodiment, the recombinant proteins may take the form of an amino acid sequence as depicted in SEQ ID No. 8-12.

According to another aspect, the present invention provides recombinant proteins comprising an amino acid sequence selected from the group consisting of SEQ ID No. 1, SEQ ID No. 2, SEQ ID No. 3, SEQ ID No. 4, SEQ ID No. 5, SEQ ID No. 6, and SEQ ID No. 7, SEQ ID No.8, SEQ ID No. 9, SEQ ID No. 10, SEQ ID No. 11, SEQ ID No. 12, SEQ ID No. 37, SEQ ID No. 38; and fragments or variants thereof having at least 50% sequence identity with the amino acid sequences SEQ ID No. 1, SEQ ID No. 2, SEQ ID No. 3, SEQ ID No. 4, SEQ ID No. 5, SEQ ID No. 6, and SEQ ID No. 7, SEQ ID No.8, SEQ ID No. 9, SEQ ID No. 10, SEQ ID No. 11, SEQ ID No. 12, SEQ ID No. 37, SEQ ID No. 38.

According to one aspect, a recombinant protein is provided, consisting of an amino acid sequence selected from the group consisting of SEQ ID No. 1, SEQ ID No. 2, SEQ ID No. 3, SEQ ID No. 4, SEQ ID No. 5, SEQ ID No. 6, and SEQ ID No. 7, SEQ ID No.8, SEQ ID No. 9, SEQ ID No. 10, SEQ ID No. 11, SEQ ID No. 12, SEQ ID No. 37, SEQ ID No. 38; and fragments or variants thereof having more than 50% sequence identity with the amino acid sequences SEQ ID No. 1, SEQ ID No. 2, SEQ ID No. 3, SEQ ID No. 4, SEQ ID No. 5, SEQ ID No. 6, and SEQ ID No. 7, SEQ ID No.8, SEQ ID No. 9, SEQ ID No. 10, SEQ ID No. 11, SEQ ID No. 12, SEQ ID No.37, SEQ ID No. 38.

“Recombinant proteins” as used herein are proteins encoded by recombinant nucleic acids. They are expressed from recombinant nucleic acids in a host cell as further disclosed below.

“Recombinant nucleic acid” is used herein to describe a nucleic acid molecule which, by virtue of its origin or manipulation is not associated with all or a portion of the polynucleotide with which it is associated in nature and/or is linked to a polynucleotide other than that to which it is linked in nature as further disclosed below.

The skilled person will acknowledge that modification of the amino acid sequence of the recombinant proteins and fusion proteins according to the present invention may be introduced without altering the activity of said protein. Amino acids are commonly classified as hydrophobic or hydrophilic and/or as having polar or non-polar side chain. Substitutions of one amino acid for another having the same biochemical characteristics are commonly known as conservative substitution.

Conservative substitutions of amino acids include substitutions made among amino acids within the following groups:
MILV
FYW
KRH
AG
ST
QN
ED

Generally, a conservative amino acid substitution refers to an amino acid substitution that does not alter the relative charge or size characteristics of the protein in which the amino acid substitution is made, and thus seldom alter the structure of the protein, which is why the biological activity are neither altered significantly.

The skilled person will acknowledge that the biological activity of a protein also may be retained if one or a few amino acids are deleted, inserted or added to an amino acid sequence, as long as structural and chemico-physical properties are conserved.

The symbol “Δ” when used herein in front of an amino acid refers to the deletion of the indicated amino acid, e.g. ΔK447 is to be understood as a protein in which K447 is not present. A deletion of a specific amino acid is also herein alternatively indicated with a “−”, e.g. K447− is also to be understood as a protein in which K447 is not present.

It is thus to be understood that the present invention encompasses recombinant proteins and fusion proteins as disclosed in the appended claims, wherein such modifications as described above (substitutions, deletions, insertions and additions of amino acids) may be introduced without essentially altering their biological activity, i.e. ability to inhibit or counteract the cell signaling and cell physiological functions ascribed to the four-domain CCN-family proteins; CCN1, CCN2, CCN3, CCN4 and CCN6.

Throughout the present specification, references are made to amino acid sequences. When referring to amino acid sequences herein, reference is sometimes made to modification of the amino acid sequence or protein in question by reference to “uniprot numbering” or Eu numbering in the present specification. Uniprot numbering refers to the numbering utilized in the uniprot database (UniProt Consortium, Nucleic Acids Res. 2019 Jan. 8; 47(D1):D506-D515). Uniprot numbering is used when referring to amino acid numeration of the CCN proteins. Eu numbering refers to the numbering of the Eu antibody (Edelman et al., 1969, Proc Natl Acad Sci USA 63:78-85), and is used when referring to amino acids in the Fc-fragments of human IgG subclasses with or without mutations or chimeras different from the wild-type. The Eu numbering system is accessible e.g. from the international ImMunoGeneTics information system (IMGT) in the IMGT Scientific chart. The IMGT is described in Lefranc M-P, Biomolecules. 2014 December; 4(4): 1102-1139.

As used herein, when referring to “sequence identity”, a sequence having at least x % identity to a second sequence means that x % represents the number of amino acids in the first sequence which are identical to their matched amino acids of the second sequence when both sequences are optimally aligned via a global alignment, relative to the total length of the second amino acid sequence. Both sequences are optimally aligned when x is maximum. The alignment and the determination of the percentage of identity may be carried out manually or automatically.

Alignment for purposes of determining percent amino acid sequence identity can be achieved in various ways that are within the skill in the art, for instance, using publicly available computer software such as ClustalOmega (Sievers F, Higgins DG (2018) Protein Sci 27:135-145), Protein BLAST (from National Center for Biotechnology Information (NCBI), USA) or commercially available software such as Megalign (DNASTAR) software. Those skilled in the art can determine appropriate parameters for measuring alignment, including any algorithms needed to achieve maximal alignment over the full length of the sequences being compared. NCBI BLAST is another example of software used to determine amino acid sequence identity (MacWilliam et al., Nucleic Acids Res. 2013 July; 41(Web Server issue): W597-W600).

According to one aspect of the invention, a recombinant protein is provided comprising an amino acid sequence selected from the group consisting of SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 37 and SEQ ID No. 38; and fragments or variants thereof having at least 50% sequence identity with the amino acid sequences SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 37 and SEQ ID No. 38.

According to another aspect, a recombinant protein is provided comprising an amino acid sequence having at least 60%, 70%, 80%, 90%, or 95% sequence identity with an amino acid sequence selected from the group consisting of SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 37, and SEQ ID No. 38.

Biologically active proteins and peptides have an important place in clinical management of human disease. However, many proteins and peptides present challenges due to having less than ideal pharmacokinetic properties, either because they are eliminated by kidney filtration due to their small size and/or due to proteolytic metabolism. Such factors may impose limitations or challenges upon administration of the drug to a subject in need of treatment such as having to administer constant infusions or frequent subcutaneous administrations to keep the circulating concentrations of protein or peptide at an effective therapeutic level. The need for constant or very frequent administration of a drug is clinically undesirable due to the obvious challenges and inconvenience for both patient and physician.

One strategy for extending half-life of a biologically active peptide or protein is to link a polyethylene glycol (PEG) group to the peptide or protein of interest, by a process called PEGylation (cf. e.g. Dozie et al. (2015), Int. J. Mol Sci, 16(10) 25831-25864). The general strategy for protein PEGylation is to react a functional group on a protein with a complementary group on a PEG molecule to form a protein-PEG conjugate. The PEG moiety offers several advantages for increasing a protein's stability and circulating half-life, due to its flexibility, hydrophilicity, variable size and low toxicity.

In one embodiment the present invention thus provides a recombinant protein as described above, wherein the said protein is pegylated. The fusion proteins according to the present invention may also be pegylated.

### Fusion Proteins

Another way of obviating the challenges related to medical use of peptides and proteins is obtaining an extension in half-life for the bio-active protein or peptide by making fusion proteins (cf. e.g. Valeria et al. (2017), “A New Approach to Drug Therapy: Fc-Fusion Technology), Prim Health Care, 7:255, doi:10.4172/2167-1079.1000255). By covalently fusing the protein or peptide to a carrier protein via genetic recombination one can increase the molecular weight of the protein of interest to approximately 60 to 70 kDa, which is the threshold for renal filtration.

The present invention provides a fusion protein comprising
(i) Thrombospondin type 1 repeat (TSP-1) homology domain of a CCN family protein;
(ii) fusion partner N- or C-terminally fused to the TSP-1 repeat homology domain of (i) and wherein said fusion partner is selected from the group consisting of serum albumin, transferrin and immunoglobulin Fc-fragment.
(iii) optionally a peptide linker between the TSP-1 repeat homology domain and the Fc fragment (being N- or C-terminally fused to the TSP-1 repeat homology domain) of (i).

Throughout this specification, the TSP-1 repeat homology domain may also be denoted and refer to domain III, referring to the third domain of the CCN family proteins.

In one preferred aspect, the fusion partner is a monomeric fusion partner, and results in a fusion protein which is monomeric. Such fusion proteins, and in particular the TSP-1 domains thereof, are defined above and described further below.

However, the present disclosure also includes other embodiments, both with respect to the TSP-1 domain protein component and the fusion partner component.

According to one such embodiment, the TSP-1 repeat homology domain is a recombinant protein of formula I as defined above.

The TSP-1-repeat-homology-domain is according to another embodiment a recombinant protein having an amino acid sequence as defined in any one of the sequences depicted in SEQ ID No. 1-12, 37, and 38, or a recombinant protein of formula I as defined above.

According to one embodiment, the TSP-1 repeat homology domain is a recombinant protein comprising an amino acid sequence having at least 50%, 60%, 70%, 80%, 90%, or 95% sequence identity with an amino acid sequence selected from the group consisting of SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 37, and SEQ ID No. 38.

Proteins are inherently susceptible to protease degradation. In order to prevent protease degradation of the recombinant proteins and fusion proteins according to the present invention, modifications to the amino acid sequence may be introduced, e.g. by site directed mutagenesis, in order to provide protease resistant recombinant proteins and fusion proteins. For example, a point mutation may be introduced in the thrombospondin type 1 repeat (TSP-1) homology domain of a CCN family protein as defined in SEQ ID No. 1-12, 37 or 38, or more particularly a protein as defined in any one of SEQ ID NO.s 1-6, 8-12 or 37, of the fusion protein or in the recombinant protein. According to one embodiment, a point mutation is introduced decreasing the susceptibility of proteolytic degradation. A non-limiting example of a point mutation resulting in less proteolysis of the recombinant proteins and fusion proteins of the present invention is by introducing a point mutation corresponding to the replacement of proline with alanine in position 195 (P195A) of domain III of CCN5, such as shown in SEQ ID No. 7. A similar mutation may also be introduced in the amino acid sequences originating from domain III of other CCN family members. SEQ ID NO. 38 represents the truncated 44 amino acid sequence of the TSP-1 domain of CCN5 comprising the Ala substitution. SEQ ID NOs. 42-46 represent the truncated 44 amino acid sequences of the TSP-1 homology domains of CCN1, 2, 3, 4 and 6 respectively comprising the Ala substitution. SEQ ID NOs. 47 to 51 represent the longer TSP-1 homology sequences of CCN1, 2, 3, 4 and 6 respectively comprising the Ala substitution. Any such sequence, or a sequence having at least 80% sequence identity therewith, may be used according to the present invention.

As noted above, in a preferred embodiment, the fusion partner (ii) of the fusion protein according to the present invention is monomeric. Any monomeric fusion partner may be used. Thus the fusion partner may be any protein, or part thereof (e.g. protein domain), which occurs and remains in monomeric form when fused to the TSP-1 homology domain protein component. Thus the fusion protein comprising the monomeric fusion partner and the TSP-1 homology domain protein remains as a monomer. That is, it does not dimerise or form higher multimers with itself.

Various proteins are known as possible fusion partners and may include native proteins, or fragments or amino acid sequence-modified variants thereof, as well as synthetic proteins or amino acid homopolymers. Such proteins include notably Fc fragments of IgG, serum albumin or transferrin.

A fusion partner is defined broadly herein as a second polypeptide (or second amino acid sequence) which is not present in combination with (e.g. adjacent to, or linked to, directly or indirectly) the first CCN TSP-1 homology polypeptide in nature, and which is linked to the first CCN TSP-1 homology polypeptide in a synthetic or artificial combination. Thus, a fusion protein comprises a non-native combination of at least two amino acid sequences or polypeptides linked, or fused together.

The fusion partner may be an amino acid sequence which is at least 6, 8, 9, 10, 15, 20, 25, 30, 40 or 50 or more amino acids long. Typically, the fusion partner is a functional polypeptide, or in other words it is a polypeptide which imparts a function or property to the fusion protein, e.g. to stabilise the fusion protein (to make the first polypeptide more stable), or to increase its serum half-life. Thus the fusion partner may be a structural protein or have a structural function, or it may impart an activity or property to the fusion protein, e.g. a binding activity (e.g. the fusion partner may be a member of a binding pair, or it may be an affinity binding partner etc.). In representative examples the fusion partner may be an albumin (in particular a serum albumin), a fibrinogen, a glutathione S-transferase, a transferrin, streptavidin or a streptavidin-like protein, or an immunoglobulin, or a part thereof, in particular the Fc portion of an immunoglobulin (e.g. IgG1, IgG2, IgG3 or IgG4), or a part or modification thereof. Suitable serum albumins include bovine serum albumin (BSA), mouse serum albumin (MSA) and in particular human serum albumin (HSA). Other possible fusion partners include polypeptides which may act to improve pharmacokinetic properties of the fusion protein, e.g. synthetic polypeptides, such as a homo amino acid polymer, a proline-alanine-serine polymer, or an elastin-like peptide, for example as described in Strohl, 2015, BioDrugs 29, 215-239. Any fusion partner known in the art for use with therapeutic proteins may be used.

In an embodiment, the fusion partner (ii) of a fusion protein according to the present invention may be either an Fc-fragment of IgG (any subclass or chimera of any subclasses), serum albumin or transferrin.

The fusion partner may be coupled N- or C-terminally to the TSP-1 homology domain protein component of the fusion protein, e.g. to the TSP-1 repeat homology domain of CCN5 or any of the other CCN proteins, as defined herein. It may be linked directly, or indirectly, via a linker, as described further below.

Fc fragments tend to form dimers, and when used in fusion proteins, the fusion protein construct tends to comprise two copies of the fusion protein. However, it is known in the art that monomeric Fc fragments and monomeric fusion proteins comprising them may be prepared.

Accordingly, where the fusion partner is an Fc-fragment, it is preferably a monomeric Fc-fragment, such as a monomeric Fc-fragment of human IgG, of any class. Chimeric Fc fragments comprising parts of Fc regions from different classes are encompassed, as are Fc fragments with modified sequences.

Fc-fusion proteins are a growing class of protein therapeutics based on chimeric proteins consisting of an effector domain coupled to the Fc-fragment of an IgG-isotype. A typical example of a biopharmaceutic product is etanercept (TNF-α receptor coupled to an Fc fragment) used in treatment of e.g. rheumatoid arthritis. Another example of an Fc fusion biopharmaceutical protein is aflibercept. Aflibercept, a VEGF receptor-Fc-fusion protein used in the treatment of wet macular degeneration and metastatic colorectal cancer. The principal rationale for making Fc fragment fusion proteins is to obtain extension of half-life due to increase of molecular mass sufficient to exclude renal excretion and to enhance renal proximal tubular reabsorption via the neonatal Fc-receptor. Also, pH-dependent binding of Fc-fusion proteins to the neonatal Fc receptor (FcRn) on endothelial cells allow Fc-based fusion proteins that would otherwise be destined for endocytosis and subsequent lysosomal degradation to be recycled and released back into circulation.

According to one embodiment, a fusion protein is provided wherein the fusion partner (ii) is an Fc-fragment from a human IgG (immunoglobulin G, also known as immunoglobulin γ), including all subclasses of human IgG. According to yet another embodiment of the present invention, a fusion protein is provided, wherein the fusion partner is the Fc fragment of IgG1, IgG2 or IgG4. Preferably, the Fc-fragment of human IgG is that of subclass IgG4 (SEQ ID. No 13) or IgG2 (SEQ ID. No 14).

IgG1, IgG2 and IgG4 are often preferred to IgG3 due to their longer half-lives of approximately 3 weeks. The skilled person will acknowledge that the choice of the IgG isotype of a particular subclass as an Fc fusion partner will depend on the desired half-life extension and cytotoxic level of activity for the final compound. Therapeutic antibodies that are indicated for the treatment of cancer or autoimmune diseases belong, for the most part, to the IgG1 subclass because of their high affinity to Fc receptors and potent ability to exert immune-effector functions. IgG2 and IgG4 are, on the other hand, preferred subclasses of IgG for use as the backbone of a therapeutic candidate when a lack of immune-effector functions is desired, as the immune-effector functions may cause adverse effects. The propensity of the Fc-fragment to activate immune effector functions is dependent upon the Ig isotype and subclass and varies for different immune effector functions. In addition to selecting an Fc-fragment of a suitable IgG subclass, the amino acid sequence of the IgG subclass may be modified, e.g. by site directed mutagenesis, in order to reduce the Fc-fragments capacity to activate immune-effector functions.

Fc fragments may be selected which form monomers, or more precisely which retain or have monomeric form, or may be modified to introduce mutations which allow, or facilitate a monomeric structure. Such mutations are termed herein “monomer-generating mutations” Examples of Fc fragments which comprise monomer generating mutations are SEQ ID NOs. 54 and 55. The person skilled in the art knows how to introduce such mutations and select monomeric Fc mutants.

### Avoidance of Immune Effector Activation Functions of Fc Fragmen's

For instance, in the biopharmaceutic fusion protein dulaglutide (Trulicity™), a GLP-1 agonist -Fc-fragment fusion protein used in the once weekly treatment of type 2 diabetes, the well characterized mutations F234A and L235A are introduced in the hinge region of an IgG4 Fc-fragment to reduce the capacity of activating immune effector functions.

According to one embodiment of the invention, the Fc fusion partner is an Fc-fragment of IgG4, wherein the IgG4 Fc fragment is modified in order to avoid immune effector functions, e.g. comprising the above F234A and L235A mutations.

### Protease Resistant Fc Fragments

Another factor that can reduce both yields in manufacturing and the biological half-life is endopeptidase cleavage of the fusion protein. To reduce or eliminate the risk of proteolytic degradation, modifications may be introduced in the amino acid sequence of the Fc fragment, in particularly by introducing mutation in the sites susceptible of proteolytic cleavage. In EP patent application EP2654780B1, the Fc domain of IgG1 constant region was modified by replacing E233-L234-L235-G236 with P233-V234-A235 (deleting G236) (EU numbering) to render the resulting modified Fc-containing protein resistant towards proteolytic degradation.

Incorporation of the amino acid modification disclosed in EP2654780B1 in an Fc fragment of IgG4 coupled to domain III of CCN5 were considered not to provide sufficient resistance to endopeptidases. Improved protease resistance was however achieved by further modifications of the IgG subtype used as a fusion partner according to the present invention.

More specifically, it has been found that fusion proteins comprising the entire hinge region of IgG2 and the constant heavy chains 2 and 3 of IgG4 displayed superior proteolytic resistance.

In Mueller J P et al., Mol. Immunol. (1997), 34(6), pp. 441-452, the use of IgG2/IG4 chimeras in IgG antibodies are disclosed. Another biopharmaceutical monoclonal antibody named eculizumab, which are used in the treatment of paroxysmal nocturnal hemoglobinuria and atypical hemolytic uremic syndrome, has been shown to be useful e.g. in avoiding the capacity of IgG4 to activate the FcγR dependent immune effector. Furthermore, the IgG4 constant domains 2 and 3 of such a chimeric Fc-fragment are also shown to avoid the capacity of IgG2 to activate complement dependent immune effector functions. Reference is in this respect made to the report of Rother et al., (2007), cf. Nat. Biotechnol., 25(11), pp. 1256-1264 and Mueller J P et al., supra.

Also, Borrok et al. (2017), J. Pharm. Sci. 106; 1008-1017, disclose the introduction of modifications in an Fc-fragment to study its effect on immune effector functions of antibodies (FQQ-YTE mutations). In WO2017158426A1, modifications of antibodies by introducing mutations in the Fc fragment to improve the half-life of antibodies are disclosed. In particular, modifications in one or more of the positions 311, 434, 428, 438, and 435 in the Fc-region of an immunoglobulin are disclosed.

Furthermore, Kinder et al. J Biol Chem. 2013 Oct. 25; 288(43):30843-54 reports of mutations in the lower hinge of IgG1 (i.e. E233P, L234V, L235A, G236-, Eu numbering) resulted in protease resistant IgG1 antibodies.

According to one embodiment, the Fc-fragment of the fusion protein according to the present invention consists of an Fc fragment of the IgG4 subclass incorporating the following mutations; S228P, F234A, L235A, K447-, Eu-numbering, cf. SEQ ID No. 15.

Jacobsen et al. J Biol Chem. 2017 Feb. 3; 292(5):1865-1875 reported that mutation of Asn297 resulting in that the Fc fragment was aglycosylated, which further resulted in lack of IgG effector functions. Jacobsen also found that some variants (N297G) resulted in antibodies having better stability and developability compared with other variants (N297Q or N297A). Further modifications were also introduced (disulfide bridges) that resulted in better stability than the parental IgG1.

According to the present invention, when the fusion partner is an Fc-fragment, it may be aglycosylated without, or with a stabilizing disulfide bridge, such as e.g. in SEQ ID No. 16.

To the knowledge of the inventors, an Fc-fragment composed of the entire hinge region of IgG2 and constant heavy chains 2 and 3 of IgG4 has not previously been used to prepare fusions proteins by linking said Fc-fragment to an effector protein.

According to one embodiment, the fusion partner of the present fusion protein is a Fc fragment of IgG1 being aglycosylated and stabilized by a disulfide bridge, and wherein the lower hinge with the following mutations have been introduced: E233P, L234V, L235A, G236- (Eu numbering) (SEQ ID NO. 17).

According to one embodiment, the fusion partner of the fusion protein comprising the TSP-1 repeat homology domain of a CCN family protein is an Fc fragment of IgG4, and wherein the following mutations have been introduced to the lower hinge: E233P, L234V, L235A, G236- (Eu numbering) in addition to the S228P and K477-mutations (SEQ ID NO. 18).

In one preferred embodiment the Fc-fragment is a chimera of the hinge region of IgG2 (216 ERKCCVECPPCPAPPVA-GP 238, Eu numbering) and any of the other IgG subclasses. Most preferably the Fc-fragment is a chimera of the hinge region of IgG2 and the constant heavy domains 2 and 3 of IgG4 with a deletion of the carboxyl-terminal K477 (Eu numbering), as shown in SEQ ID. No. 19. This embodiment of the invention has been shown to have improved protease resistance characteristics (cf. example 6).

In one embodiment, the fusion partner of the monomeric fusion protein of the invention is an Fc-fragment of IgG1 being disulfide bridge stabilized (R292C, V302C), aglycosylated (N297G) and with monomer-generating mutations (C220Q, C226Q, C229Q, T366R, L368H, P395K, K409T, M428L), Eu numbering), as provided in SEQ ID NO: 54.

In a further embodiment, the fusion partner of the monomeric fusion protein of the invention is an Fc-fragment being a chimera of the hinge region of IgG2 and the constant heavy domains 2 and 3 of IgG4 with a deletion of the carboxyl-terminal K477- and with monomer-generating mutations (C219Q, C220Q, C226Q, C229Q, L351F, T366R, P395K, F405R, Y407E) and half-life extending mutations (M252Y, S254T, T256E) (Eu numbering), as provided in SEQ ID NO: 55.

Although a fusion protein according to the present invention is exemplified by using an Fc-fragment consisting of the entire hinge region of IgG2, the constant heavy chains 2 and 3 of IgG4, and domain III of a CCN protein family member, it is believed that the advantageous protease resistance is also achieved if such an Fc-fragment chimera is coupled to other effector molecules, e.g. such as VEGFR, FGF-21 or GLP1. The effector molecule is the part of the Fc fusion protein that affords the desired pharmacodynamics properties, while the Fc fragment contributes to the pharmacokinetic properties.

### Serum Albumin as Fusion Partner

An alternative strategy for extending the half-life of peptides and proteins is using serum albumin (SA) as a fusion partner. IgG and SA share a prolonged half-life of about 19 days compared to a few days or less for most other circulating proteins. SA also has affinity for the neonatal Fc receptor (FcRn) and is rescued from intracellular degradation (cf. Andersen et al. (2014), J Biol Chem, 289(19); pp 13492-13502).

In one embodiment of the present invention, a fusion protein is provided as described above, wherein the fusion partner is serum albumin, preferably human serum albumin.

In one embodiment, a monomeric fusion protein is provided as described above, wherein the fusion protein comprises amino acids 25 to 606 of human serum albumin, as provided in SEQ ID NO: 101.

In a further embodiment of the invention the albumin, e.g. human serum albumin, is modified, for example in order to increase or decrease half-life by altering its FcRn affinity, with or without pH-dependence resulting in increased or decreased half-life.

### Transferrin as Fusion Partner

Yet an alternative strategy for extending the half-life of peptides and proteins is using transferrin as a fusion partner, utilizing the naturally long half-life of transferrin. (Strohl W. BioDrugs. 2015; 29(4): 215-239). Transferrin may be used in its glycosylated or non-glycosylated form.

In one embodiment of the present invention, a fusion protein is provided as described above, wherein the fusion partner is transferrin, preferably human transferrin.

In one embodiment, a monomeric fusion protein is provided as described above, wherein the fusion protein comprises amino acids 20 to 698 of human transferrin, as provided in SEQ ID NO: 53.

### Linker

According to another embodiment, the fusion proteins according to the present invention may optionally comprise a peptide linker between the fusion partner and the effector molecule, i.e. the linker is being N- or C-terminally fused to the TSP-1 repeat homology domain of the CCN protein (the TSP-1 domain protein/polypeptide).

Any peptide linker may be used (as long as it is not a CCN protein sequence), many of which are known and described in the art. The linker may be a flexible linker sequence (which may include repeats of a flexible linker sequence motif). Typical linkers known in the art are rich in small non-polar (e.g. glycine) or polar (e.g. serine or threonine) residues, and commonly consist of stretches of glycine and serine residues (GS) or other amino acid residues such alanine, lysine and/or glutamate (A, K, and/or E), or indeed any amino acids. A commonly used linker is the (GGGGS) linker (SEQ ID NO: 121), which may be provided as a repeating unit in a linker (as (GGGGS)n, where the copy number of n may be adjusted, e.g. from 1-10, 1-6, 1-4 etc.). The linker may be 1-50, 1-45, 1-40, 1-30, 1-25, 1-20, 1-15, 1-12, 1-10, e.g. 1-8, 1-6, 1-5, or 1-4, amino acids long. Various different linkers are described and used in the Examples below, and any of these may be used in any of the fusion proteins of the invention.

In some embodiments, the linker comprises no more than 50 amino acids.

The properties of the peptide linker may further improve the maintenance of the effector functions. However, peptide linkers may be susceptible to endopeptidase cleavage and elimination of the fusion protein. Peptide linkers with glycine with or without serine residues interspersed are commonly utilized, however this design does not always yield fusion proteins with desired activities and resistance to endopeptidases. In US20180273603, disclosing a neurotrophin binding protein-Fc-fusion protein, the use of α-helical linkers comprising repetitions of the sequence A(EAAAK)A (SEQ ID No. 14 therein) are suggested. Furthermore, US2018/0127478 discloses the use of an amino acid linker consisting of one to three repetitions of the sequence EAAAK is suggested in an Fc-fusion protein.

According to the present invention, a linker consisting of the peptide sequence EAAAK (SEQ ID No. 21 herein) may also be incorporated between the TSP-1 homology domain and the fusion partner (Fc fragment). More preferably, the linker is composed of a repetition of the amino acid sequence EAAAK.

If a linker is included in the fusion protein of the present invention, the linker is placed between the fusion partner and the effector molecule, i.e. the domain III of the CCN protein. The linker may be introduced either C-terminally or N-terminally of domain III of the CCN protein.

Furthermore, the helical linker was resistant to endopeptidase cleavage following expression of the recombinant protein in suspension CHO cells. This is important both for manufacturing purposes and for in vivo efficacy. Furthermore, the incorporation of an α-helical linker between the Fc-fragment and the effector domain in an Fc fusion protein is shown to reduce the aggregation tendency of the Fc fusion protein.

Although these findings are shown with a fusion protein comprising domain III of CCN family protein as an effector protein, it is believed that the advantageous reduced tendency to aggregation and the protease resistance effects are also obtained if combining other effector molecules to a Fc fragment an α-helical linker according to the present invention.

The present invention therefore provides an Fc fusion protein comprising an Fc-fragment that has a peptide linker sequence of the formula aa1-aa2-(EAAAK)n-aa3-aa4-aa5, wherein n≥4, between the Fc-fragment and an effector molecule, and wherein aa1, aa2, aa3, aa4, aa5 is independently absent or an amino acid. The linker may be placed N-terminally or C-terminally of the Fc-fragment. According to one embodiment, n is 8. According to another embodiment, aa1 is Threonine (T), aa1, aa2, aa3, aa4 and aa5 is Ala (A). According to one embodiment, the linker of the above Fc-fusion protein is selected from the group consisting of SEQ ID NO. 22, SEQ ID NO. 23, SEQ ID No. 24 and SEQ ID No. 25. In particular, it has been shown that the use of a fusion protein according to the present invention comprising a linker consisting of a (EAAAK)-repeat, i.e. such as (EAAAK)n wherein n is 8 advantageously results in less aggregation.

An alternative linker that may be used according to the present invention is a linker with an amino acid sequence as depicted in SEQ ID No. 20 (TEGRMD).

In one embodiment, the invention may thus include the incorporation of a linker peptide between the fusion partner, and domain III on CCN5 (e.g. SEQ ID. No 1-SEQ ID. No. 12, 37 or 38). Non-limiting examples of fusion proteins incorporating a linker of SEQ ID No. 20 is shown in SEQ ID No. 28, SEQ ID No. 29 and SEQ ID No. 30, respectively.

In the case when the invention takes the embodiment of domain III of CCN5 genetically fused N-terminal of a peptide linker (as in SEQ ID. No 20) and an Fc-fragment of IgG subtype IgG4 incorporating the following mutations (S228P, F234A, L235A, K447-, Eu-numbering) (as in SEQ ID No.: 15), the complete sequence will be as in SEQ ID. No 28, also denominated as CCN5(dIII)-Fcv2.

In the case when the invention takes the embodiment of domain III of CCN5 genetically fused N-terminal of a peptide linker (as in SEQ ID. No 20) and an Fc-fragment of IgG subtype IgG4 incorporating the following mutations (S228P, E233P, F234V, L235A, G236-, K447-, Eu-numbering) as shown in SEQ ID. No 18, the resulting sequence will be as shown in SEQ ID. No 29, also denominated as CCN5(dIII)-Fcv2.1.

In the case when the invention takes the embodiment of domain III of CCN5 genetically fused N-terminal of a peptide linker (as in SEQ ID. No 20) and a chimeric Fc-fragment of IgG subtype IgG2/4 subtype, as shown in SEQ ID. No 19, the resulting sequence will be as shown in SEQ ID. No 30, also denominated as CCN5(dIII)-Fcv2.3.

In the case when the invention takes the embodiment of domain III of CCN5 (as in SEQ ID. No 1) genetically fused N-terminal of a peptide linker (as in SEQ ID. No 25) and a chimeric Fc-fragment of IgG subtype IgG2/4 subtype, as shown in SEQ ID. No 19, the resulting sequence will be as shown in SEQ ID. No 31, also denominated as CCN5(dIII)-HLn8-Fcv2.3.

According to a preferred embodiment of the invention, a fusion protein according to the present invention are provided comprising:
1) a point mutation into domain III of a CCN family protein, in particular CCN5 (cf. SEQ ID No. 7, resulting in reduced proteolytic susceptibility of said domain III;
2) an engineered chimera of the Fc-fragment of human IgG4 and human IgG2 (SEQ ID No. 19, which decreases proteolytic susceptibility relative to previously described Fc-fragment backbones utilized in Fc-fusion proteins; and
3) comprising an optimized composition of the peptide linker (cf. SEQ ID No. 21-25), which decreases proteolytic susceptibility, enhances biologic activity of the fusion protein and reduces aggregation tendency of the fusion protein.

In some embodiments, the peptide linker between the amino acid sequence of (i) and the monomeric fusion partner has an amino acid sequence selected from the group consisting of SEQ ID NOs: 20 to 25 or 39, or an amino acid sequence having 80% sequence identity thereto.

Alternative linker sequences which may be used according to the present invention are provided in SEQ ID NOs: 57, 63, 65, 67 and 121.

### Recombinant Expression

The recombinant proteins and fusion proteins according to the present invention may be manufactured by culturing a host cell enabling the expression of nucleotide sequences encoding said proteins. The skilled person is well familiar with the various available biotechnological techniques providing for the expression of isolated nucleic acid sequences for the preparation of recombinant proteins by heterologous expression in various host cell systems using commonly available genetic engineering techniques and recombinant DNA expression systems, cf. e.g. “Recombinant Gene Expression Protocols, in Methods in Molecular Biology, 1997, Ed. Rocky S Tuan, Human Press (ISSN 1064-3745) or Sambrook et al., Molecular Cloning: A laboratory Manual (third edition), 2001, CSHL Press, (ISBN 978-087969577-4). For example, the nucleic acid sequences encoding the recombinant proteins according to the present invention may be inserted in suitable expression vectors comprising all the necessary transcriptional and translational regulatory sequences specifically adapted for directing the expression of the desired protein coding nucleic acid sequence in a suitable host cell. Suitable expression vectors are e.g. plasmids, cosmids, viruses or artificial yeast chromosomes (YAC's).

DNA sequences encoding the recombinant proteins of the invention may be synthesized by methods well known to the skilled person or commercial suppliers well known to the skilled person, e.g. Genscript, Thermo Fisher Scientific etc.

According to one embodiment of this aspect, a DNA molecule is provided comprising a nucleic acid sequence as depicted in SEQ ID NO: 86, 87, 90, 91, 99, 100, 104, 105, 108, 109, 112, or 113, or a sequence having at least 80% sequence identity to any aforementioned sequence. Expression vectors comprising such DNA molecules are also provided. According to another embodiment of this aspect, host cells comprising such vectors are also provided.

DNA sequences to be expressed and used to prepare recombinant proteins may be inserted in vectors commonly known as entry vectors using the Gateway cloning system (Esposito et al, 2009, “Gateway Cloning for Protein Expression”, in Methods in Molecular Biology, 498, pp. 31-54). Genes cloned into an entry vector may easily be introduced into a variety of expression vectors by recombination. As an example, the synthesized sequence encoding a recombinant protein or fusion protein according to the present invention may be recombined by BP Gateway recombinase cloning to generate an Entry vector which may be used to propagate the plasmids in a suitable host cell, such as E. coli cells. In a preferred embodiment, E. coli cells mutated to allow for efficient propagation of plasmids is used, such as e.g. One Shot Top10™ cells.

According to one embodiment of the present invention, an expression vector is prepared comprising a DNA sequence encoding a recombinant protein or a fusion protein according to the present invention operably linked to a promotor. The skilled person will acknowledge that a “promoter” as used herein refers to a region of DNA upstream (5′-prime) of a DNA coding sequence that controls and initiates transcription of the particular gene. The promoter controls recognition and binding of RNA polymerase and other proteins to initiate transcription. “Operably linked” refers to a functional linkage between a promoter and a second sequence, where the promoter sequence initiates and mediates transcription of the DNA sequence corresponding to the second sequence. In general, operably linked means that the nucleic acid sequences being linked are contiguous.

The entry vector as well as an expression vector, such as that generated from a destination vector mentioned below, may be isolated using standard plasmid isolation techniques well known to the skilled person, such as e.g. using a QIAprep™ Spin Miniprep kit from Qiagen™ or QIAGEN™ Plasmid Plus Maxi Kit. If fusing an Entry vector containing a DNA sequence encoding a recombinant protein or fusion protein according to the present invention, said entry vector may be further recombined with a destination vector using LR gateway recombinase to generate an expression vector. The expression vector may then be used to express the protein coding DNA sequence in an appropriate host cell. Non-limiting examples of applicable destination vector is e.g. pUCOE-DHFR-DEST, as described in Kaasbell et al., J. Biol. Chem, 293:46, pp. 17953-17970.

Also, the resulting expression vector may be verified by standard restriction enzyme digestion and DNA gel electrophoresis.

According to one aspect of the invention, an expression vector is provided comprising a nucleic acid sequence encoding a recombinant protein of formula (I). According to yet another aspect of the invention, an expression vector is provided comprising a nucleic acid sequence encoding a protein comprising an amino acid sequence selected from the group consisting of SEQ ID No. 1, SEQ ID No. 2, SEQ ID No. 3, SEQ ID No. 4, SEQ ID No. 5, SEQ ID No. 6, SEQ ID No. 7, and SEQ ID NOs: 8-12, 37, 38, 84, 85, 88, 89, 97, 98, 102, 103, 106, 107, 110 and 111; and fragments or variants thereof having at least 50% sequence identity with the amino acid sequences SEQ ID No. 1, SEQ ID No. 2, SEQ ID No. 3, SEQ ID No. 4, SEQ ID No. 5, SEQ ID No. 6, and SEQ ID No. 7, SEQ ID No.8, SEQ ID No. 9, SEQ ID No. 10, SEQ ID No. 11, SEQ ID No. 12, SEQ ID No. 37 and SEQ ID No. 38, and, and SEQ ID NOs: 84, 85, 88, 89, 97, 98, 102, 103, 106, 107, 110 and 111.

According to another aspect, an expression vector is provided encoding a recombinant protein comprising an amino acid sequence having at least 60%, 70%, 80%, 90%, or 95% sequence identity with an amino acid sequence selected from the group consisting of SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 37, and SEQ ID No. 38,and SEQ ID NOs: 84, 85, 88, 89, 97, 98, 102, 103, 106, 107, 110 and 111.

According to yet an embodiment of the present invention, expressions vectors are provided encoding a fusion protein according to the present invention.

The skilled person is well aware of the degeneration of the genetic code, and the preference for specific codons in various organisms. Thus, dependent upon the selection of host cell, the nucleic acid sequence encoding the recombinant protein and fusion proteins of the invention may be adapted to the preferred codons of the host cell. Thus, the amino acids of the proteins of the invention may be encoded by any combination of the codons as shown in the table below:

| | Single Letter | |
|---|---|---|
| Amino Acid | Amino Acid Code | DNA codons |
| Isoleucine | I | ATT, ATC, ATA |
| Leucine | L | CTT, CTC, CTA, CTG, TTA, TTG |
| Valine | V | GTT, GTC, GTA, GTG |
| Phenylalanine | F | TTT, TTC |
| Methionine | M | ATG |
| Cysteine | C | TGT, TGC |
| Alanine | A | GCT, GCC, GCA, GCG |
| Glycine | G | GGT, GGC, GGA, GGG |
| Proline | P | CCT, CCC, CCA, CCG |
| Threonine | T | ACT, ACC, ACA, ACG |
| Serine | S | TCT, TCC, TCA, TCG, AGT, AGC |
| Tyrosine | Y | TAT, TAC |
| Tryptophan | W | TGG |
| Glutamine | Q | CAA, CAG |
| Asparagine | N | AAT, AAC |
| Histidine | H | CAT, CAC |
| Glutamic acid | E | GAA, GAG |
| Aspartic acid | D | GAT, GAC |
| Lysine | K | AAA, AAG |
| Arginine | R | CGT, CGC, CGA, CGG, AGA, AGG |
| Stop codons | Stop | TAA, TAG, TGA |

Preferably the codons are furthermore optimized for high expression according to the selected host cell.

For expression of proteins by recombinant DNA technology, in addition to the particular embodiment of the invention, a DNA sequence encoding a signal peptide is preferably appended to the N-terminal end of the protein sequence. The signal peptide may serve to direct localization of the fusion protein during and/or after synthesis in a host cell. It may accordingly be a sequence directing secretion of the fusion protein. The use of such signal peptide sequences is well known in the art. The signal peptide may take any form, e.g. it may constitute the IgGk-chain signal peptide, or it may constitute the signal peptide from human serum albumin (SEQ ID. No 32).

In the case when the signal peptide from human serum albumin (SEQ ID. No 32) is appended to the N-terminal of SEQ ID. No 28, the protein sequence to be expressed may be as shown in SEQ ID. No 33, or SEQ ID NOs: 85, 89, 98, 103, 107, or 111.

Furthermore, for expression of the protein by recombinant DNA technology, in according to one particular embodiment of the invention, a protein having an amino acid sequence as depicted in SEQ ID NO. 33, a nucleotide sequence as depicted in SEQ ID. No 34 or SEQ ID NOs: 86, 90, 99, 104, 108, or 112 may be used, wherein a translational stop codon is appended to the 3′ end of the coding sequence.

In the case when the invention is embodied by the nucleotide sequence in SEQ ID.No. 34, the nucleotide sequence is preferably appended to the immediate 5′-end of the coding sequence by a Kozak sequence, e.g. GCCACC, as in SEQ ID No. 35 or SEQ ID NOs: 86, 90, 99, 104, 108, or 112. The DNA sequence may further be flanked by DNA elements to enable subcloning, e.g. such as gateway recombinase attB sites. However, any cloning or synthesis strategy may be used to generate the DNA sequence and facilitate subcloning into an expression vector. In the case when the DNA sequence incorporates gateway recombinase sites to enable subcloning, the nucleotide sequence may be as shown in SEQ ID. No 36 or SEQ ID NOs: 87, 91, 100, 105, 109, or 113.

The obtained expression vector including a nucleic acid sequence encoding a recombinant protein of fusion protein of the invention may be introduced in suitable host cells for the production of the desired protein. Various commercially available or proprietary host cells may be used. For example, an expression vector may be transferred into eukaryote host cells, such as CHO cells, e.g. CHO DG44 DHFR (dihydrofolate reductase^{-/-}) suspension culture adapted CHO cells. Transfection of host cells with the expression vector may be performed by methods well known to the skilled person, e.g. using electroporation.

Upon culturing the host cells in a suitable culturing media, the recombinant proteins or fusion proteins according to the present invention encoded by the expression vector in the host cell will be produced, and the resulting protein may be collected and purified by methods well known to the skilled person.

The expression vector may include signal sequences, commonly known as “signal peptide”, for secretion of the expressed protein or fusion protein into the culture media.

For isolation and purification of the secreted recombinant protein from the cell culture medium, one or more pre-treatments or clarification steps is commonly used first in order to remove large particles and biomass. Non-limiting examples of applicable pre-treatment steps are e.g. reverse osmosis, centrifugation, filtration methods and diafiltration, or a combination thereof. The obtained protein is then commonly purified by one or more of a variety of chromatographic methods well known to the skilled person, e.g. by affinity chromatography, ion-exchange chromatography, mixed-mode chromatography, hydrophobic interaction chromatography, size exclusion chromatography or other chromatography techniques, or a combination thereof.

For example, a recombinant protein or fusion protein expressed by a suitable host cell may be purified using an affinity chromatography method, such as using MabSelect™ SuRe™ media, e.g. such as a 5 ml HiTrap MabSelect™ SuRe™ column mounted on an FPLC chromatography system, e.g. the BioRad NGC Discover™ 10 Pro system fitted with a 5 mm UV flow cell. After loading of the sample comprising the protein to be purified, the column is commonly washed one or more times with one or more applicable wash buffers, whereafter the protein is eluted using an applicable elution buffer. The obtained protein may be further purified using one or more of the chromatography methods listed above.

It should be understood that various modification may be introduced in the nucleic sequences encoding the recombinant proteins of the present invention utilizing techniques well known to the skilled person for example to facilitate expression. By the use of site directed mutagenesis, modification may be introduced to adapt the coding sequence to the desired host used to express the sequence and thus produce the recombinant protein. The skilled person is well aware of the fact of the presence of host specific codons, and that the adaption of a heterologous nucleic acid sequence to the host specific codons increase the expression efficiency as mentioned above. Other modifications may also be introduced, e.g. to facilitate isolation and purification, i.e. by adding a sequence coding for a peptide or protein useful for such purposes. Also, nucleic acid sequences coding signal peptide providing for secretion of the desired recombinant protein from the host cell may also be linked to the nucleic acid sequences of the present invention.

The present invention furthermore provides a host cell suitable for production of a recombinant protein or fusion protein according to the present invention. Various commercially available host cells specifically adapted for the production of recombinant proteins may be used, both prokaryotic host cells and eukaryotic host cells. Non-limiting examples of suitable host cells are e.g. CHO cells, HEK293 cells, Pichia pastoris cells, NS0 cells or E-coli cells.

Finally, the present invention also relates to thrombospondin type 1 repeat (TSP-1) homology domain of a CCN family protein and fusion protein comprising said TSP-1 repeat homology domain for use as a medicament for treatment or prevention of disorders by inhibiting or counteracting the cell signaling and cell physiological functions ascribed to CCN family proteins.

In one aspect, the present invention provides a protein, e.g. fusion protein, as defined herein for use in therapy.

In some aspects, the protein, e.g. fusion protein, may be for use in the treatment or prevention of fibrosis, or any condition exhibiting fibrosis (i.e. any fibrotic condition or disorder). The fibrosis may affect any tissue or organ, including for example, the lung, eye, heart, skeletal muscle, peritoneum, kidney, liver, pancreas, bile ducts, skin, blood vessels, or more systemic systems. In particular, the condition exhibiting fibrosis may be selected from pulmonary fibrosis, which may be of any etiology, including idiopathic pulmonary fibrosis, bronchopulmonary dysplasia, retinal fibrosis, diabetic retinopathy, age-related macular degeneration, retinal detachment, oxygen induced retinopathy, glaucoma, cardiac fibrosis, post-transplant graft fibrosis, cardiomyopathy associated fibrosis, muscular fibrosis, Duchenne muscular dystrophy, peritoneal fibrosis, diabetic nephropathy, chronic kidney disease (kidney fibrosis), acute kidney injury, tubulointerstitial fibrosis, chronic allograft nephropathy, liver fibrosis, non-alcoholic steatohepatitis, fatty liver disease, chronic pancreatitis, biliary fibrosis, keloids, scarring, systemic sclerosis, atherosclerosis, epidural fibrosis.

In the context of cardiac fibrosis, the conditions to be treated or prevented may include cardiac hypertrophy and heart failure with or without preserved ejection fraction.

In a further aspect, the present invention provides a protein, e.g. fusion protein, as defined herein for use in the treatment of an inflammatory or autoimmune disease. In some embodiments, the inflammatory disease is selected from rheumatoid arthritis, amyotrophic lateral sclerosis (ALS), inflammatory bowel disease, ulcerative colitis, Crohn's disease.

In a further aspect, the present invention provides a protein, e.g. fusion protein as defined herein for use in the treatment of cancer. It is known in this regard that 4-domain CCN proteins can both elicit oncogenic responses in isolated cancer cells as well as contribute to metastasis, chemoresistance and immunotherapy resistance by acting on cancer cells directly or the tumour stroma. The activity of the proteins herein in inhibiting the effect or activity of a 4-domain CCN protein accordingly provides a rationale for treating cancer. The cancer may be any malignant or pre-malignant neoplastic condition. It may be of any tissue or organ. In an embodiment the cancer may manifest as solid tumours. In another embodiment the cancer may be of or in the haemopoietic system. It may be a primary cancer or a secondary cancer, or metastasis. The cancer may thus be a cancer of the pancreas, breast, prostate, cervix, ovary, liver, bladder, brain, blood, bone, skin, lung or stomach. In some embodiments, the cancer is selected from pancreatic cancer, pancreatic ductal adenocarcinoma, breast cancer, prostate cancer, cervical cancer, ovarian carcinoma, liver cancer, hepatocellular carcinoma, urothelial bladder cancer, brain cancer, glioblastoma, acute lymphoblastic leukemia, osteosarcoma, melanoma, mesothelioma, gastric carcinoma, oral squamous cell carcinoma, oesophagal cancer, colorectal cancer, lung cancer.

In a further aspect, the present invention provides a protein, e.g. a fusion protein, as defined herein for use in the treatment of a metabolic disease. The metabolic disease may be, or may be associated with, insulin resistance or glucose intolerance. In some embodiments, the metabolic disease is selected from type 2 diabetes and metabolic syndrome.

The fusion protein of the present invention may also be use in methods of treatment of the conditions described above. Similarly, the fusion protein of the present invention may be used in methods of manufacture of a medicament for use in the treatment of the conditions described above.

### EXAMPLES

### Example 1

### Expression of a Fusion Protein According to the Invention

In this example, the providing of a fusion protein comprising amino acid 194-246 of CCN5 (SEQ ID. No. 1), fused N-terminal of a peptide linker (SEQ ID No. 20) and Fc-fragment of IgG, IgG4 subclass of SEQ ID NO. 15 (S228P, F234A, L235A, K447-, Eu-numbering) (CCN5(dIII)-Fcv2) (i.e. a fusion protein according to SEQ ID. No. 28) is described. The fusion protein was further appended with an N-terminal signal sequence originating from albumin of SEQ ID No. 32 and was expressed in mammalian cells as disclosed below.

The DNA sequence shown in sequence SEQ ID No. 36 was synthesized and sequence verified by a commercial supplier. The synthesized sequence was recombined with pDonrZeo by BP Gateway recombinase cloning to generate an Entry vector. Following transfection of competent E. coli mutated to allow for efficient propagation of plasmids (One Shot Top10™ cells), the entry vector was isolated with standard plasmid isolation techniques through use of a QIAprep™ Spin Miniprep kit from Qiagen™. Following plasmid isolation, the entry vector was verified by restriction enzyme digestion followed by DNA gel electrophoresis according to standard techniques well known to the skilled person.

The Entry vector containing sequence SEQ ID NO. 35 was further recombined with a destination vector using LR gateway recombinase. The destination vector used was pUCOE-DHFR-DEST, as described by Kaasbell et al., 2018, supra.

Following transfection of competent E. coli, mutated to allow for efficient propagation of plasmids (One Shot Top10™ cells), the expression vector was isolated with standard plasmid isolation techniques using a QIAGEN™ Plasmid Plus Maxi Kit. The resulting expression vector was verified by standard restriction enzyme digestion and DNA gel electrophoresis according to standard techniques well known to the skilled person. The resulting expression vector was then transferred into ExpiCHO suspension culture adapted CHO cells according to the “Max Titer” protocol supplied by the manufacturer of the Expifectamine™ CHO transfection kit (Gibco Cat. #: A29129) and as described briefly in Kaasbell et al., 2018, supra. The cells were sedimented 6 days after transfection by centrifugation at 4750 g for 20 minutes at 4° C. and the supernatant cell culture medium harvested. 0.1M PMSF in 100% isopropanol was added to a concentration of 1 mM and 0.5M EDTA was added to a concentration of 2 mM. Then, 96% ethanol was added to a final concentration of about 3%. 1M TrisHCl pH 7.4 was added to a final concentration of 25 mM prior to chromatographic purification.

The capture step of the purification was performed by affinity chromatography with a protein A chromatography media. The media used in this experiment was rProtein A FF (GE Healthcare). A 5 mL HiTrap™ rProtein A FF column (GE Healthcare) was used for purification of the expressed recombinant protein from 60 mL of cell culture medium harvested and supplemented as described, supra. The HiTrap™ rProtein A FF column was mounted on an FPLC chromatography system (BioRad NGC Discover™ 10 Pro system) fitted with a 5 mm UV flow cell and equilibrated with a buffer containing 25 mM TrisHCl pH 7.4, 25 mM NaCl and 3% ethanol. The harvested cell culture medium containing the recombinant protein was loaded with a sample pump at a speed of 2.5 ml/min, followed by washing with 6 column volumes of wash buffer (25 mM TrisHCl pH 7.4, 25 mM NaCl and 3% ethanol)) prior to elution with 0.1M NaCitrate, pH 3.0, in 3% ethanol. Eluate with a UV 280 nm absorbance exceeding 100 mAU was collected in fractions of 3 mL in low-protein binding tubes pre-filled with 1 mL 1M TrisHCl pH 9.0. The fraction containing the UV absorbance peak was concentrated to 500 μL with the use of a Vivaspin® 20 mL, 30 kDA MWCO concentration device. Following concentration, the sample was loaded into a sample loading loop on the FPLC chromatography system (BioRad NGC Discover™ 10 Pro system). The FPLC chromatography system was fitted with a Superdex® 200 Increase 10/300 GL column (GE Healthcare) equilibrated with 50 mM NaCl, 20 mM HEPES pH 7.0. The sample was injected, and the column perfused with the pre-equilibration buffer (50 mM NaCl, 20 mM HEPES 40 pH 7.0) at a flowrate of 0.25 mL/minute. The main UV 280 nm absorbance peak was found to contain the purified recombinant protein (CCN5(dIII)-Fcv2, SEQ ID No. 28). 10 μL samples of the collected fractions were subjected to SDS-PAGE utilizing Mini-PROTEAN® TGX Stain-Free™ precast gels and the isolated recombinant proteins were visualized utilizing a ChemiDocm imaging system (BioRad).

It is widely known to the skilled person that recombinant proteins may be produced in various expressions systems and purified by a variety of chromatographic methods with similar results.

### Example 2

A DNA sequence encoding a fusion protein comprising amino acid 194-246 of CCN5 (SEQ ID. No. 1), fused N-terminal of a peptide linker (SEQ ID No. 20) and an Fc-fragment of IgG, subclass IgG4 of SEQ ID NO. 15 (S228P, F234A, L235A, K447-, Eu-numbering) (CCN5(dIII)-Fcv2) was expressed in order to produce a recombinant protein according to SEQ ID NO. 28.

The obtained protein was tested for its ability to inhibit prosurvival signaling (Serine-473 phosphorylation of AKT) in A549 human lung cancer cells (FIG. 1A).Tissue cultured treated Corning Incorporated Costar® 96 well sterile polystyrene plates were coated with fibronectin (Sigma Cat #F1141 diluted to 10 μg/mL in BioWhittaker® Dulbecco's phosphate buffered saline (Lonza Cat. No. 17-512F, hereafter referred to as PBS)). The coating solution containing fibronectin was distributed to the wells at a volume of 100 μL/well, incubated for 1 hour at room-temperature followed by decantation of the coating solution, 100 μL of PBS was also distributed to the fibronectin coated wells, which was also decanted. A549 cells sub-cultured to maintain a density of maximum 80% confluency were detached by enzymatic treatment (Accutase®, Cat No. L0950-100 from Biowest®), diluted in Dulbecco's Modified Eagle Medium with high glucose (Gibco Cat. No.: 41965-039) supplemented with 10% heat-inactivated Fetal Bovine Serum (FBS) (500 mL flasks with FBS (Cat. No. 16000-044 from gibco) equilibrated to room-temperature were incubated in a water bath with a temperature of 60° C. with shaking for 30 minutes) and 50 μg/ml gensumycin (Sanofi)) to a concentration of 110000 cells/mL and 100 μL of the cell solution was distributed to the fibronectin coated wells. All cell incubations were done in cell culture incubators maintaining a temperature of 37° C., a humid atmosphere of room-air and 5% CO2. Following overnight incubation, the A549 cells were washed twice in PBS and 90 μL Dulbecco's Modified Eagle Medium with high glucose (DMEM, Gibco Cat. No.: 41965-039) and 50 μg/ml gensumycin (Sanofi) without FBS was distributed to the wells. Following 18 hours incubation in medium without FBS the cells were stimulated with 10 μL solution of the recombinant protein in question. After stimulation for 60 minutes the medium was decanted, and cells harvested by adding 50 μL lysis buffer with blocking reagent as per supplied by the Cisbio Phospho-AKT (Ser473) kit (Cisbio Inc, Cat. No.: 64AKSPEG). Following addition of the lysis buffer with blocking reagent the 96-well plate is incubated for 60 min on a PST-60HL plus (ThermoFisher) plate shaker at 500 rpm. Following shaking, the lysed samples were triturated prior to the transfer of 16 μL from each well to white-walled HTRF 96 well low volume plates (Cisbio Inc., Cat. #: 66PL96025). To assay the amount of phosphorylated AKT (Ser473) 4 μL of a mix of labeled antibodies (50/50 vol/vol mix of phospho-AKT d2 and phospho-AKT Cryptate from the Cisbio Inc, Cat. No.: 64AKSPEG) was added to each well (to the negative control well only the cryptate antibody was used), the plates were sealed with adhesive plastic film and incubated at 4° C. overnight prior to reading at a PolarStar Omega plate reader (BMG Labtech, Germany) fitted with a TR-FRET recording head and 337 nm emission and 615 nm and 665 nm excitation filters. The ratio between the 665 nm and 615 nm excitation recordings were blank corrected and the values of the recombinant protein stimulated wells expressed as percentage of vehicle stimulated wells.

### Example 3

A DNA sequence encoding a fusion protein comprising amino acid 194-246 of CCN5 (SEQ ID. No. 1), fused N-terminal of a peptide linker (SEQ ID No. 20) and an Fc-fragment of IgG, subclass IgG4 of SEQ ID NO. 15 (S228P, F234A, L235A, K447-, Eu-numbering) (CCN5(dIII)-Fcv2) was expressed in order to produce a recombinant protein according to SEQ ID NO. 28.

The obtained protein was tested for its ability for inhibiting pro-fibrotic TGF-β-stimulated transcription (from SMAD2/3 binding cis-elements) in IMR90 human lung fibroblasts. (FIG. 1D). The assay was performed technically as described in Kaasbell et al. (2018) supra, with the exception of the utilization of 2500 IMR90 lung fibroblasts/well in place of Rat2 cells. The proteins used for stimulation were as indicated in FIG. 1D. The IMR90 cells were sub-cultured as described for the A549 cells prior to use, supra. The IMR90 cells were used prior to passage 20, i.e. prior to reaching replicative senescence.

### Example 4

A DNA sequence encoding a fusion protein comprising amino acid 194-246 of CCN5 (SEQ ID. No. 1), fused N-terminal of a peptide linker (SEQ ID No. 20) and an Fc-fragment of IgG, subclass IgG4 of SEQ ID NO. 15 (S228P, F234A, L235A, K447-, Eu-numbering) (CCN5(dIII)-Fcv2) was expressed in order to produce a recombinant protein according to SEQ ID NO. 28.

The obtained protein was tested for its ability for inhibiting proliferation of the human lung fibroblast cell line IMR90 (FIG. 1B). The IMR90 cells were sub-cultured as described for the A549 cells prior to use, supra. The IMR90 cells were used prior to passage 20, i.e. prior to reaching replicative senescence. For the experiments the IMR90 cells were harvested as described for the A549 cells, supra, washed in PBS, diluted in DMEM with 1% FBS with gensumycin as described for Experiment 2, supra, and seeded in xCELLigence impedance plates at a density of 12000/well. After 2 hours the cells were stimulated with 10 μL solution of the recombinant protein in question or FBS and incubated for a further 72 hours before harvesting with CellTiter-Glo® (Promega Inc.) as described in Kaasbell et al., (2018), supra.

### Example 5

A DNA sequence encoding a fusion protein comprising amino acid 194-246 of CCN5 (SEQ ID. No. 1), fused N-terminal of a peptide linker (SEQ ID No. 20) and an Fc-fragment of IgG, subclass IgG4 of SEQ ID NO. 15 (S228P, F234A, L235A, K447-, Eu-numbering) (CCN5(dIII)-Fcv2) was expressed in order to produce a recombinant protein according to SEQ ID NO. 28.

The obtained protein was tested for its ability for inhibiting the sphere-forming ability (anchorage-independent growth) of the estrogen receptor-positive breast cancer cell line MCF-7 and of the triple-negative breast cancer cell line MDA-MB-231 (FIG. 1C) as described in Kaasbell et al., supra. The MDA-MB-231 cells were treated the same as described for the MCF-7 cell line in Kaasbell et al., supra. The MCF-7 and MDA-MB-231 cell lines were sub-cultured as described for the A549 cell line, supra.

### Example 6

DNA sequences encoding a fusion protein comprising amino acid 194-246 of CCN5 (SEQ ID. No. 1), fused N-terminal of a peptide linker (SEQ ID No. 20) and either the Fc-fragment of IgG, subclass IgG4 of SEQ ID NO. 15 (S228P, F234A, L235A, K447-, Eu-numbering) (CCN5(dIII)-Fcv2), the Fc-fragment of IgG, subclass IgG4 of SEQ ID No. 18 (S228P, E233P, F234V, L235A, G236-, K447-, Eu-numbering) (CCN5(dIII)-Fcv2.1) or a chimeric Fc-fragment of IgG2/4 subclasses (SEQ ID No. 19) (CCN5(dIII)-Fcv2.3) were expressed in order to produce recombinant protein according to SEQ ID NO. 28 (CCN5(dIII)-Fcv2), SEQ ID NO. 29 (CCN5(dIII)-Fcv2.1) and SEQ ID NO. 30 (CCN5(dIII)-Fcv2.3).

Specifically, expression vectors coding for the expression of SEQ ID NO. 28 (CCN5(dIII)-Fcv2), SEQ ID NO. 29 (CCN5(dIII)-Fcv2.1) and SEQ ID NO. 30 (CCN5(dIII)-Fcv2.3) were transfected into ExpiCHO suspension culture adapted CHO cells according to the “Max Titer” protocol supplied by the manufacturer of the Expifectamine™ CHO transfection kit (Gibco Cat. #: A29129) and as described briefly in Kaasbell et al., supra. The cells were sedimented 6 days after transfection by centrifugation at 13000 rpm in an Heraeus biofuge pico benchtop centrifuge for 5 minutes and the supernatant cell culture medium harvested. Samples of the harvested cell culture supernatants were separated by SDS-PAGE utilizing Mini-PROTEAN® TGX Stain-Free™ precast gels and the recombinant proteins were visualized utilizing a ChemiDoc™ imaging system (BioRad). The separated proteins were proteins were then transferred to PVDF-membranes using the Trans-Blot Turbo, semi-dry blotting system (Bio-Rad) for Western-blot analysis. The blot was probed with an anti-human IgG4 antibody conjugated to horseradish peroxidase (Invitrogen Cat. #: A10654) that was used in conjunction with SuperSignal™ West Femto Maximum Sensitivity Substrate (ThermoFisherScientific) and a ChemiDoc™ imaging system (BioRad) for visualization.

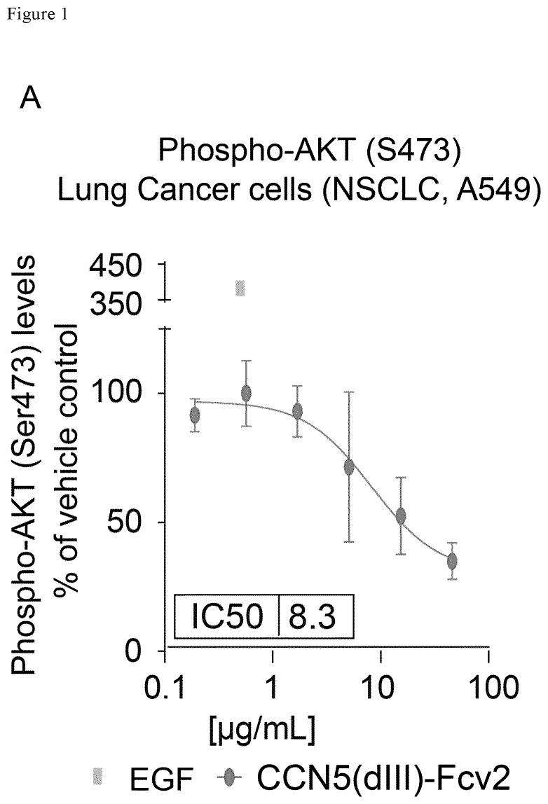

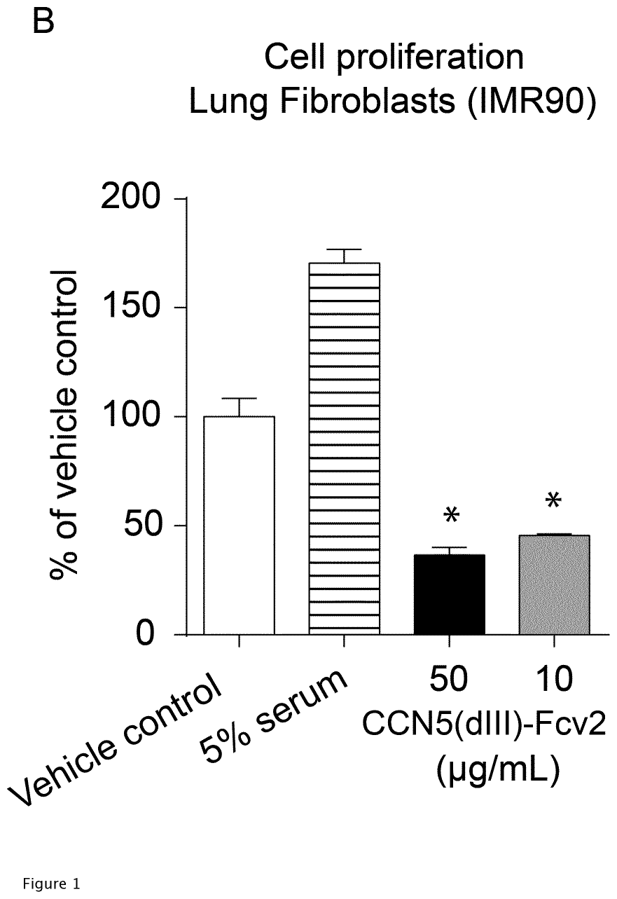

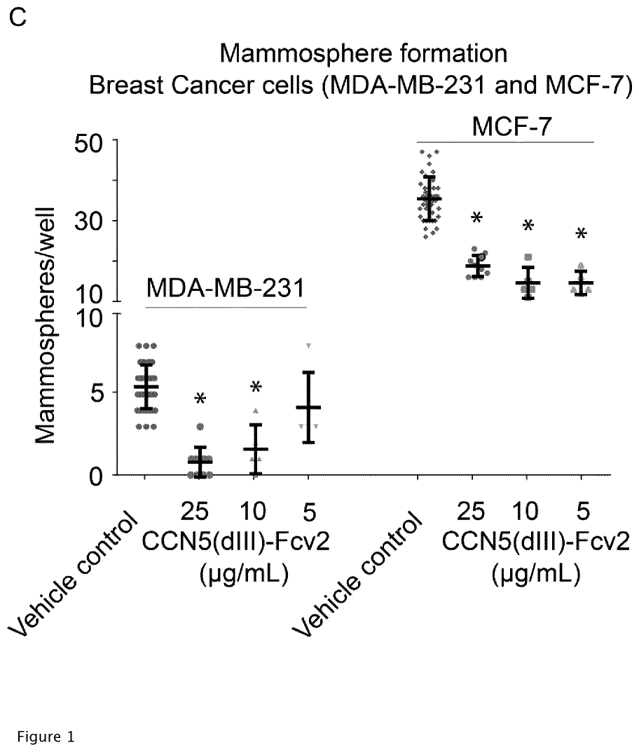

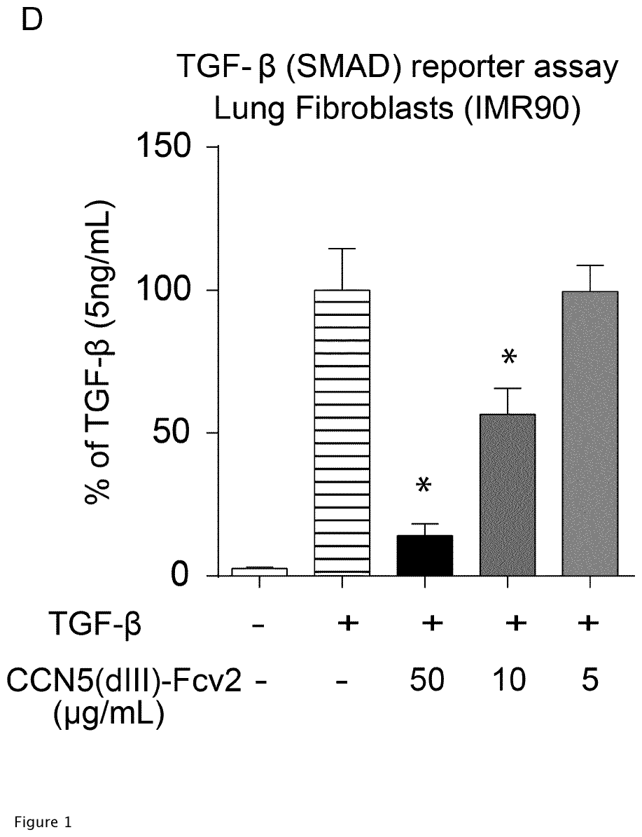

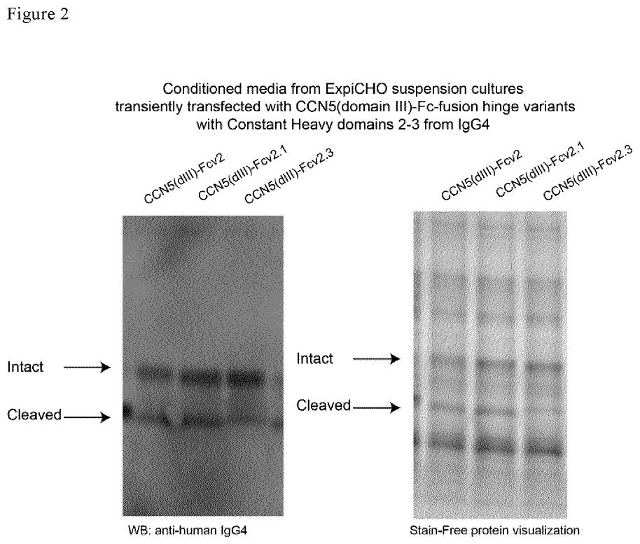

In FIG. 2, data demonstrating the improved protease resistance of the Fc-fragment backbone composed of the IgG2/4 chimera (shown in SEQ ID No. 19) are shown.

CCN5/WISP2(domain III) fused to IgG4 Fc-fragment with either an immune-effector silenced IgG4 hinge (as defined in SEQ ID No. 28); CCN5(domain III)-Fcv2, the same IgG4 backbone incorporating mutations based on IgG2 (as defined in SEQ ID. No. 29); CCN5(domain III)-Fcv2.1, or the same IgG4 backbone with a complete hinge region from IgG2 (as defined in SEQ ID No. 30); CCN5(domain III)-Fcv2.3 were expressed in the ExpiCHO system and conditioned medium (CM) was harvested after 6 days. Western blotting and total protein staining of SDS-PAGE gels reveals that the CCN5(domain III)-Fcv2.3 variant is the least susceptible to the proteases present during cultivation. Notice that the immunoreactivity of the anti-IgG4 antibody towards the Fc-fragment is partially lost with the substitution of sequences from IgG2, and thus underestimates the protein levels relative to the general protein staining.

### Example 7

DNA sequences encoding a fusion protein comprising amino acid 194-246 of CCN5 (SEQ ID. No. 1), fused N-terminal of either the peptide linker described in SEQ ID No. 20 and a chimeric Fc-fragment of IgG2/4 subclasses (SEQ ID No. 19). (CCN5(dIII)-Fcv2.3) or the peptide linker described in SEQ ID No. 25 and a chimeric Fc-fragment of IgG2/4 subclasses (SEQ ID No. 19) (CCN5(dIII)-HLn8-Fcv2.3) were expressed in order to produce recombinant protein according to SEQ ID NO. 30 (CCN5(dIII)-Fcv2.3) and SEQ ID No. 31 (CCN5(dIII)-HLn8-Fcv2.3).

Specifically, expression vectors coding for the expression of SEQ ID NO. 30 (CCN5(dIII)-Fcv2.3) and SEQ ID No. 31 (CCN5(dIII)-HLn8-Fcv2.3) were transfected into ExpiCHO suspension culture adapted CHO cells according to the “Max Titer” protocol supplied by the manufacturer of the Expifectamine™ CHO transfection kit (Gibco Cat. #: A29129) and as described briefly in Kaasbell et al., supra. The cells were sedimented 4 days after transfection by centrifugation at 13000 rpm in an Heraeus biofuge pico benchtop centrifuge for 5 minutes and the supernatant cell culture medium harvested. Samples of the harvested cell culture supernatant were separated by SDS-PAGE utilizing Mini-PROTEAN® TGX Stain-Free™ precast gels. The separated proteins were proteins were transferred to PVDF-membranes using the Trans-Blot Turbo, semi-dry blotting system (Bio-Rad) for Western-blot analysis. The blot was probed with an anti-human IgG4 antibody conjugated to horseradish peroxidase (Invitrogen Cat. #: A10654) that was used in conjunction with SuperSignal™ West Femto Maximum Sensitivity Substrate (ThermoFisherScientific) and a ChemiDoc™ imaging system (BioRad) for visualization.

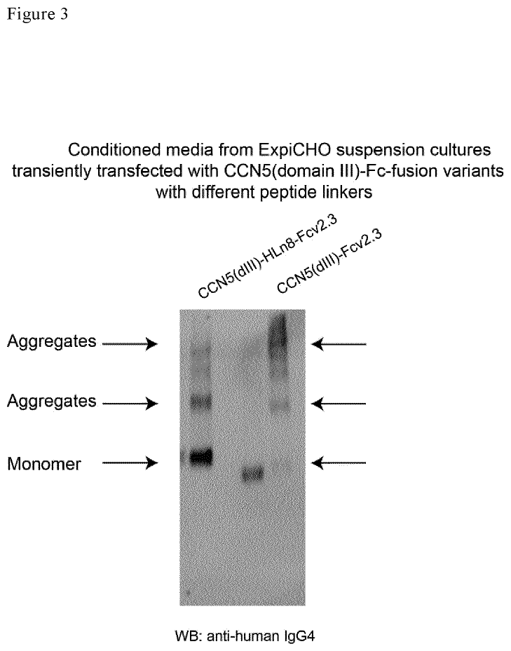

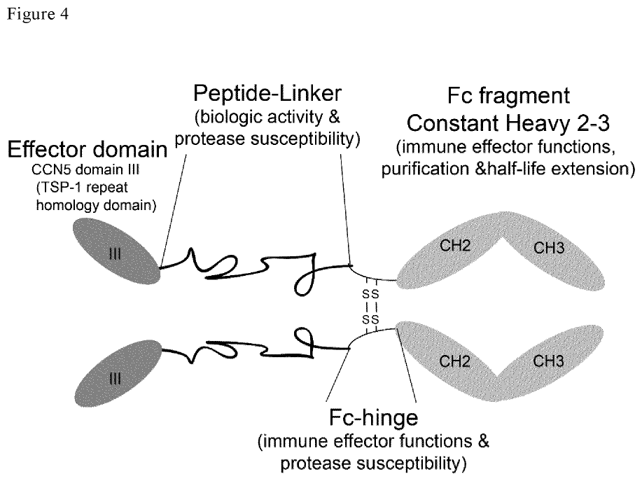

In FIG. 3 data showing the reduced tendency to aggregation when the embodiment of the invention incorporates a peptide linker as depicted in SEQ ID NO. 25 is provided.

Non-reducing SDS-PAGE of CM from transiently transfected CHO suspension cells expressing CCN5 (domain III) fused to the amino-terminal end of the chimeric IgG2/4 Fc-fragment through various peptide linkers. The Western blot reveals that fusion protein with an amino sequence as depicted SEQ ID No. 31; (dIII)-HLn8-Fcv2.3 has lower tendency to aggregate than a fusion protein of the invention having an amino acid sequence as depicted in SEQ ID No. 30; CCN5(domain III)-Fcv2.3. This finding demonstrates that the peptide linker defined in sequence SEQ ID No. 25 affords lower tendency of aggregation of the fusion protein compared with the fusion protein containing the peptide linker defined in sequence SEQ ID No. 20.

### Example 8

DNA sequences encoding a fusion protein comprising either amino acid 194-246 of CCN5 (SEQ ID. No. 1) or amino acids 194-246 of CCN5 (SEQ ID. No. 7), where amino acid in position 195 (proline) is substituted with alanine, fused C-terminal of a peptide linker (SEQ ID No. 39) and an Fc-fragment of IgG subtype IgG4 of SEQ ID NO. 15 (S228P, F234A, L235A, K447-, Eu-numbering) were expressed in order to produce recombinant protein according to SEQ ID No.: 40 (Fc-HLn8-CCN5(dIII)) or SEQ ID No.: 41 (Fc-HLn8-CCN5(dIII)-P195A).

Specifically, expression vectors coding for the expression of SEQ ID NO. 40 (Fc-HLn8-CCN5(dIII)) and SEQ ID No.: 41 (Fc-HLn8-CCN5(dIII)-P195A) were transfected into ExpiCHO suspension culture adapted CHO cells according to the “Max Titer” protocol supplied by the manufacturer of the Expifectamine™ CHO transfection kit (Gibco Cat. #: A29129) and as described briefly in Kaasbell et al., supra. The cells were sedimented 3 days after transfection by centrifugation at 13000 rpm in an Heraeus biofuge pico benchtop centrifuge for 5 minutes and the supernatant cell culture medium harvested. Samples of the harvested cell culture supernatant were separated by SDS-PAGE utilizing Mini-PROTEAN® TGX Stain-Free™ precast gels. The separated proteins were proteins were transferred to PVDF-membranes using the Trans-Blot Turbo, semi-dry blotting system (Bio-Rad) for Western-blot analysis. The blot was probed with an anti-human IgG4 antibody conjugated to horseradish peroxidase (Invitrogen Cat. #: A10654) that was used in conjunction with SuperSignal™ West Femto Maximum Sensitivity Substrate (ThermoFisherScientific) and a ChemiDoc™ imaging system (BioRad) for visualization.

In FIG. 5, data is presented showing the reduced susceptibility to endopeptidase cleavage when the embodiment of the invention incorporates a mutation of Proline 195 of the CCN5 TSP-1 repeat homology domain, as depicted in SEQ ID No 7.

Reducing SDS-PAGE of CM from transiently transfected CHO suspension cells expressing CCN5(domain III) fused to the carboxyl-terminal end of an IgG4 Fc-fragment as described in SEQ ID No. 15 either incorporating a P195A mutation (Fc-HLn8-CCN5(dIII)-P195A) or expressing the wild-type P195 variant of the CCN5 TSP-1 repeat homology domain (Fc-HLn8-CCN5(dIII)). This blot demonstrates that the P195A mutation affords the proteolytic resistance to the TSP-1 repeat homology domain of CCN5.

### Example 9

A fusion protein comprising amino acids 194-250 of human CCN5 (SEQ ID No. 56), fused N-terminal of a peptide linker (SEQ ID No. 57) and Fc-fragment of human IgG, IgG4 subclass of SEQ ID NO. 15 (S228P, F234A, L235A, K447-, Eu-numbering) resulting in a protein sequence corresponding to SEQ ID No. 58 (CCN5(dIII)-SL-Fcv0) is disclosed. The fusion protein was further appended with an N-terminal signal sequence for secretion originating from albumin of SEQ ID No. 32 to generate a fusion protein corresponding to SEQ ID No. 59 and was expressed in mammalian cells as disclosed below.

A DNA sequence of encoding the fusion protein of SEQ ID No. 59 was codon optimized for protein expression in hamster cells (by the algorithm of the commercial supplier), and a KOZAK sequence for translation was appended at the 5′ end and a STOP-codon was introduced at the 3′ end resulting in a DNA sequence of SEQ ID. No. 60. The DNA sequence was further appended at both ends by Gateway attB sites resulting in a DNA sequence of SEQ ID No. 61. The sequence of SEQ ID No. 61 was synthesized and verified by a commercial supplier. The synthesized sequence was recombined with pDonrZeo by BP Gateway recombinase cloning to generate an Entry vector. Following transfection of competent E. coli mutated to allow for efficient propagation of plasmids (One Shot Top10™ cells), the entry vector was isolated with standard plasmid isolation techniques through use of a QIAprep™ Spin Miniprep kit from Qiagen™. Following plasmid isolation, the entry vector was verified by restriction enzyme digestion followed by DNA gel electrophoresis according to standard techniques well known to the skilled person.

The Entry vector containing sequence SEQ ID No. 60 was further recombined with a destination vector using LR gateway recombinase. The destination vector used was pUCOE-DHFR-DEST, as described by Kaasbell et al., 2018, J. Biol. Chem, 293:46, pp. 17953-17970.

Following transfection of competent E. coli, mutated to allow for efficient propagation of plasmids (One Shot Top10™ cells), the expression vector was isolated with standard plasmid isolation techniques using a QIAGEN™ Plasmid Plus Maxi Kit. The resulting expression vector was verified by standard restriction enzyme digestion and DNA gel electrophoresis according to standard techniques well known to the skilled person. The resulting expression vector was then transfected into ExpiCHO cells adapted for suspension culture according to the “Max Titer” protocol supplied by the manufacturer of the Expifectamine™ CHO transfection kit (Gibco Cat. #: A29129) and as described briefly in Kaasbell et al., 2018, supra. The cells were sedimented 4 days after transfection by centrifugation at 4750×g for 20 minutes at 4° C. and the supernatant cell culture medium harvested. 0.1M PMSF in 100% isopropanol was added to a concentration of 1 mM and 0.5M EDTA was added to a concentration of 2 mM. Then, 96% ethanol was added to a final concentration of about 3%. 1M TrisHCl pH 7.4 was added to a final concentration of 25 mM prior to chromatographic purification.

The protein was purified by affinity chromatography utilizing protein A chromatography media. The chromatography media used in this experiment was rProtein A FF (GE Healthcare). A 5 mL HiTrap™ rProtein A FF column (GE Healthcare) was used for purification of the expressed recombinant protein from 120 mL of cell culture medium harvested and supplemented as described, supra. TheHiTrap™ rProtein A FF column was mounted on an FPLC chromatography system (BioRad NGC Discover™ 10 Pro system) fitted with a 5 mm UV flow cell and equilibrated with a buffer containing 25 mM TrisHCl pH 7.4, 25 mM NaCl and 3% ethanol. The harvested cell culture medium containing the recombinant protein was loaded with a sample pump at a speed of 2.5 ml/min, followed by washing with 10 column volumes of wash buffer (25 mM TrisHCl pH 7.4, 25 mM NaCl and 3% ethanol)) prior to elution with 0.1M NaCitrate, pH 3.0, in 3% ethanol. Eluted fractions of 3 mL were collected in in low-protein binding tubes pre-filled with 1 mL 1M TrisHCl pH 9.0. Protein elution was monitored with 280 nm UV absorbance and 10 μL samples of the pooled fractions containing the UV 280 nm absorbance peak were subjected to SDS-PAGE utilizing Mini-PROTEAN® TGX Stain-Free™ precast gels in the presence or absence of the reducing agent β-mercaptoethanol and the isolated recombinant proteins were visualized utilizing a ChemiDoc™ imaging system (BioRad).

It is widely known to the skilled person that recombinant proteins may be produced in various expressions systems and purified by a variety of chromatographic methods with similar results.

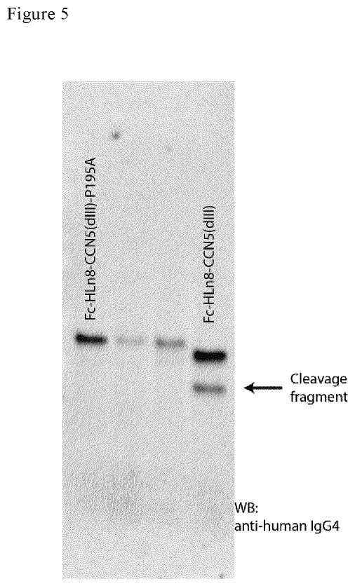

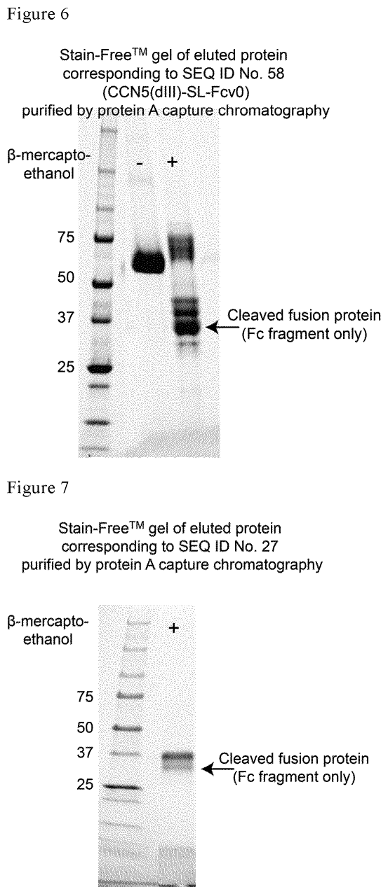

In FIG. 6 it is shown that the expression and purification of a protein corresponding to SEQ ID No. 58 does result in a protein that migrates higher than expected in the absence of the reducing agent β-mercaptoethanol, thus indicating dimer formation. However, as can be seen from the lane which contains the purified protein in the presence of the reducing agent β-mercaptoethanol, the expression and purification of the protein corresponding to SEQ ID No. 58 results primarily in cleavage fragments and not intact protein.

### Example 10

Multiple variants of the sequence of SEQ ID No. 58 were generated in attempt to increase the proteolytic resistance of the protein corresponding to SEQ ID No. 58. DNA sequences were synthesized and verified by a commercial supplier before being sub-cloned to generate plasmids as described in example 9 and the proteins expressed as described in example 9. The variants included proteins with modifications as listed below:
1) an N-terminal signal sequence originating from albumin of SEQ ID No. 32amino-terminal to fragment of CCN5 comprised of amino acids 194 to 249 incorporating mutation (P245L) corresponding to SEQ ID No. 62 combined with truncation of the peptide linker corresponding to SEQ ID No. 63 and the Fc fragment of SEQ ID No. 15 resulting in a sequence corresponding to SEQ ID No.64,
2) an N-terminal signal sequence originating from albumin of SEQ ID No. 32 amino-terminal to fragment of CCN5 comprised of amino acids 194 to 246 corresponding to SEQ ID No. 1 combined with a variation of the peptide linker corresponding to SEQ ID No. 65 and the Fc fragment of SEQ ID No. 15 resulting in a sequence corresponding to SEQ ID No. 66,
3) an N-terminal signal sequence originating from albumin of SEQ ID No. 32 amino-terminal to fragment of CCN5 comprised of amino acids 194 to 246 corresponding to SEQ ID No. 1 combined with a variation of the peptide linker corresponding to SEQ ID No. 67 and the Fc fragment of SEQ ID No. 15 resulting in a sequence corresponding to SEQ ID No. 68,
4) an N-terminal signal sequence originating from albumin of SEQ ID No. 32 amino-terminal to fragment of CCN5 comprised of amino acids 194 to 246 corresponding to SEQ ID No. 1 combined with a variation of the peptide linker corresponding to SEQ ID No. 65 and the Fc fragment of SEQ ID No. 19 resulting in a sequence corresponding to SEQ ID No. 69.

These iterations (1-4, supra) of the protein disclosed in Example 12 did show some improvement in their resistance to proteolytic cleavage during expression in the ExpiCHO system, performed as described in Example 9. However, expression of proteins corresponding to SEQ ID No. 64, SEQ ID. No 66, SEQ ID No. 68 and SEQ ID 69 revealed that the degree of proteolytic resistance was still insufficient to allow for the production of intact purified proteins.

### Example 11

A fusion protein was generated comprising amino acids 194-237 of CCN5 where the amino acid in position 195 (proline) is substituted with alanine (SEQ ID No. 38), fused N-terminal of a peptide linker (SEQ ID No. 21) and a chimeric Fc-fragment of IgG subtype IgG2/4 with deletion of the carboxyl-terminal K477- (Eu numbering) (SEQ ID No. 19) resulting in SEQ ID No. 27. The fusion protein was further appended with an N-terminal signal sequence for secretion originating from albumin of SEQ ID No. 32 to generate a fusion protein corresponding to SEQ ID No. 70. A DNA sequence of encoding the fusion protein of SEQ ID No. 70 was codon optimized for protein expression in hamster cells (by the algorithm of the commercial supplier), and a KOZAK sequence for translation was appended at the 5′ end and a STOP-codon was introduced at the 3′ end resulting in a DNA sequence of SEQ ID No. 71. The DNA sequence was further appended at both ends by Gateway attB sites resulting in a DNA sequence of SEQ ID No. 72.

DNA sequences were synthesized and verified by a commercial supplier before being sub-cloned to generate plasmids as described in example 9 and the protein corresponding to SEQ ID No. 70 expressed by transient transfection of ExpiCHO cells as described in example 9.

The cells were sedimented 6 days after transfection by centrifugation at 4750×g for 20 minutes at 4° C. and the supernatant cell culture medium harvested. 0.1M PMSF in 100% isopropanol was added to a concentration of 0.1 mM. 1M NaCitrate pH 5.5 was added to a final concentration of 30 mM prior to chromatographic purification.

The protein was purified by tandem-chromatography composed of a capture step with a 1 mL HiTrap™ MabSelectSuRe™ column (GE Healthcare) immediately followed by desalting with a BioScale™ Mini Bio-Gel® P-6 10 mL column (BioRad). The columns were mounted on an FPLC chromatography system (BioRad NGC Discover™ 10 Pro system) fitted with a 5 mm UV flow cell. The MabSelectSuRe™ column was mounted on the first column switching valve and equilibrated with a buffer composed of 30 mM NaCitrate pH 5.5 while the Bio-Gel® column was mounted on the second column switching valve and equilibrated with buffer A2 (100 mM NaH₂PO4/Na₂HPO4 pH 6.5). With the second column switching valve containing the Bio-Gel® column set to be bypassed 140 mL of the harvested cell culture medium containing the recombinant protein was loaded onto the MabSelectSuRe™ column with a sample pump at a speed of 2.0 ml/min, followed by washing with 5 column volumes of wash buffer A1 (30 mM NaCitrate pH 5.5), followed by 5 column volumes of wash buffer A3 (30 mM NaCitrate, 0.5M NaCl, pH 5.5), followed by 3 column volumes of wash buffer A1. Prior to elution with the elution buffer (30 mM Citric acid pH 3.4) the Bio-Gel® column mounted on the second column switching valve was switched to enter the flow-path. After eluting with 2 mL elution buffer the MabSelectSuRe™ column was switched out of the flow-path and the purified protein was eluted from the Bio-Gel® column with buffer A2. Protein elution was monitored with 280 nm UV absorbance and collection triggered once the absorbance exceeded 100 mAU. The collected fractions were pooled and a 10 μL sample was subjected to SDS-PAGE utilizing Mini-PROTEAN® TGX Stain-Free™ precast gels in the presence of the reducing agent β-mercaptoethanol and the isolated recombinant proteins were visualized utilizing a ChemiDoc™ imaging system (BioRad).

In FIG. 7 it is shown that the expression and purification of a protein corresponding to SEQ ID No. 27 in which the carboxyl-terminal tail of CCN5 is truncated is substantially more proteolytically resistant than variants in which all the carboxyl-terminal amino acids of CCN5 are included (as in SEQ ID No. 58, 64, 66, 68 and 69), even though the cell culture medium was harvested an additional 2 days after sub-cultivation relative to Example 9 (FIG. 6).

### Example 12

A fusion protein was generated comprising amino acids 206-249 of CCN3 where the amino acid in position 207 (isoleucine) is substituted with alanine (SEQ ID No. 44), fused N-terminal of a peptide linker (SEQ ID No. 21) and a chimeric Fc-fragment of IgG subtype IgG2/4 with deletion of the carboxyl-terminal K477- (Eu numbering) (SEQ ID No. 19) resulting in a fusion protein of SEQ ID No. 73. The fusion protein was further appended with an N-terminal signal sequence for secretion originating from albumin of SEQ ID No. 32 to generate a fusion protein corresponding to SEQ ID No. 74. A DNA sequence of encoding the fusion protein of SEQ ID No. 74 was codon optimized for protein expression in hamster cells (by the algorithm of the commercial supplier), and a KOZAK sequence for translation was appended at the 5′ end and a STOP-codon was introduced at the 3′ end resulting in a DNA sequence of SEQ ID No. 75. The DNA sequence was further appended at both ends by Gateway attB sites resulting in a DNA sequence of SEQ ID No. 76.

DNA sequences were synthesized and verified by a commercial supplier before being sub-cloned to generate plasmids as described in example 9 and the protein corresponding to SEQ ID No. 74 was expressed by transient transfection of ExpiCHO cells as described in example 9.

The cells were sedimented 5 days after transfection by centrifugation at 4750×g for 20 minutes at 4° C. and the supernatant cell culture medium harvested. 0.1M PMSF in 100% isopropanol was added to a concentration of 0.1 mM. 1M NaCitrate pH 5.5 was added to a final concentration of 30 mM prior to chromatographic purification.

The protein was purified by tandem-chromatography composed of a capture step with a 5 mL HiTrap™ MabSelectSuRe™ column (GE Healthcare) immediately followed by desalting with a 53 mL HiPrep™ 26/10 DeSalting column (GE Healthcare). The columns were mounted on an FPLC chromatography system (BioRad NGC Discover™ 10 Pro system) fitted with a 5 mm UV flow cell. The MabSelectSuRe™ column was mounted on the first column switching valve and equilibrated with a buffer composed of 30 mM NaCitrate pH 5.5 while the HiPrep™ column was mounted on the second column switching valve and equilibrated with buffer A2 (100 mM NaH₂PO4/Na₂HPO4 pH 6.5). With the second column switching valve containing the HiPrep™ column set to be bypassed 260 mL of the harvested cell culture medium containing the recombinant protein was loaded onto the MabSelectSuRe™ column with a sample pump at a speed of 3.5 ml/min, followed by washing with 5 column volumes of wash buffer A1 (30 mM NaCitrate pH 5.5), followed by 5 column volumes of wash buffer A3 (30 mM NaCitrate, 0.5M NaCl, pH 5.5), followed by 2 column volumes of wash buffer A1. Prior to elution with the elution buffer (30 mM Citric acid pH 3.4) the HiPrep™ column mounted on the second column switching valve was set to enter the flow-path. After eluting with 10 mL elution buffer the MabSelectSuRe™ column was switched out of the flow-path and the purified protein was eluted from the HiPrep™ column with buffer A2. Protein elution was monitored by UV absorbance at 280 nm and collection triggered once the absorbance exceeded 100 mAU. The collected fractions were pooled and a 10 μL sample was subjected to SDS-PAGE utilizing Mini-PROTEAN® TGX Stain-Free™ precast gels in the presence or absence of the reducing agent β-mercaptoethanol and the isolated recombinant proteins were visualized utilizing a ChemiDoc™ imaging system (BioRad).

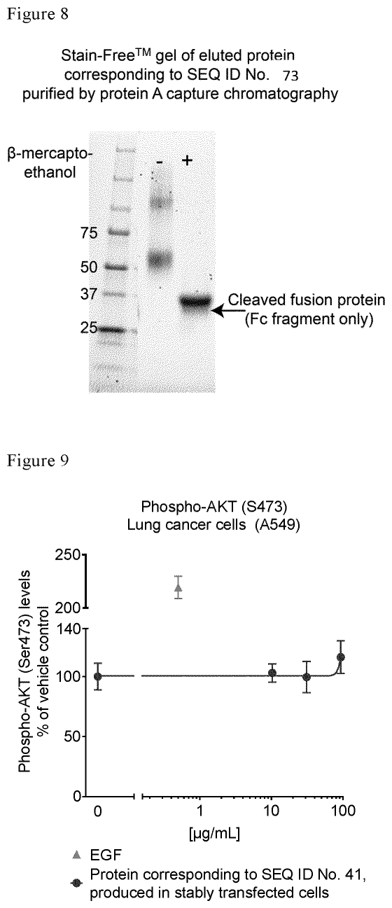

In FIG. 8 it can be seen that the fusion protein containing amino acids derived from CCN3/Nov (domain III/TSP-1 homology domain) as disclosed in SEQ ID No 73, analogous to the fusion protein containing amino acids derived from the homologous CCN5 (domain III/TSP-1 homology domain), as disclosed in SEQ ID No. 27, has similar or better resistance to proteolysis than the fusion protein containing amino acids derived from CCN5, as described in Example 11 and shown in FIG. 7.

### Example 13

A fusion protein comprising amino acids 194-246 of CCN5 where the amino acid in position 195 (proline) is substituted with alanine (SEQ ID No. 7), fused C-terminal of a peptide linker (SEQ ID No. 39) and an Fc-fragment of IgG, IgG4 subclass of SEQ ID NO. 15 (S228P, F234A, L235A, K447-, Eu-numbering) resulting in a protein sequence corresponding to SEQ ID No. 41, was appended by an N-terminal signal sequence for secretion originating from albumin of SEQ ID No. 32 resulting in a fusion protein corresponding to SEQ ID No. 77. A DNA sequence of encoding the fusion protein of SEQ ID No. 77 was codon optimized for protein expression in hamster cells (by the algorithm of the commercial supplier), and a KOZAK sequence for translation was appended at the 5′ end and a STOP-codon was introduced at the 3′ end resulting in a DNA sequence of SEQ ID No. 78. The DNA sequence was further appended at both ends by Gateway attB sites resulting in a DNA sequence of SEQ ID No. 79.

The sequence of SEQ ID No. 79 was synthesized and sequence verified by a commercial supplier. The synthesized sequence was recombined with pDonrZeo by BP Gateway recombinase cloning to generate an Entry vector. Following transfection of competent E. coli mutated to allow for efficient propagation of plasmids (One Shot Top10™ cells), the entry vector was isolated with standard plasmid isolation techniques through use of a QIAprep™ Spin Miniprep kit from Qiagen™. Following plasmid isolation, the entry vector was verified by restriction enzyme digestion followed by DNA gel electrophoresis according to standard techniques well known to the skilled person.

The Entry vector containing sequence SEQ ID No. 78 was further recombined with a destination vector using LR gateway recombinase. The destination vector used was pUCOE-DHFR-DEST, as described by Kaasbøll et al., 2018, J. Biol. Chem, 293:46, pp. 17953-17970.

Following transfection of competent E. coli, mutated to allow for efficient propagation of plasmids (One Shot Top10™ cells), the expression vector was isolated with standard plasmid isolation techniques using a QIAGEN™ Plasmid Plus Maxi Kit. The resulting expression vector was verified by standard restriction enzyme digestion and DNA gel electrophoresis according to standard techniques well known to the skilled person. The resulting expression vector was then transferred into suspension culture adapted DG44 CHO cells by means of electroporation utilizing the Neon transfection system (ThermoFisherScientific).

The cells were maintained in vented Erlenmeyer flasks in cell culture incubators kept at 37° C. with 8% CO₂ on a shaker platform (as described in Kaasbell et al., supra). The transfected cells were kept overnight in CD DG44 cell medium (Gibco, Cat. #12610-010) before being transferred to HyClone™ ActiPro™ medium (without hypoxanthine and thymidine, GE Healthcare) and sub-cultivated until viability approached 80%, at which point the medium was supplemented with 0.1 μM methotrexate. After the addition of 0.1 μM methotrexate the cells were sub-cultured until the viability again approached 80%, at which point the medium was supplemented with 1 μM methotrexate. The cells were again sub-cultured until the viability exceeded 98% and the doubling-time decreased to less than 26 hours, at which point the cell pool was considered stably transfected. Once the stable cell pool was established the cell culture volume was expanded to allow for the seeding of stably transfected cells for production at a density of 1*10\hat{ }6 cells/mL. The cell cultures were supplemented with 4/0.4% v/v HyClone™ Cell Boost™ 7a/7b every day from day 3 after sub-cultivation. After 10 days the cells were sedimented by centrifugation at 4750×g for 20 minutes at 4° C. and the supernatant cell culture medium harvested. 0.1M PMSF in 100% isopropanol was added to a concentration of 0.1 mM. 1M NaCitrate pH 5.5 was added to a final concentration of 30 mM prior to chromatographic purification.

The protein was purified by tandem-chromatography composed of a capture step with a 5 mL HiTrap™ MabSelectSuRe™ column (GE Healthcare) immediately followed by desalting with a 53 mL HiPrep™ 26/10 DeSalting column (GE Healthcare), as described in example 15. The purified protein preparation (which showed no signs of proteolytic processing) was subsequently tested for its ability to inhibit prosurvival signaling (Serine-473 phosphorylation of AKT) in A549 human lung cancer cells as described in Example 2. As can be seen from FIG. 9, the purified protein corresponding to SEQ ID No. 41 produced from the stably transfected pool of CHO suspension cells surprisingly showed no sign of a capacity to inhibit the phosphorylation of AKT (Serine 473).

### Example 14

A fusion protein was generated comprising amino acid amino acid 206-249 of CCN3 (SEQ ID. No. 44), where amino acid in position 207 (isoleucine) is substituted with alanine, fused N-terminal of a peptide linker (SEQ ID No. 22) and a chimeric Fc-fragment of IgG subtype IgG2/4 (SEQ ID No. 19) resulting in a protein sequence corresponding to SEQ ID No. 80, was appended by an N-terminal signal sequence for secretion originating from albumin of SEQ ID No. 32 resulting in a fusion protein corresponding to SEQ ID No. 81. A DNA sequence of encoding the fusion protein of SEQ ID No. 81 was codon optimized for protein expression in hamster cells (by the algorithm of the commercial supplier), and a KOZAK sequence for translation was appended at the 5′ end and a STOP-codon was introduced at the 3′ end resulting in a DNA sequence of SEQ ID No. 82. The DNA sequence was further appended at both ends by Gateway attB sites resulting in a DNA sequence of SEQ ID No. 83.

The sequence of SEQ ID No. 83 was synthesized and sequence verified by a commercial supplier. The synthesized sequence was recombined with pDonrZeo by BP Gateway recombinase cloning to generate an Entry vector. Following transfection of competent E. coli mutated to allow for efficient propagation of plasmids (One Shot Top10™ cells), the entry vector was isolated with standard plasmid isolation techniques through use of a QIAprep™ Spin Miniprep kit from Qiagen™. Following plasmid isolation, the entry vector was verified by restriction enzyme digestion followed by DNA gel electrophoresis according to standard techniques well known to the skilled person.

The Entry vector containing sequence SEQ ID No. 82 was further recombined with a destination vector using LR gateway recombinase. The destination vector used was pUCOE-DHFR-DEST, as described by Kaasbell et al., 2018, J. Biol. Chem, 293:46, pp. 17953-17970.

Following transfection of competent E. coli, mutated to allow for efficient propagation of plasmids (One Shot Top10™ cells), the expression vector was isolated with standard plasmid isolation techniques using a QIAGEN™ Plasmid Plus Maxi Kit. The resulting expression vector was verified by standard restriction enzyme digestion and DNA gel electrophoresis according to standard techniques well known to the skilled person. The resulting expression vector was then transferred into ExpiCHO suspension culture adapted CHO cells according to the “Creation and Scale up of a Stable Cell Line Using ExpiCHO™ Products” protocol supplied by the manufacturer of the ExpiCHO™ Stable Production Medium (Gibco Cat. #: A3711001). The cells were maintained in vented Erlenmeyer flasks in cell culture incubators kept at 37° C. with 8% CO₂ on a shaker platform (as described in Kaasbell et al., supra). The transfected cells were kept overnight in ExpiCHO™ Expression medium before being transferred to ExpiCHO™ expression medium supplemented with 0.1 μM methotrexate. The cells were then sub-cultured until the viability again approached 80%, at which point the medium was supplemented with 1p M methotrexate. The cells were again sub-cultured until the viability exceeded 95% and the doubling-time decreased to less than 20 hours, at which point the cell pool was considered stably transfected. Once the stable cell pool was established the cell culture volume was expanded to allow for the seeding of stably transfected cells for production at a density of 1*10\hat{ }6 cells/mL. After 5 days the cells were sedimented by centrifugation at 4750×g for 20 minutes at 4° C. and the supernatant cell culture medium harvested. 0.1M PMSF in 100% isopropanol was added to a concentration of 0.1 mM. 1M NaCitrate pH 5.5 was added to a final concentration of 30 mM and 2M L-Arginine pH 4.0 was added to a final concentration of 100 mM prior to chromatographic purification.

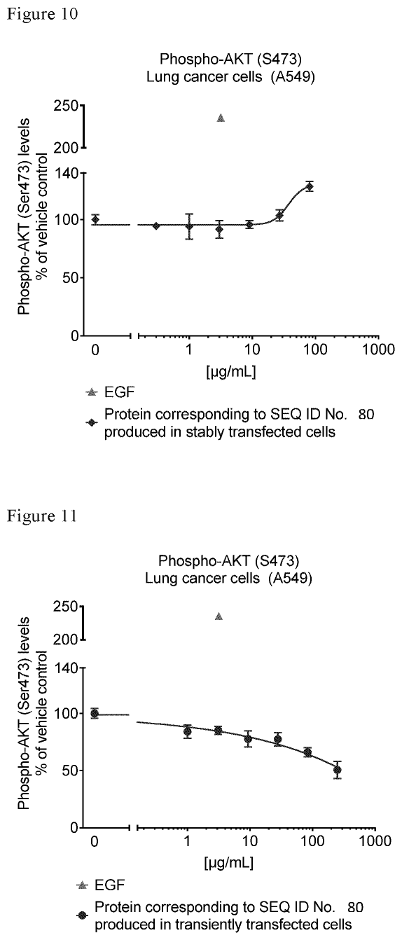

The protein was purified by tandem-chromatography composed of a capture step with a 5 mL HiTrap™ MabSelect Prism A™ column (GE Healthcare) immediately followed by desalting with a 53 mL HiPrep™ 26/10 DeSalting column (GE Healthcare), utilizing the same protocol as described in example 12 except for the addition of 100 mM L-Arginine to buffer A1, A2, A3 and B1. The purified protein preparation (which showed no signs of proteolytic processing) was subsequently tested for its ability to inhibit prosurvival signaling (Serine-473 phosphorylation of AKT) in A549 human lung cancer cells as described in Example 2. As can be seen from FIG. 10, the purified protein corresponding to SEQ ID No. 80 produced from the stably transfected pool of CHO suspension cells surprisingly showed no sign of a capacity to inhibit the phosphorylation of AKT (Serine 473) demonstrating that neither of the intact dimeric Fc-fusion protein preparations containing amino acids derived from CCN5 (SEQ ID No. 41, Example 13, FIG. 9) or amino acids derived from CCN3 (SEQ ID No. 80) are biologically active.

### Example 15

The expression plasmid described in example 14 containing SEQ ID No. 82, encoding for a fusion protein comprising amino acid amino acid 206-249 of CCN3 (SEQ ID. No. 44), where amino acid in position 207 (isoleucine) is substituted with alanine, fused N-terminal of a peptide linker (SEQ ID No. 22) and a chimeric Fc-fragment of IgG subtype IgG2/4 (SEQ ID No. 19) resulting in a protein sequence corresponding to SEQ ID No. 80, that is further appended by an N-terminal signal sequence for secretion originating from albumin of SEQ ID No. 32 and corresponding to SEQ ID No. 81, was expressed by transient transfection of ExpiCHO™ cells as described in example 9. The cells were sedimented, as described in example 14, 6 days after transfection and the media supplemented as described in example 14. The protein was purified by tandem-chromatography composed of a capture step with a 5 mL HiTrap™ MabSelect PrismA™ column (GE Healthcare) immediately followed by desalting with a 53 mL HiPrep™ 26/10 DeSalting column (GE Healthcare), as described in example 14.

The purified protein preparation (which was partially proteolytically processed) was subsequently tested for its ability to inhibit prosurvival signaling (Serine-473 phosphorylation of AKT) in A549 human lung cancer cells as described in Example 2.

As can be seen from FIG. 11, the purified protein corresponding to SEQ ID No.80 produced from the transiently transfected ExpiCHO™ cells displayed a concentration dependent capacity to inhibit the phosphorylation of AKT (Serine 473) demonstrating that the expression system used to produce the fusion protein corresponding to SEQ ID No. 80, and consequently the degree of proteolytic processing observed, greatly influences the activity, or lack thereof, of the resulting protein preparation.

### Example 16

A fusion protein comprising amino acid amino acid 206-249 of CCN3 (SEQ ID. No. 44), where amino acid in position 207 (isoleucine) is substituted with alanine, fused N-terminal of a peptide linker (SEQ ID No. 21) and an Fc fragment with monomer- inducing and half-life extending mutations (SEQ ID No. 55), resulting in a protein sequence corresponding to SEQ ID No. 84, was appended by an N-terminal signal sequence for secretion originating from albumin of SEQ ID No. 32 resulting in a fusion protein corresponding to SEQ ID No. 85. A DNA sequence encoding the fusion protein of SEQ ID No. 85 was codon optimized for protein expression in hamster cells (by the algorithm of the commercial supplier), and a KOZAK sequence for translation was appended at the 5′ end and a STOP-codon was introduced at the 3′ end resulting in a DNA sequence of SEQ ID No. 86. The DNA sequence was further appended at both ends by Gateway attB sites resulting in a DNA sequence of SEQ ID No. 86. DNA sequences were synthesized and verified by a commercial supplier before being sub-cloned to generate plasmids as described in example 9 and was expressed by transient transfection of ExpiCHO™ cells as described in example 9. The cells were sedimented, as described in example 14, 5 days after transfection and the media supplemented as described in example 14. The protein was purified by tandem-chromatography composed of a capture step with a 5 mL HiTrap™ MabSelect PrismA™ column (GE Healthcare) immediately followed by desalting with a 53 mL HiPrep™ 26/10 DeSalting column (GE Healthcare), as described in example 14.

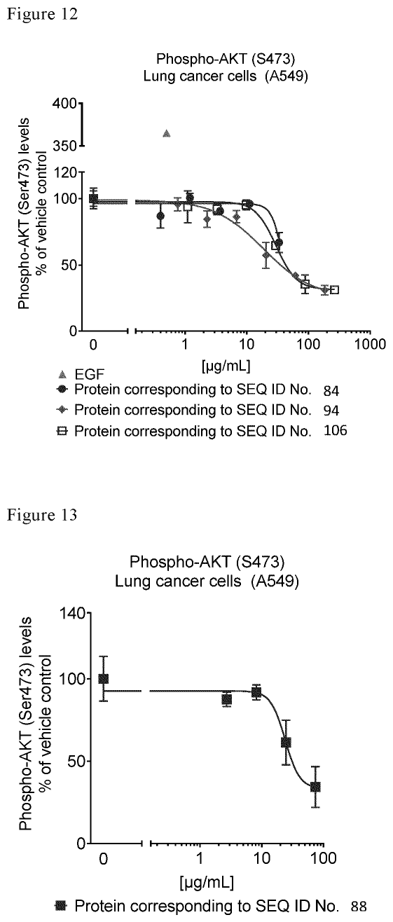

The purified protein preparation, which displayed the expected monomeric form, was subsequently tested for its ability to inhibit prosurvival signaling (Serine-473 phosphorylation of AKT) in A549 human lung cancer cells as described in Example 2. As can be seen from FIG. 12, the purified protein corresponding to SEQ ID No. 84 displayed a concentration dependent capacity to inhibit the phosphorylation of AKT (Serine 473) demonstrating that another monomeric fusion protein comprising amino acids from the domain III/TSP-1 homology domain of a CCN protein had the capacity to inhibit phosphorylation of AKT (Serine-473) in A549 human lung cancer cells.

### Example 17

A fusion protein comprising amino acid amino acid 206-249 of CCN3 (SEQ ID. No. 44), where amino acid in position 207 (isoleucine) is substituted with alanine, fused N-terminal of a peptide linker (SEQ ID No. 21) and an Fc fragment with monomer-inducing and stability inducing mutations (SEQ ID No. 54), resulting in a protein sequence corresponding to SEQ ID No. 88, was appended by an N-terminal signal sequence for secretion originating from albumin of SEQ ID No. 32 resulting in a fusion protein corresponding to SEQ ID No. 89. A DNA sequence of encoding the fusion protein of SEQ ID No. 89 was codon optimized for protein expression in hamster cells (by the algorithm of the commercial supplier), and a KOZAK sequence for translation was appended at the 5′ end and a STOP-codon was introduced at the 3′ end resulting in a DNA sequence of SEQ ID No. 90. The DNA sequence was further appended at both ends by Gateway attB sites resulting in a DNA sequence of SEQ ID No. 91. DNA sequences were synthesized and verified by a commercial supplier before being sub-cloned to generate plasmids as described in example 9 and was expressed by transient transfection of ExpiCHO™ cells as described in example 9. The cells were sedimented, as described in example 14, 6 days after transfection and the media supplemented as described in example 14. The protein was purified by tandem-chromatography composed of a capture step with a 5 mL HiTrap™ MabSelect PrismA™ column (GE Healthcare) immediately followed by desalting with a 53 mL HiPrep™ 26/10 DeSalting column (GE Healthcare), as described in example 14. The purified protein preparation, which predominantly displayed the expected monomeric form, was subsequently tested for its ability to inhibit prosurvival signaling (Serine-473 phosphorylation of AKT) in A549 human lung cancer cells as described in Example 2. As can be seen from FIG. 13, the purified protein corresponding to SEQ ID No. 88 displayed a concentration dependent capacity to inhibit the phosphorylation of AKT (Serine 473) demonstrating that another monomeric fusion protein comprising amino acids from the domain III/TSP-1 homology domain of a CCN protein had the capacity to inhibit phosphorylation of AKT (Serine-473) in A549 human lung cancer cells.

### Example 18

A fusion protein comprising amino acid amino acid 206-249 of CCN3 (SEQ ID. No. 44), where amino acid in position 207 (isoleucine) is substituted with alanine, fused N-terminal of a peptide linker (SEQ ID No. 93) and a multifunctional tag comprising 6×His tag, HaloTag and Sumo* elements (SEQ ID No. 92), resulting in a protein sequence corresponding to SEQ ID No. 94, was appended by an N-terminal signal sequence for secretion originating from albumin of SEQ ID No. 32 resulting in a fusion protein corresponding to SEQ ID No.114. A DNA sequence of encoding the fusion protein of SEQ ID No. 114 was codon optimized for protein expression in hamster cells (by the algorithm of the commercial supplier), and a KOZAK sequence for translation was appended at the 5′ end and a STOP-codon was introduced at the 3′ end resulting in a DNA sequence of SEQ ID No. 95. The DNA sequence was further appended at both ends by Gateway attB sites resulting in a DNA sequence of SEQ ID No. 96. DNA sequences were synthesized and verified by a commercial supplier before being sub-cloned to generate plasmids as described in example 9 and were expressed by transient transfection of ExpiCHO™ cells as described in example 15. The cells were sedimented, as described in example 14, 5 days after transfection. 0.1M PMSF in 100% isopropanol was added to a concentration of 0.1 mM, 1M NaCitrate pH 5.5 was added to a final concentration of 30 mM and 2M L-Arginine pH 4.0 was added to a final concentration of 0.1M and imidazole was added to a final concentration of 5 mM prior to chromatographic purification.

The protein was purified by tandem-chromatography composed of a capture step with a 5 mL HiTrap™ HisTrap™ excel column (GE Healthcare) immediately followed by desalting with a 53 mL HiPrep™ 26/10 DeSalting column (GE Healthcare). The columns were mounted on an FPLC chromatography system (BioRad NGC Discover™ 10 Pro system) fitted with a 5 mm UV flow cell. The HisTrap™ column was mounted on the first column switching valve and equilibrated with A1 buffer composed of 5 mM Imidazole, 50 mM NaCl, 100 mM L- Arginine while the HiPrep™ column was mounted on the second column switching valve and equilibrated with buffer A2 (100 mM NaH2PO4/Na2HPO4, 100 mM L-Arginine pH 6.5). With the second column switching valve containing the HiPrep™ column set to be bypassed 250 mL of the harvested cell culture medium containing the recombinant protein was loaded onto the HisTrap™ column with a sample pump at a speed of 3.5 ml/min, followed by washing with 5 column volumes of wash buffer A1, followed by 5 column volumes of wash buffer A3 (5 mM Imidazole, 0.5M NaCl, 100 mM L-Arginine), followed by 2 column volumes of wash buffer A1. Prior to elution with the elution buffer (250 mM Imidazole, 50 mM NaCl, 100 mM L-Arginine) the HiPrep™ column mounted on the second column switching valve was set to enter the flow-path. After eluting with 10 mL elution buffer the HisTrap™ column was switched out of the flow-path and the purified protein was eluted from the HiPrep™ column with buffer A2. Protein elution was monitored by UV absorbance at 280 nm and collection triggered once the absorbance exceeded 60 mAU.

The purified protein preparation, which displayed the expected monomeric form, was subsequently tested for its ability to inhibit prosurvival signaling (Serine-473 phosphorylation of AKT) in A549 human lung cancer cells as described in Example 2. As can be seen from FIG. 12, the purified protein corresponding to SEQ ID No. 94 showed a concentration dependent capacity to inhibit the phosphorylation of AKT (Serine 473) demonstrating that another monomeric fusion protein comprising amino acids from the domain III/TSP-1 homology domain of a CCN protein had the capacity to inhibit phosphorylation of AKT (Serine-473) in A549 human lung cancer cells.

### Example 19

A fusion protein comprising amino acids 194-237 of CCN5 where the amino acid in position 195 (proline) is substituted with alanine (SEQ ID No. 38), fused N-terminal of a peptide linker (SEQ ID No. 21) and amino acids 25-609 of human serum albumin (SEQ ID No. 52) resulting in SEQ ID No. 97 was appended by an N-terminal signal sequence for secretion originating from albumin of SEQ ID No. 32 resulting in a fusion protein corresponding to SEQ ID No. 98. A DNA sequence of encoding the fusion protein of SEQ ID No. 98 was codon optimized for protein expression in hamster cells (by the algorithm of the commercial supplier), and a KOZAK sequence for translation was appended at the 5′ end and a STOP-codon was introduced at the 3′ end resulting in a DNA sequence of SEQ ID No. 99. The DNA sequence was further appended at both ends by Gateway attB sites resulting in a DNA sequence of SEQ ID No. 100.

DNA sequences were synthesized and verified by a commercial supplier before being sub-cloned to generate plasmids as described in example 9 and DG44 CHO suspension cells engineered to express a constitutive active form of AKT were used to generate a stable pool of CHO suspension cells expressing the protein of SEQ ID No. 98 as described in Example 13. Once the stable cell pool was established the cell culture volume was expanded to allow for the seeding of stably transfected cells for production at a density of 1*10\hat{6} cells/mL. After 6 days the cells were sedimented by centrifugation at 4750×g for 20 minutes at 4° C. and the supernatant cell culture medium harvested. 0.1M PMSF in 100% isopropanol was added to a concentration of 0.1 mM, 0.5M EDTA was added to a final concentration of 2 mM, 1M NaCitrate pH 5.5 was added to a final concentration of 30 mM and 2M L- Arginine pH 4.0 was added to a final concentration of 0.1M prior to chromatographic purification.

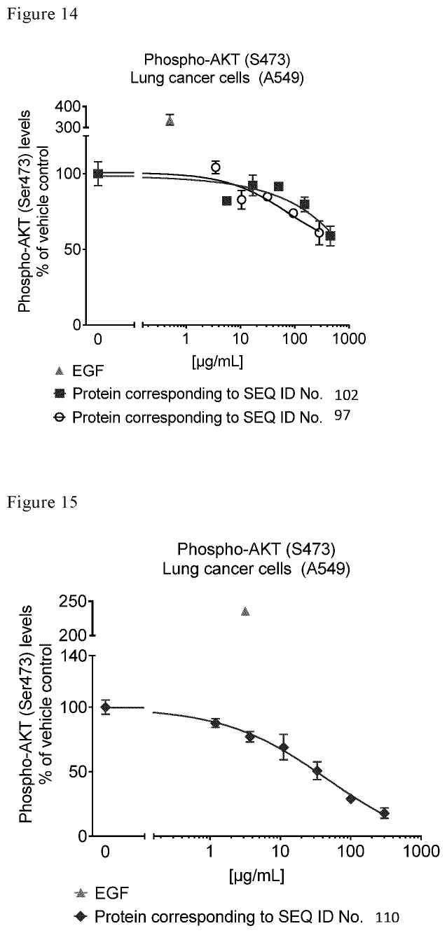

The protein was purified by tandem-chromatography composed of a capture step with a Tricorn column (GE Healthcare) packed with 3 mL of CaptureSelect™ Human Albumin Affinity Matrix (ThermoFisherScientific) immediately followed by desalting with a 53 mL HiPrep™ 26/10 DeSalting column (GE Healthcare). The columns were mounted on an FPLC chromatography system (BioRad NGC Discover™ 10 Pro system) fitted with a 5 mm UV flow cell. The CaptureSelect™-containing column was mounted on the first column switching valve and equilibrated with A1 buffer composed of 100 mM NaH2PO4/Na2HPO4, 100 mM L-Arginine pH 6.5 while the HiPrep™ column was mounted on the second column switching valve and equilibrated with buffer A1 (100 mM NaH2PO4/Na2HPO4, 100 mM L-Arginine pH 6.5). With the second column switching valve containing the HiPrep™ column set to be bypassed 500 mL of the harvested cell culture medium containing the recombinant protein was loaded onto the CaptureSelect™-containing column with a sample pump at a speed of 2.0 ml/min, followed by washing with 5 column volumes of wash buffer A1, followed by 5 column volumes of wash buffer A2 (100 mM NaH2PO4/Na2HPO4, 100 mM L-Arginine, 0.25M NaCl, pH 6.5), followed by 5 column volumes of wash buffer A1. Prior to elution with the elution buffer (30 mM Citric Acid, pH 3.5+0.5M L-arginine) the HiPrep™ column mounted on the second column switching valve was set to enter the flow-path. After eluting with 10 mL elution buffer the CaptureSelect™-containing column was switched out of the flow-path and the purified protein was eluted from the HiPrep™ column with buffer A1. Protein elution was monitored by UV absorbance at 280 nm and collection triggered once the absorbance exceeded 100 mAU. The purified protein preparation, which displayed the expected monomeric form, was subsequently tested for its ability to inhibit prosurvival signaling (Serine-473 phosphorylation of AKT) in A549 human lung cancer cells as described in Example 2. As can be seen from FIG. 14, the purified protein corresponding to SEQ ID No. 97 displayed a concentration dependent capacity to inhibit the phosphorylation of AKT (Serine 473) demonstrating that another monomeric fusion protein comprising amino acids from the domain III/TSP-1 homology domain of a CCN protein had the capacity to inhibit phosphorylation of AKT (Serine-473) in A549 human lung cancer cells.

### Example 20

A fusion protein of human serum albumin (amino acids 25-606, SEQ ID No. 101) was C-terminally fused to a peptide linker (SEQ ID NO. 22) connecting to amino acids 194-246 of human CCN5 where the amino acid in position 195 (proline) is substituted with alanine (SEQ ID No. 7), resulting in SEQ ID No. 103. The fusion protein corresponding to SEQ ID No. 102 was appended by an N-terminal signal sequence for secretion originating from albumin of SEQ ID No. 32 resulting in a fusion protein corresponding to SEQ ID No. 103. A DNA sequence of encoding the fusion protein of SEQ ID No. 103 was codon optimized for protein expression in hamster cells (by the algorithm of the commercial supplier), and a KOZAK sequence for translation was appended at the 5′ end and a STOP-codon was introduced at the 3′ end resulting in a DNA sequence of SEQ ID No. 104. The DNA sequence was further appended at both ends by Gateway attB sites resulting in a DNA sequence of SEQ ID No. 105.

DNA sequences were synthesized and verified by a commercial supplier before being sub-cloned to generate plasmids as described in example 9 and DG44 CHO suspension cells engineered to express a constitutive active form of AKT were used to generate a stable pool of CHO suspension cells expressing the protein of SEQ ID No. 104 as described in Example 13. Once the stable cell pool was established the cell culture volume was expanded to allow for the seeding of stably transfected cells for production at a density of 1*10\hat{ }6 cells/mL. After 6 days the cells were sedimented by centrifugation at 4750×g for 20 minutes at 4° C. and the supernatant cell culture medium harvested. 0.1M PMSF in 100% isopropanol was added to a concentration of 0.1 mM, 0.5M EDTA was added to a final concentration of 2 mM, 1M NaCitrate pH 5.5 was added to a final concentration of 30 mM and 2M L-Arginine pH 4.0 was added to a final concentration of 0.1M prior to chromatographic purification.

The protein was purified by tandem-chromatography composed of a capture step with a Tricorn column (GE Healthcare) packed with 3 mL of CaptureSelect™ Human Albumin Affinity Matrix (ThermoFisherScientific) immediately followed by desalting with a 53 mL HiPrep™ 26/10 DeSalting column (GE Healthcare) as described in Example 19 with the exception of the sample loading flow being 0.37 mL/min instead of 2.0 mL/min.

The purified protein preparation, which displayed the expected monomeric form, was subsequently tested for its ability to inhibit prosurvival signaling (Serine-473 phosphorylation of AKT) in A549 human lung cancer cells as described in Example 2. As can be seen from FIG. 14, the purified protein corresponding to SEQ ID No. 102 displayed a concentration dependent capacity to inhibit the phosphorylation of AKT (Serine 473) demonstrating that another monomeric fusion protein comprising amino acids from the domain III/TSP-1 homology domain of a CCN protein had the capacity to inhibit phosphorylation of AKT (Serine-473) in A549 human lung cancer cells.

### Example 21

A fusion protein comprising amino acids 206-249 of CCN3 where the amino acid in position 207 (isoleucine) is substituted with alanine (SEQ ID No. 44), fused N-terminal of a peptide linker (SEQ ID No. 21) and amino acids 25-609 of human serum albumin (SEQ ID No. 52) resulting in SEQ ID No. 106 was appended by an N-terminal signal sequence for secretion originating from albumin of SEQ ID No. 32 resulting in a fusion protein corresponding to SEQ ID No. 107. A DNA sequence of encoding the fusion protein of SEQ ID No. 107 was codon optimized for protein expression in hamster cells (by the algorithm of the commercial supplier), and a KOZAK sequence for translation was appended at the 5′ end and a STOP-codon was introduced at the 3′ end resulting in a DNA sequence of SEQ ID No. 108. The DNA sequence was further appended at both ends by Gateway attB sites resulting in a DNA sequence of SEQ ID No. 109.

DNA sequences were synthesized and verified by a commercial supplier before being sub-cloned to generate plasmids as described in example 9 and were expressed by transient transfection of ExpiCHO™ cells as described in example 9. The cells were sedimented, as described in example 14, 6 days after transfection and the media supplemented as described in example 19. The protein was purified by tandem-chromatography composed of a capture step with a Tricorn column (GE Healthcare) packed with 10 mL of CaptureSelect™ Human Albumin Affinity Matrix (ThermoFisherScientific) immediately followed by desalting with a 53 mL HiPrep™ 26/10 DeSalting column (GE Healthcare). The columns were mounted on an FPLC chromatography system (BioRad NGC Discover™ 10 Pro system) fitted with a 5 mm UV flow cell. The CaptureSelect™-containing column was mounted on the first column switching valve and equilibrated with A1 buffer composed of 100 mM NaH2PO4/Na2HPO4, 100 mM L-Arginine pH 6.5 while the HiPrep™ column was mounted on the second column switching valve and equilibrated with buffer A1 (100 mM NaH2PO4/Na2HPO4, 100 mM L-Arginine pH 6.5). With the second column switching valve containing the HiPrep™ column set to be bypassed 500 mL of the harvested cell culture medium containing the recombinant protein was loaded onto the CaptureSelect™-containing column with a sample pump at a speed of 1.0 ml/min, followed by washing with 3 column volumes of wash buffer A1, followed by 2 column volumes of wash buffer A2 (100 mM NaH2PO4/Na2HPO4, 100 mM L-Arginine, 0.25M NaCl, pH 6.5), followed by 3 column volumes of wash buffer A1. Prior to elution with the elution buffer (30 mM Citric Acid, pH 3.5+0.1M L-arginine) the HiPrep™ column mounted on the second column switching valve was set to enter the flow-path. After eluting with 15 mL elution buffer the CaptureSelect™-containing column was switched out of the flow-path and the purified protein was eluted from the HiPrep™ column with buffer A1. Protein elution was monitored by UV absorbance at 280 nm and collection triggered once the absorbance exceeded 100 mAU.

The purified protein preparation, which contained the expected monomeric form, was subsequently tested for its ability to inhibit prosurvival signaling (Serine-473 phosphorylation of AKT) in A549 human lung cancer cells as described in Example 2. As can be seen from FIG. 12, the purified protein corresponding to SEQ ID No. 106 displayed a concentration dependent capacity to inhibit the phosphorylation of AKT (Serine 473) demonstrating that another monomeric fusion protein comprising amino acids from the domain III/TSP-1 homology domain of a CCN protein had the capacity to inhibit phosphorylation of AKT (Serine-473) in A549 human lung cancer cells.

### Example 22

A fusion protein comprising amino acids 206-249 of CCN3 where the amino acid in position 207 (isoleucine) is substituted with alanine (SEQ ID No. 44), fused N-terminal of a peptide linker (SEQ ID No. 22) and amino acids 25-609 of human serum albumin (SEQ ID No. 52) resulting in SEQ ID No. 110 was appended by an N-terminal signal sequence for secretion originating from albumin of SEQ ID No. 32 resulting in a fusion protein corresponding to SEQ ID No. 111. A DNA sequence of encoding the fusion protein of SEQ ID No. 111 was codon optimized for protein expression in hamster cells (by the algorithm of the commercial supplier), and a KOZAK sequence for translation was appended at the 5′ end and a STOP-codon was introduced at the 3′ end resulting in a DNA sequence of SEQ ID No. 112. The DNA sequence was further appended at both ends by Gateway attB sites resulting ina DNA sequence of SEQ ID No. 113.

DNA sequences were synthesized and verified by a commercial supplier before being sub-cloned to generate plasmids as described in example 9 and was expressed by transient transfection of ExpiCHO™ cells as described in example 9. The cells were sedimented, as described in example 14, 6 days after transfection and the media supplemented as described in example 19. The protein was purified by tandem-chromatography composed of a capture step with a Tricorn column (GE Healthcare) packed with 10 mL of CaptureSelect™ Human Albumin Affinity Matrix (ThermoFisherScientific) immediately followed by desalting with a 53 mL HiPrep™ 26/10 DeSalting column (GE Healthcare). The columns were mounted on an FPLC chromatography system (BioRad NGC Discover™ 10 Pro system) fitted with a 5 mm UV flow cell. The CaptureSelect™-containing column was mounted on the first column switching valve and equilibrated with A1 buffer composed of 100 mM NaH2PO4/Na2HPO4, 100 mM L-Arginine pH 6.5 while the HiPrep™ column was mounted on the second column switching valve and equilibrated with buffer A1 (100 mM NaH2PO4/Na2HPO4, 100 mM L-Arginine pH 6.5). With the second column switching valve containing the HiPrep™ column set to be bypassed 300 mL of the harvested cell culture medium containing the recombinant protein was loaded onto the CaptureSelect™-containing column with a sample pump at a speed of 1.0 ml/min, followed by washing with 3 column volumes of wash buffer A1, followed by 2 column volumes of wash buffer A2 (100 mM NaH2PO4/Na2HPO4, 100 mM L-Arginine, 0.25M NaCl, pH 6.5), followed by 3 column volumes of wash buffer A1. Prior to elution with the elution buffer (30 mM Citric Acid, pH 3.5+0.5M L-arginine) the HiPrep™ column mounted on the second column switching valve was set to enter the flow-path. After eluting with 15 mL elution buffer the CaptureSelect™-containing column was switched out of the flow-path and the purified protein was eluted from the HiPrep™ column with buffer A1. Protein elution was monitored by UV absorbance at 280 nm and collection triggered once the absorbance exceeded 100 mAU.

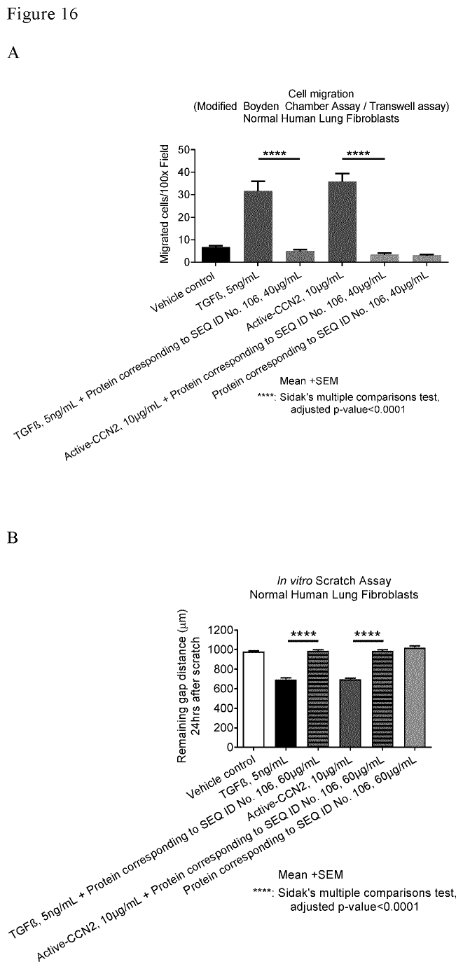

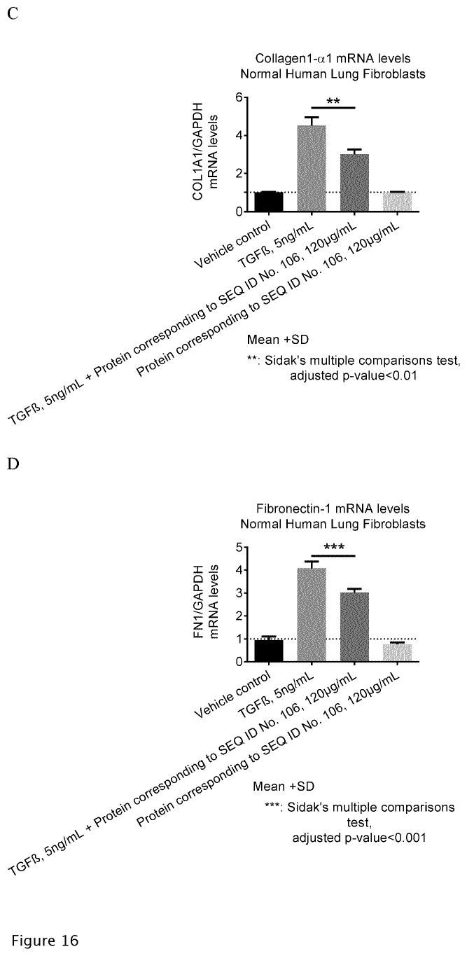

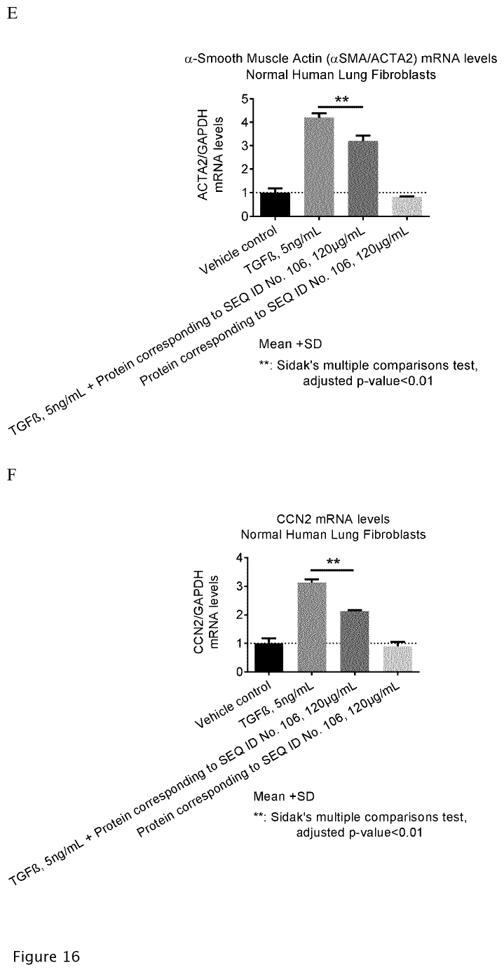

The purified protein preparation, which contained the expected monomeric form, was subsequently tested for its ability to inhibit prosurvival signaling (Serine-473 phosphorylation of AKT) in A549 human lung cancer cells as described in Example 2. As can be seen from FIG. 15, the purified protein corresponding to SEQ ID No. 110 displayed a concentration dependent capacity to inhibit the phosphorylation of AKT (Serine 473) demonstrating that another monomeric fusion protein comprising amino acids from the domain III/TSP-1 homology domain of a CCN protein had the capacity to inhibit phosphorylation of AKT (Serine-473) in A549 human lung cancer cells.

### Example 23

The expression plasmid described in example 21 containing SEQ ID No. 108, encoding for a fusion protein comprising amino acids 206-249 of CCN3 where the amino acid in position 207 (isoleucine) is substituted with alanine (SEQ ID No. 44), fused N-terminal of a peptide linker (SEQ ID No. 21) and amino acids 25-609 of human serum albumin (SEQ ID No. 52) that is further appended by an N-terminal signal sequence for secretion originating from albumin of SEQ ID No. 32 and corresponding to SEQ ID No. 107, was used to generate a pool of stably transfected ExpiCHO™ cells as described in example 14. To produce a batch of conditioned medium containing the secreted protein corresponding to SEQ ID NO. 106 the pool of stably transfected cells was expanded to allow for seeding of a volume of 250 mL of the stably transfected cells at a density of 1*10\hat{ }6 cells/mL. The cell cultures were supplemented with 5% v/v 2× EfficientFeed™ C+ (Gibco™) every other day starting from day 2 after sub-cultivation, and supplemented with 3% glucose (10% w/v) at day 2 after sub-cultivation and 5% glucose (10% w/v) at day 6 after sub-cultivation. After 9 days the cells were sedimented by centrifugation at 4750×g for 20 minutes at 4° C. and the supernatant cell culture medium harvested. 0.1M PMSF in 100% isopropanol was added to a concentration of 0.1 mM. 1M NaCitrate pH 5.5was added to a final concentration of 30 mM and 2M L-Arginine pH 4.0 was added to a final concentration of 100 mM prior to chromatographic purification.

The protein was purified by 2D-chromatography composed of a capture step with a Tricorn column (GE Healthcare) packed with 10 mL of CaptureSelect™ Human Albumin Affinity Matrix (ThermoFisherScientific) immediately followed by size exclusion chromatography with two serially connected Superdex 200 Increase 10/300 GL (GE Healthcare) columns. The columns were mounted on an FPLC chromatography system (BioRad NGC Discover™ 10 Pro system) fitted with a 5 mm UV flow cell and an outlet valve connected to a 5 mL sample loop. The CaptureSelect™-containing column was mounted on the first column switching valve and equilibrated with A1 buffer composed of 100 mM NaH2PO4/Na2HPO4, 100 mM L-Arginine pH 6.5 while the Superdex 200 Increase columns were mounted on the second column switching valve and equilibrated with buffer A1 (100 mM NaH2PO4/Na2HPO4, 100 mM L-Arginine pH 6.5). With the second column switching valve containing the Superdex 200 Increase columns set to be bypassed 120 mL of the harvested cell culture medium containing the recombinant protein was loaded onto the CaptureSelect™-containing column with a sample pump at a speed of 3.9 ml/min. After loading the harvested cell culture medium containing the recombinant protein onto the CaptureSelect™-containing column it was washed with 3 column volumes of buffer A1, followed by 2 column volumes of buffer A2 (100 mM NaH2PO4/Na2HPO4, 100 mM L-Arginine, 0.25M NaCl, pH 6.5), followed by 3 column volumes of buffer A1. The CaptureSelect™-containing column was eluted with 15 mL buffer B1 (30 mM citric acid, 0.5M L-Arginine, pH 3.5) during which the system was set to collect eluate with an absorbance exceeding 1200 mAU into the sample loop. Following elution of the CaptureSelect™-containing column, the CaptureSelect™-containing column connected to the first column switching valve was switched out of the flow path and the second column switching valve was set to switch the Superdex 200 Increase containing columns into the flow-path. The eluate from the CaptureSelect™-containing column containing the eluted protein was then loaded onto the Superdex 200 Increase containing columns with buffer Alat a speed of 0.5 mL/min. Protein elution was monitored by UV absorbance at 280 nm and collection triggered once the absorbance exceeded 200 mAU. The purified protein preparation, which contained the expected monomeric form, was subsequently tested for its ability to inhibit TGFβ-induced and active-CCN2 induced activities of normal human lung fibroblasts (NHLF) (Lonza Bioscience, Catalog #: CC-2512). The NHLFs were sub-cultured in complete growth medium (Lonza Bioscience bullet kit (Cat #: CC-3132) with all additives (2% fetal bovine serum, insulin, hFGF-B, gentamicin/amphotericin-B)) to maintain a density of maximum 80% confluency according to the commercial supplier's (Lonza Bioscience) instructions. Active-CCN2 was composed of domains 3-4 of CCN2 and produced and purified as described by Kaasbell et al., 2018, supra. To test the effect of the protein corresponding to SEQ ID No. 106 on the active-CCN2 and TGFβ-induced cell migration of NHLFs (transwell assay/modified Boyden chamber assay), the cells were first detached with Trypsin/EDTA, neutralized with Trypsin neutralization reagents (Lonza Bioscience, Cat. #CC-5034), and resuspended in basal growth media (Fibroblast basal media (LonzaBioscience Cat. #: CC-3131, without other additives than gensumycin (50 μg/mL))) before seeding 30 000 cells in a volume of 100 μL per well on the upper side of transwell inserts with a 5 μm pore-size (24-well plate, Corning® Transwell®, Cat. #CLS3402-48EA from Sigma Aldrich (Merck KGaA)). The lower chamber of the wells contained the test substances or vehicle control dissolved in 500 μL of the basal growth media without other additives than gensumycin. After 20 hours incubation the inserts were removed from the wells, washed twice by dipping in phosphate buffered saline (PBS, Lonza Bioscience, Cat. #: 17-512F) prior to fixation in 4% formaldehyde (Solveco, Swe., Cat. #: 621092) for 15 minutes at 37° C. The cells were permeabilized by treatment with 0.1% Triton X-100 in PBS for 10 minutes prior to washing twice with PBS. The non-migrated cells on the upper side of the inserts were removed by scraping with a cotton swap before the membrane was allowed to dry. The nuclei of the migrated cells on the underside of the insert were stained with Hoechst 33342 20 mM (1:5000 diluted in PBS, ThermoFisherScientific, Cat. #: 62249) for 15 minutes in the dark, prior to washing twice by dipping in PBS. The membrane was cut out from the transwell insert and the mounted on glass slides with the migrated cells towards the glass, covered with one drop of ProLong™ Gold Antifade (ThermoFisherScientific, Cat. #: P36934), mounted with a glass coverslip, and 5-10 images of each well were captured on a Zeiss Axio Observer Z.1 imaging system. Images were semi-automatically analyzed utilizing the ImageJ software v1.51k, Rasband, W. S., ImageJ, U. S. National Institutes of Health, Bethesda, Maryland, USA, https://imagej.nih.gov/ij/, 1997-2018.). As can be seen in FIG. 16A, the protein corresponding to SEQ ID No. 106 inhibits the migration induced by both TGFβ and active-CCN2.

To test the effect of the protein corresponding to SEQ ID No. 106 on the active-CCN2 and TGFβ-induced scratch-wound assay NHLFs were detached with Trypsin/EDTA, neutralized with Trypsin neutralization reagents (Lonza Bioscience, Cat. #CC-5034), before seeding 100 000 cells in a volume of 1 mL in tissue culture treated 12-well plates (Corning Costar®, Cat. #3513). The day after seeding, the cells were washed twice with 0.9% NaCl and the complete growth medium changed to basal growth medium. After having been incubated in the basal growth medium for 16-20 hours, a scratch was made in the cell monolayer with a sterile 12.5 μL pipette-tip (ThermoFisherScientific, Cat #: 94420053), the cells were washed once with PBS, before the cells were incubated in 1 mL of basal growth media together with test substances or vehicle. The cells were incubated for a further 24 hours before being washed three times in PBS before being fixed for 15 min at 37° C. in 4% formaldehyde. After fixation the cells were again washed for 3×3 minutes in PBS with gentle shaking, permeabilized with 0.1% Triton X-100 in PBS for 10 minutes with gentle shaking. The nuclei of the cells were stained by with Hoechst 33342 20 mM (1:5000 diluted in PBS, ThermoFisherScientific, Cat. #: 62249) for 15 minutes in the dark, prior to washing 3×5 minutes in PBS with gentle shaking. 1 drop of ProLong™ Gold Antifade (ThermoFisherScientific, Cat. #: P36934) was applied before mounting and 5 images centered on the remaining gap were captured from of each well with a Zeiss Axio Observer Z.1 imaging system. Images were analyzed by measuring the remaining gap distance after the scratch at 3 fixed intervals along the length of the scratch wound. The mean of all of the measurements from all of the images from each well was calculated and counted as one biological replicate. As can be seen in FIG. 16B, the protein corresponding to SEQ ID No. 106 inhibits the closure of the scratch wound induced by both TGFβ and active-CCN2.

To test the effect of the protein corresponding to SEQ ID No. 106 on TGFβ-induced gene regulation NHLFs were detached with Trypsin/EDTA, neutralized with Trypsin neutralization reagents (Lonza Bioscience, Cat. #CC-5034), before seeding 100 000 cells in a volume of 1 mL in tissue culture treated 12-well plates (Corning Costar®, Cat. #3513). The day after seeding, the cells were washed twice with 0.9% NaCl and the complete growth medium changed to basal growth medium supplemented with 0.1% heat-inactivated fetal bovine serum (Cat. #16000-044 from Gibco™, heat-inactivation performed as described in Example 2). After incubation in the basal growth medium with 0.1% fetal bovine serum for 6 hours the test substances or vehicle control were added to the wells. After 96 hours the wells were washed twice in PBS and RNA extracted utilizing the Qiagen RNeasy RNA extraction kit (Cat. #74106) according to the manufacturer's protocol. RNA concentrations were quantitated with a NanoDrop® ND-1000 spectrophotometer (NanoDrop Technologies, US), diluted with nuclease-free water to a final RNA concentrations of 50 ng/μL before 200 ng RNA from each replicate was utilized to generate cDNA by utilizing the TaqMan™ Reverse Trancription kit (Cat. #N8080234) according to the manufacturer's protocol. Differential gene expression analysis was analyzed from the resulting cDNA samples by means of respective TaqMan™ assays and the TaqMan Fast Advanced Master Mix (ThermoFisherScientific Cat. #4444557). The TaqMan™ real time PCR reactions were run with technical triplicates for each sample using Applied Biosystems StepOnePlus Real Time PCR System according to the manufacturers' protocols. Relative quantities of the different transcripts were calculated from a standard curve before the technical triplicates were averaged to yield a single value from each sample. All gene expression results were related to GAPDH (ThermoFisherScientific, Cat. #Hs02786624_g1) mRNA levels and normalized to be expressed as folds of the mean of the vehicle control-stimulated wells. As can be seen from FIG. 19C-F, the protein corresponding to SEQ ID No. 107 affords partial inhibition of the TGFβ-induced genes; COL1A1 (“collagen type 1 α-1”, ThermoFisherScientific, Cat. #Hs00164004_m1), FN1 (“fibronectin 1”, ThermoFisherScientific, Cat. #Hs01549976_m1), ΔCTA2 (“smooth muscle actin α-2”, ThermoFisherScientific, Cat. #Hs00426835_g1) and CCN2 (ThermoFisherScientific, Cat. #Hs00170014_m1), commonly regarded to be pro-fibrotic genes.

### Overview of the sequence numbers referred to in the specification and Sequence Listing

| SEQ | |
|---|---|
| ID | |
| No. | Sequence information |
| 1 | Amino acids 194-246 of human WISP2/CCN5 (domain III/TSP-1 |
| | homology domain) (long fragment) |
| 2 | Amino acids 206-249 of human NOV/CCN3 (domain III/TSP-1 homology |
| | domain) (44 aa fragment) |
| 3 | Amino acids 199-242 of human CTGF/CCN2 (domain III/TSP-1 |
| | homology domain) (44 aa fragment) |
| 4 | Amino acids 229-272 of human Cyr61/CCN1 (domain III/TSP-1 |
| | homology domain) (44 aa fragment) |
| 5 | Amino acids 216-259 of human WISP1/CCN4 (domain III/TSP-1 |
| | homology domain) (44 aa fragment) |
| 6 | Amino acids 209-252 of human WISP3/CCN6 (domain III/TSP-1 |
| | homology domain) (44 aa fragment) |
| 7 | Amino acids 194-246 of human WISP2/CCN5, (long fragment) where |
| | amino acid in position 195 (proline) is substituted with alanine |
| 8 | Amino acids 209-263 of human WISP3/CCN6 (domain III/TSP-1 |
| | homology domain) (long fragment) |
| 9 | Amino acids 206-258 of human NOV/CCN3 (domain III/TSP-1 homology |
| | domain) (long fragment) |
| 10 | Amino acids 199-250 of human CTGF/CCN2 (domain III/TSP-1 |
| | homology domain) (long fragment) |
| 11 | Amino acids 229-280 of human Cyr61/CCN1 (domain III/TSP-1 |
| | homology domain) (long fragment) |
| 12 | Amino acids 216-267 of human WISP1/CCN4 (domain III/TSP-1 |
| | homology domain) (long fragment) |
| 13 | Fc-fragment of IgG4 |
| 14 | Fc-fragment of IgG2 |
| 15 | Mutated Fc-fragment of IgG4 (S228P, F234A, L235A, K447-, cf. Eu- |
| | numbering) |
| 16 | Aglycosylated (N297G), disulfide bridge stabilized (R292C, V302C) Fc- |
| | fragment of IgG1, Eu numbering |
| 17 | Fc-fragment of IgG1, disulfide bridge stabilized (R292C, V302C), |
| | aglycosylated (N297G) and with protease stabilizing mutations in lower |
| | hinge region (E233P, L234V, L235A, G236-, Eu numbering) |
| 18 | Fc-fragment of IgG4, and with protease stabilizing mutations in lower |
| | hinge region (E233P, F234V, L235A, G236-, Eu-numbering) and with |
| | the S228P mutation and K447- deletion. |
| 19 | Fc-fragment being a chimera of the hinge region of IgG2 and the |
| | constant heavy domains 2 and 3 of IgG4 with a deletion of the |
| | carboxyl-terminal K477- (Eu numbering). |
| 20 | Linker (TEGRMD) |
| 21 | Linker (EAAAK) |
| 22 | Linker (EAAAKEAAAKEAAAKEAAAKEAAAKEAAAKEAAAKEAAAK) n = 8 |
| 23 | Linker (TAEAAAKEAAAKEAAAKEAAAKEAAAKEAAAKEAAAKEAAAK) |
| 24 | Linker (EAAAKEAAAKEAAAKEAAAKEAAAKEAAAKEAAAKEAAAKAAA) |
| 25 | Linker (TAEAAAKEAAAKEAAAKEAAAKEAAAKEAAAKEAAAKEAAAKAAA) |
| 26 | Fusion protein of the invention comprising the domain III of CCN5 (SEQ ID |
| | No. 37), coupled N-terminally of a linker of SEQ ID No. 21 that is further |
| | coupled N-terminal to the IgG2/4 Fc-fragment of SEQ ID NO. 19. |
| 27 | Fusion protein of the invention comprising the domain III of CCN5 (SEQ ID |
| | No. 38), wherein proline position 195 is replaced by alanine, coupled N- |
| | terminally of a linker of SEQ ID No. 21 that is further coupled N-terminal |
| | to the IgG2/4 Fc-fragment of SEQ ID NO. 19. |
| 28 | Fusion protein of the invention comprising amino acid 194-246 of CCN5 |
| | (SEQ ID. No. 1), fused N-terminal of a peptide linker (SEQ ID No. 20) and |
| | an Fc-fragment of IgG subtype IgG4 of SEQ ID NO. 15 (S228P, F234A, |
| | L235A, K447-, Eu-numbering) (CCN5(dIII)-Fcv2) |
| 29 | Fusion protein comprising amino acid 194-246 of CCN5 (SEQ ID. No. 1), |
| | fused N-terminal of a peptide linker (SEQ ID No. 20) and an Fc-fragment of |
| | IgG subtype IgG4 of SEQ ID No. 18 (S228P, E233P, F234V, L235A, G236-, |
| | K447-, Eu-numbering) (CCN5(dIII)-Fcv2.1) |
| 30 | Fusion protein comprising amino acid 194-246 of CCN5 (SEQ ID. No. 1), |
| | fused N-terminal of a peptide linker (SEQ ID No. 20) and a chimeric Fc- |
| | fragment of IgG subtype IgG2/4 subtype (SEQ ID No. 19) (CCN5(dIII)-Fcv2.3). |
| 31 | Fusion protein comprising amino acid 194-246 of CCN5 (SEQ ID. No. 1), |
| | fused N-terminal of a peptide linker (SEQ ID No. 25) and a chimeric Fc- |
| | fragment of IgG subtype IgG2/4 subtype (SEQ ID No. 19) (CCN5(dIII)- |
| | HLn8-Fcv2.3). |
| 32 | Signal peptide from human serum albumin (MKWVTFISLLFLFSSAYS) |
| 33 | Fusion protein of SEQ ID No. 28 wherein the signal peptide from human |
| | serum albumin (SEQ ID No. 32) is appended N-terminally of the fusion protein. |
| 34 | DNA sequence encoding fusion protein of sequence No. 33 |
| 35 | DNA sequence encoding fusion protein of sequence No. 33 and further |
| | appended at the 5′ end by a KOZAK sequence, GCCACC, and at the 3′ end |
| | by a translation stop codon. |
| 36 | DNA sequence encoding fusion protein of sequence No. 35 and further |
| | including gateway AttB recombinase sites at both ends. |
| 37 | Amino acids 194-237 of human WISP2/CCN5 (domain III/TSP-1 |
| | homology domain) (44 aa fragment) |
| 38 | Amino acids 194-237 of human WISP2/CCN5 (domain III/TSP-1 |
| | homology domain) (44 aa fragment), where amino acid in position 195 |
| | (proline) is substituted with alanine |
| 39 | Linker (AEAAAKEAAAKEAAAKEAAAKEAAAKEAAAKEAAAKEAAAKAAA) |
| 40 | Fusion protein comprising amino acid 194-246 of CCN5 (SEQ ID. No. 1), |
| | fused C-terminal of a peptide linker (SEQ ID No. 39) and an Fc-fragment of |
| | IgG subtype IgG4 of SEQ ID NO. 15 (S228P, F234A, L235A, K447-, Eu- |
| | numbering) (Fc-HLn8-CCN5(dIII)). |
| 41 | Fusion protein comprising amino acid 194-246 of CCN5 (SEQ ID. No. 7), |
| | where amino acid in position 195 (proline) is substituted with alanine, fused |
| | C-terminal of a peptide linker (SEQ ID No. 39) and an Fc-fragment of IgG |
| | subtype IgG4 of SEQ ID NO. 15 (S228P, F234A, L235A, K447-, Eu- |
| | numbering) (Fc-HLn8-CCN5(dIII)-P195A). |
| 42 | Amino acids 229-272 of human Cyr61/CCN1 (domain III/TSP-1 |
| | homology domain) (44 aa fragment), where amino acid in position 230 |
| | (isoleucine) is substituted with alanine |
| 43 | Amino acids 199-242 of human CTGF/CCN2 (domain III/TSP-1 |
| | homology domain) (44 aa fragment), where amino acid in position 200 |
| | (leucine) is substituted with alanine |
| 44 | Amino acids 206-249 of human NOV/CCN3 (domain III/TSP-1 homology |
| | domain) (44 aa fragment), where amino acid in position 207 (isoleucine) is |
| | substituted with alanine |
| 45 | Amino acids 216-259 of human WISP1/CCN4 (domain III/TSP-1 |
| | homology domain) (44 aa fragment), where amino acid in position 217 |
| | (isoleucine) is substituted with alanine |
| 46 | Amino acids 209-252 of human WISP3/CCN6 (domain III/TSP-1 homology |
| | domain) (44 aa fragment), where amino acid in position 210 (leucine) is |
| | substituted with alanine |
| 47 | Amino acids 229-280 of human Cyr61/CCN1 (domain III/TSP-1 |
| | homology domain) (long fragment), where amino acid in position 230 |
| | (isoleucine) is substituted with alanine |
| 48 | Amino acids 199-250 of human CTGF/CCN2 (domain III/TSP-1 |
| | homology domain) (long fragment), where amino acid in position 200 |
| | (leucine) is substituted with alanine |
| 49 | Amino acids 206-258 of human NOV/CCN3 (domain III/TSP-1 homology |
| | domain) (long fragment), where amino acid in position 207 (isoleucine) is |
| | substituted with alanine |
| 50 | Amino acids 216-267 of human WISP1/CCN4 (domain III/TSP-1 homology |
| | domain) (long fragment), where amino acid in position 217 (isoleucine) is |
| | substituted with alanine |
| 51 | Amino acids 209-263 of human WISP3/CCN6 (domain III/TSP-1 |
| | homology domain) (long fragment), where amino acid in position 210 |
| | (leucine) is substituted with alanine |
| 52 | Amino acids 25-609 of human serum albumin |
| 53 | Amino acids 20-698 of of human serotransferrin |
| 54 | Fc-fragment of IgG1, disulfide bridge stabilized (R292C, V302C), |
| | aglycosylated (N297G) and with monomer-generating mutations (C220Q, |
| | C226Q, C229Q, T366R, L368H, P395K, K409T, M428L), Eu numbering) |
| 55 | Fc-fragment being a chimera of the hinge region of IgG2 and the constant |
| | heavy domains 2 and 3 of IgG4 with a deletion of the carboxyl-terminal |
| | K477- and with monomer-generating mutations (C219Q, C220Q, C226Q, |
| | C229Q, L351F, T366R, P395K, F405R, Y407E) and half-life extending |
| | mutations (M252Y, S254T, T256E) (Eu numbering). |
| 56 | Amino acids 194-250 of human WISP2/CCN5 (domain III/TSP-1 homology domain) |
| 57 | Linker (IEGRMD) |
| 58 | Fusion protein comprising amino acid 194-250 of CCN5 (SEQ ID. No. 56), |
| | fused N-terminal of a peptide linker (SEQ ID No. 57) and an Fc-fragment of |
| | IgG subtype IgG4 of SEQ ID NO. 15 (S228P, F234A, L235A, K447-, Eu-numbering) |
| 59 | Fusion protein comprising amino acid 194-250 of CCN5 (SEQ ID. No. 56), |
| | fused N-terminal of a peptide linker (SEQ ID No. 57) and an Fc-fragment of |
| | IgG subtype IgG4 of SEQ ID NO. 15 (S228P, F234A, L235A, K447-, Eu- |
| | numbering) that is appended amino-terminally by the signal peptide from |
| | human serum albumin (SEQ ID No. 32) |
| 60 | DNA sequence encoding fusion protein of SEQ ID No. 59 codon-optimized |
| | for expression in hamster cells and further appended at the 5′ end by a |
| | KOZAK sequence, GCCACC, and at the 3′ end by a translation stop codon. |
| 61 | DNA sequence of SEQ ID no 60 encoding fusion protein of SEQ ID No. 59 |
| | and further including gateway AttB recombinase sites at both ends. |
| 62 | Amino acids 194-249 of human WISP2/CCN5 (domain III/TSP-1 |
| | homology domain) (long fragment), where amino acid in position 245 |
| | (proline) is substituted with leucine. |
| 63 | Linker (GRMD) |
| 64 | Amino acids 194-249 of human WISP2/CCN5 (domain III/TSP-1 |
| | homology domain), where amino acid in position 245 (proline) is |
| | substituted with leucine (SEQ ID. No. 62), fused N-terminal of a peptide |
| | linker (SEQ ID No. 63) and an Fc-fragment of IgG subtype IgG4 of SEQ ID |
| | NO. 15 (S228P, F234A, L235A, K447-, Eu-numbering) that is appended |
| | amino-terminally by the signal peptide from human serum albumin (SEQ ID No. 32) |
| 65 | Linker (TEGRMD) |
| 66 | Amino acids 194-246 of human WISP2/CCN5 (domain III/TSP-1 |
| | homology domain) (SEQ ID No. 1), fused N-terminal of a peptide linker |
| | (SEQ ID No. 65) and an Fc-fragment of IgG subtype IgG4 of SEQ ID NO. |
| | 15 (S228P, F234A, L235A, K447-, Eu-numbering) that is appended amino- |
| | terminally by the signal peptide from human serum albumin (SEQ ID No. 32) |
| 67 | Linker (TAEAAAKA) |
| 68 | Amino acids 194-246 of human WISP2/CCN5 (domain III/TSP-1 |
| | homology domain) (SEQ ID No. 1), fused N-terminal of a peptide linker |
| | (SEQ ID No. 67) and an Fc-fragment of IgG subtype IgG4 of SEQ ID NO. |
| | 15 (S228P, F234A, L235A, K447-, Eu-numbering) that is appended amino- |
| | terminally by the signal peptide from human serum albumin (SEQ ID No. 32) |
| 69 | Amino acids 194-246 of human WISP2/CCN5 (domain III/TSP-1 |
| | homology domain) (SEQ ID No. 1), fused N-terminal of a peptide linker |
| | (SEQ ID No. 65) and a chimeric Fc fragment composed of the hinge region |
| | of IgG2 and the constant heavy domains 2 and 3 of IgG4 with a deletion of |
| | the carboxyl-terminal K477- (Eu numbering) of SEQ ID NO. 19 that is |
| | appended amino-terminally by the signal peptide from human serum |
| | albumin (SEQ ID No. 32) |
| 70 | Fusion protein of the SEQ ID No. 27 (comprising the amino acids 194-237 |
| | of domain III of CCN5, wherein proline position 195 is replaced by alanine |
| | (SEQ ID No. 38), coupled N-terminally of a linker of SEQ ID No. 21 that is |
| | further coupled N-terminal to the IgG2/4 Fc-fragment of SEQ ID NO. 19.) |
| | that is appended amino-terminally by the signal peptide from human serum |
| | albumin (SEQ ID No. 32) |
| 71 | DNA sequence encoding fusion protein of SEQ ID No. 70 codon-optimized |
| | for expression in hamster cells and further appended at the 5′ end by a |
| | KOZAK sequence, GCCACC, and at the 3′ end by a translation stop codon. |
| 72 | DNA sequence of SEQ ID no 71 encoding fusion protein of SEQ ID No. 70 |
| | and further including gateway AttB recombinase sites at both ends. |
| 73 | Amino acids 206-249 of human NOV/CCN3 (domain III/TSP-1 homology |
| | domain), where amino acid in position 207 (isoleucine) is substituted with |
| | alanine (SEQ ID No. 44), fused N-terminal of a peptide linker (SEQ ID No. |
| | 21) and a chimeric Fc fragment composed of the hinge region of IgG2 and |
| | the constant heavy domains 2 and 3 of IgG4 with a deletion of the carboxyl- |
| | terminal K477- (Eu numbering) of SEQ ID NO. 19 |
| 74 | Fusion protein (SEQ ID No. 73) comprising amino acids 206-249 of |
| | human NOV/CCN3 (domain III/TSP-1 homology domain), where amino |
| | acid in position 207 (isoleucine) is substituted with alanine (SEQ ID No. |
| | 44), fused N-terminal of a peptide linker (SEQ ID No. 21) and a chimeric Fc |
| | fragment composed of the hinge region of IgG2 and the constant heavy |
| | domains 2 and 3 of IgG4 with a deletion of the carboxyl-terminal K477- (Eu |
| | numbering) of SEQ ID NO. 19 that is appended amino-terminally by the |
| | signal peptide from human serum albumin (SEQ ID No. 32) |
| 75 | DNA sequence encoding fusion protein of SEQ ID No. 74 codon-optimized |
| | for expression in hamster cells and further appended at the 5′ end by a |
| | KOZAK sequence, GCCACC, and at the 3′ end by a translation stop codon. |
| 76 | DNA sequence of SEQ ID No 75 encoding fusion protein of SEQ ID No. 74 |
| | and further including gateway AttB recombinase sites at both ends. |
| 77 | Fusion protein (SEQ ID No 41) comprising amino acid 194-246 of CCN5 |
| | (SEQ ID. No. 7), where amino acid in position 195 (proline) is substituted |
| | with alanine, fused C-terminal of a peptide linker (SEQ ID No. 39) and an |
| | Fc-fragment of IgG subtype IgG4 of SEQ ID NO. 15 (S228P, F234A, |
| | L235A, K447-, Eu-numbering) (Fc-HLn8-CCN5(dIII)-P195A), that is |
| | appended amino-terminally by the signal peptide from human serum |
| | albumin (SEQ ID No. 32) |
| 78 | DNA sequence encoding fusion protein of SEQ ID No. 77 codon-optimized |
| | for expression in hamster cells and further appended at the 5′ end by a |
| | KOZAK sequence, GCCACC, and at the 3′ end by a translation stop codon. |
| 79 | DNA sequence of SEQ ID no 78 encoding fusion protein of SEQ ID No. 77 |
| | and further including gateway AttB recombinase sites at both ends. |
| 80 | Amino acids 206-249 of human NOV/CCN3 (domain III/TSP-1 homology |
| | domain), where amino acid in position 207 (isoleucine) is substituted with |
| | alanine (SEQ ID No. 44), fused N-terminal of a peptide linker (SEQ ID No. |
| | 22) and a chimeric Fc fragment composed of the hinge region of IgG2 and |
| | the constant heavy domains 2 and 3 of IgG4 with a deletion of the carboxyl- |
| | terminal K477- (Eu numbering) of SEQ ID NO. 19 |
| 81 | Fusion protein (SEQ ID No 81) comprising amino acids 206-249 of human |
| | NOV/CCN3 (domain III/TSP-1 homology domain), where amino acid in |
| | position 207 (isoleucine) is substituted with alanine (SEQ ID No. 44), fused |
| | N-terminal of a peptide linker (SEQ ID No. 22) and a chimeric Fc fragment |
| | composed of the hinge region of IgG2 and the constant heavy domains 2 and |
| | 3 of IgG4 with a deletion of the carboxyl-terminal K477- (Eu numbering) of |
| | SEQ ID NO. 19, that is appended amino-terminally by the signal peptide |
| | from human serum albumin (SEQ ID No. 32) |
| 82 | DNA sequence encoding fusion protein of SEQ ID No. 81 codon-optimized |
| | for expression in hamster cells and further appended at the 5′ end by a |
| | KOZAK sequence, GCCACC, and at the 3′ end by a translation stop codon. |
| 83 | DNA sequence of SEQ ID no 82 encoding fusion protein of SEQ ID No. 81 |
| | and further including gateway AttB recombinase sites at both ends. |
| 84 | Fusion protein comprising amino acids 206-249 of human NOV/CCN3 |
| | (domain III/TSP-1 homology domain), where amino acid in position 207 |
| | (isoleucine) is substituted with alanine (SEQ ID No. 44), fused N-terminal |
| | of a peptide linker (SEQ ID No. 21) and a Fc-fragment being a chimera of |
| | the hinge region of IgG2 and the constant heavy domains 2 and 3 of IgG4 |
| | with a deletion of the carboxyl-terminal K477- and with monomer- |
| | generating mutations (C219Q, C220Q, C226Q, C229Q, L351F, T366R, |
| | P395K, F405R, Y407E) and half-life extending mutations (M252Y, S254T, |
| | T256E) (Eu numbering) of SEQ ID NO. 55 |
| 85 | Fusion protein (SEQ ID No 84) comprising amino acids 206-249 of human |
| | NOV/CCN3 (domain III/TSP-1 homology domain), where amino acid in |
| | position 207 (isoleucine) is substituted with alanine (SEQ ID No. 44), fused |
| | N-terminal of a peptide linker (SEQ ID No. 21) and a Fc-fragment being a |
| | chimera of the hinge region of IgG2 and the constant heavy domains 2 and 3 |
| | of IgG4 with a deletion of the carboxyl-terminal K477- and with monomer- |
| | generating mutations (C219Q, C220Q, C226Q, C229Q, L351F, T366R, |
| | P395K, F405R, Y407E) and half-life extending mutations (M252Y, S254T, |
| | T256E) (Eu numbering) of SEQ ID NO. 55, that is appended amino- |
| | terminally by the signal peptide from human serum albumin (SEQ ID No. 32) |
| 86 | DNA sequence encoding fusion protein of SEQ ID No. 85 codon-optimized |
| | for expression in hamster cells and further appended at the 5′ end by a |
| | KOZAK sequence, GCCACC, and at the 3′ end by a translation stop codon. |
| 87 | DNA sequence of SEQ ID no 86 encoding fusion protein of SEQ ID No. 85 |
| | and further including gateway AttB recombinase sites at both ends. |
| 88 | Fusion protein comprising amino acids 206-249 of human NOV/CCN3 |
| | (domain III/TSP-1 homology domain), where amino acid in position 207 |
| | (isoleucine) is substituted with alanine (SEQ ID No. 44), fused N-terminal |
| | of a peptide linker (SEQ ID No. 21) and a Fc-fragment of IgG1, disulfide |
| | bridge stabilized (R292C, V302C), aglycosylated (N297G) and with |
| | monomer-generating mutations (C220Q, C226Q, C229Q, T366R, L368H, |
| | P395K, K409T, M428L), Eu numbering) of SEQ ID NO. 54 |
| 89 | Fusion protein (SEQ ID No. 88) comprising amino acids 206-249 of |
| | human NOV/CCN3 (domain III/TSP-1 homology domain), where amino |
| | acid in position 207 (isoleucine) is substituted with alanine (SEQ ID No. |
| | 44), fused N-terminal of a peptide linker (SEQ ID No. 21) and a Fc- |
| | fragment of IgG1, disulfide bridge stabilized (R292C, V302C), |
| | aglycosylated (N297G) and with monomer-generating mutations (C220Q, |
| | C226Q, C229Q, T366R, L368H, P395K, K409T, M428L), Eu numbering) of |
| | SEQ ID NO. 54, that is appended amino-terminally by the signal peptide |
| | from human serum albumin (SEQ ID No. 32) |
| 90 | DNA sequence encoding fusion protein of SEQ ID No. 89 codon-optimized |
| | for expression in hamster cells and further appended at the 5′ end by a |
| | KOZAK sequence, GCCACC, and at the 3′ end by a translation stop codon. |
| 91 | DNA sequence of SEQ ID no 90 encoding fusion protein of SEQ ID No. 89 |
| | and further including gateway AttB recombinase sites at both ends. |
| 92 | Multifunctional fusion tag comprised of 6xHis tag, HaloTag and Sumo* elements |
| 93 | GS-linker |
| 94 | Fusion protein comprising amino acids 206-249 of human NOV/CCN3 |
| | (domain III/TSP-1 homology domain), where amino acid in position 207 |
| | (isoleucine) is substituted with alanine (SEQ ID No. 44), fused C-terminal |
| | of a peptide linker (SEQ ID No. 93) and a multifunctional fusion tag |
| | comprised of 6xHis tag, HaloTag and Sumo* elements of SEQ ID NO. 92 |
| 95 | DNA sequence encoding fusion protein of SEQ ID No. 114 codon- |
| | optimized for expression in hamster cells and further appended at the 5′ end |
| | by a KOZAK sequence, GCCACC, and at the 3′ end by a translation stop codon. |
| 96 | DNA sequence of SEQ ID no 95 encoding fusion protein of SEQ ID No. |
| | 114 and further including gateway AttB recombinase sites at both ends. |
| 97 | Fusion protein of the invention comprising the amino acids 194-237 of |
| | CCN5, wherein proline position 195 is replaced by alanine (SEQ ID No. |
| | 38), coupled N-terminally of a linker of SEQ ID No. 21 that is further |
| | coupled N-terminal to amino acids 25-609 of human serum albumin (SEQ |
| | ID No. 52) |
| 98 | Fusion protein (SEQ ID No. 97) comprising the amino acids 194-237 of |
| | CCN5, wherein proline position 195 is replaced by alanine (SEQ ID No. |
| | 38), coupled N-terminally of a linker of SEQ ID No. 21 that is further |
| | coupled N-terminal to amino acids 25-609 of human serum albumin (SEQ |
| | ID No. 52), that is appended amino-terminally by the signal peptide from |
| | human serum albumin (SEQ ID No. 32) |
| 99 | DNA sequence encoding fusion protein of SEQ ID No. 98 codon-optimized |
| | for expression in hamster cells and further appended at the 5′ end by a |
| | KOZAK sequence, GCCACC, and at the 3′ end by a translation stop codon. |
| 100 | DNA sequence of SEQ ID no 99 encoding fusion protein of SEQ ID No. 98 |
| | and further including gateway AttB recombinase sites at both ends. |
| 101 | Amino acids 25-606 of human serum albumin |
| 102 | Fusion protein of the invention comprising amino acids 194-246 of human |
| | WISP2/CCN5, where amino acid in position 195 (proline) is substituted |
| | with alanine (SEQ ID No. 7), coupled C-terminally of a linker of SEQ ID |
| | No. 22 that is further coupled C-terminal to amino acids 25-606 of human |
| | serum albumin (SEQ ID No. 101) |
| 103 | Fusion protein (SEQ ID No. 102) comprising amino acids 194-246 of |
| | human WISP2/CCN5, where amino acid in position 195 (proline) is |
| | substituted with alanine (SEQ ID No. 7), coupled C-terminally of a linker of |
| | SEQ ID No. 22 that is further coupled C-terminal to amino acids 25-606 of |
| | human serum albumin (SEQ ID No. 101), that is appended amino-terminally |
| | by the signal peptide from human serum albumin (SEQ ID No. 32) |
| 104 | DNA sequence encoding fusion protein of SEQ ID No. 103 codon- |
| | optimized for expression in hamster cells and further appended at the 5′ end |
| | by a KOZAK sequence, GCCACC, and at the 3′ end by a translation stop codon. |
| 105 | DNA sequence of SEQ ID no 104 encoding fusion protein of SEQ ID No. |
| | 103 and further including gateway AttB recombinase sites at both ends. |
| 106 | Fusion protein of the invention comprising amino acids 206-249 of human |
| | NOV/CCN3 (domain III/TSP-1 homology domain), where amino acid in |
| | position 207 (isoleucine) is substituted with alanine (SEQ ID No. 44), |
| | coupled N-terminally of a linker of SEQ ID No. 21 that is further coupled |
| | N-terminal to amino acids 25-609 of human serum albumin (SEQ ID No. 52) |
| 107 | Fusion protein (SEQ ID No. 106) comprising amino acids 206-249 of |
| | human NOV/CCN3 (domain III/TSP-1 homology domain), where amino |
| | acid in position 207 (isoleucine) is substituted with alanine (SEQ ID No. |
| | 44), coupled N-terminally of a linker of SEQ ID No. 21 that is further |
| | coupled N-terminal to amino acids 25-609 of human serum albumin (SEQ |
| | ID No. 52), that is appended amino-terminally by the signal peptide from |
| | human serum albumin (SEQ ID No. 32) |
| 108 | DNA sequence encoding fusion protein of SEQ ID No. 107 codon- |
| | optimized for expression in hamster cells and further appended at the 5′ end |
| | by a KOZAK sequence, GCCACC, and at the 3′ end by a translation stop codon. |
| 109 | DNA sequence of SEQ ID no 108 encoding fusion protein of SEQ ID No. |
| | 108 and further including gateway AttB recombinase sites at both ends. |
| 110 | Fusion protein of the invention comprising amino acids 206-249 of human |
| | NOV/CCN3 (domain III/TSP-1 homology domain), where amino acid in |
| | position 207 (isoleucine) is substituted with alanine (SEQ ID No. 44), |
| | coupled N-terminally of a linker of SEQ ID No. 22 that is further coupled |
| | N-terminal to amino acids 25-609 of human serum albumin (SEQ ID No. |
| | 52) |
| 111 | Fusion protein (SEQ ID No. 110) comprising amino acids 206-249 of |
| | human NOV/CCN3 (domain III/TSP-1 homology domain), where amino |
| | acid in position 207 (isoleucine) is substituted with alanine (SEQ ID No. |
| | 44), coupled N-terminally of a linker of SEQ ID No. 22 that is further |
| | coupled N-terminal to amino acids 25-609 of human serum albumin (SEQ |
| | ID No. 52), that is appended amino-terminally by the signal peptide from |
| | human serum albumin (SEQ ID No. 32) |
| 112 | DNA sequence encoding fusion protein of SEQ ID No. 111 codon- |
| | optimized for expression in hamster cells and further appended at the 5′ end |
| | by a KOZAK sequence, GCCACC, and at the 3′ end by a translation stop codon. |
| 113 | DNA sequence of SEQ ID no 112 encoding fusion protein of SEQ ID No. |
| | 111 and further including gateway AttB recombinase sites at both ends. |
| 114 | Fusion protein (SEQ ID No. 94) comprising amino acids 206-249 of |
| | human NOV/CCN3 (domain III/TSP-1 homology domain), where amino |
| | acid in position 207 (isoleucine) is substituted with alanine (SEQ ID No. |
| | 44), fused C-terminal of a peptide linker (SEQ ID No. 93) and a |
| | multifunctional fusion tag comprised of 6xHis tag, HaloTag and Sumo* |
| | elements of SEQ ID NO. 92, that is appended amino-terminally by the |
| | signal peptide from human serum albumin (SEQ ID No. 32) |
| 115 | Potential section F of formula I |
| 116 | Potential section F of formula I |
| 117 | Potential section F of formula I |
| 118 | Potential section F of formula I |
| 119 | Potential section F of formula I |
| 120 | Potential section F of formula I |
| 121 | GGGGS linker |

Numbering of CCN proteins according to uniprot database, as described in the “Detailed description of the invention”, supra. Numbering of the Fc-fragments according to the Eu-numbering system as described in the “Detailed description of the invention”, supra.

1.-24. (canceled)

25. An isolated amino acid sequence having the amino acid sequence of SEQ ID NO: 20.


SEQUENCE LISTING (121 sequences)

SEQ ID NO: 1 - protein, 53 aa, Homo sapiens
CPEWSTAWGPCSTTCGLGMATRVSNQNRFCRLETQRRLCLSRPCPPSRGRSPQ

SEQ ID NO: 2 - protein, 44 aa, Homo sapiens
CIEQTTEWTACSKSCGMGFSTRVTNRNRQCEMLKQTRLCMVRPC

SEQ ID NO: 3 - protein, 44 aa, Homo sapiens
CLVQTTEWSACSKTCGMGISTRVTNDNASCRLEKQSRLCMVRPC

SEQ ID NO: 4 - protein, 44 aa, Homo sapiens
CIVQTTSWSQCSKTCGTGISTRVTNDNPECRLVKETRICEVRPC

SEQ ID NO: 5 - protein, 44 aa, Homo sapiens
CIAYTSPWSPCSTSCGLGVSTRISNVNAQCWPEQESRLCNLRPC

SEQ ID NO: 6 - protein, 44 aa, Homo sapiens
CLVQATKWTPCSRTCGMGISNRVTNENSNCEMRKEKRLCYIQPC

SEQ ID NO: 7 - protein, 53 aa, synthetic construct
  REGION 1..53: note = Modified CCN5 sequence
CAEWSTAWGPCSTTCGLGMATRVSNQNRFCRLETQRRLCLSRPCPPSRGRSPQ

SEQ ID NO: 8 - protein, 55 aa, Homo sapiens
CLVQATKWTPCSRTCGMGISNRVTNENSNCEMRKEKRLCYIQPCDSNILKTIKIP

SEQ ID NO: 9 - protein, 53 aa, Homo sapiens
CIEQTTEWTACSKSCGMGFSTRVTNRNRQCEMLKQTRLCMVRPCEQEPEQPTD

SEQ ID NO: 10 - protein, 52 aa, Homo sapiens
CLVQTTEWSACSKTCGMGISTRVTNDNASCRLEKQSRLCMVRPCEADLEENI

SEQ ID NO: 11 - protein, 52 aa, Homo sapiens
CIVQTTSWSQCSKTCGTGISTRVTNDNPECRLVKETRICEVRPCGQPVYSSL

SEQ ID NO: 12 - protein, 52 aa, Homo sapiens
CIAYTSPWSPCSTSCGLGVSTRISNVNAQCWPEQESRLCNLRPCDVDIHTLI

SEQ ID NO: 13 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - protein, 228 aa, Homo sapiens
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 15 - protein, 228 aa, synthetic construct
  REGION 1..228: note = Modified Fc fragment
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 16 - protein, 232 aa, synthetic construct
  REGION 1..232: note = Modified Fc fragment
EPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 231 aa, synthetic construct
  REGION 1..231: note = Modified Fc fragment
EPKSCDKTHTCPPCPAPPVAGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 227 aa, synthetic construct
  REGION 1..227: note = Modified Fc fragment
ESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 19 - protein, 227 aa, synthetic construct
  REGION 1..227: note = Modified Fc fragment
ERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 20 - protein, 6 aa, synthetic construct
  REGION 1..6: note = Linker
TEGRMD

SEQ ID NO: 21 - protein, 5 aa, synthetic construct
  REGION 1..5: note = Linker
EAAAK

SEQ ID NO: 22 - protein, 40 aa, synthetic construct
  REGION 1..40: note = Linker
EAAAKEAAAKEAAAKEAAAKEAAAKEAAAKEAAAKEAAAK

SEQ ID NO: 23 - protein, 42 aa, synthetic construct
  REGION 1..42: note = Linker
TAEAAAKEAAAKEAAAKEAAAKEAAAKEAAAKEAAAKEAAAK

SEQ ID NO: 24 - protein, 43 aa, synthetic construct
  REGION 1..43: note = Linker
EAAAKEAAAKEAAAKEAAAKEAAAKEAAAKEAAAKEAAAKAAA

SEQ ID NO: 25 - protein, 45 aa, synthetic construct
  REGION 1..45: note = Linker
TAEAAAKEAAAKEAAAKEAAAKEAAAKEAAAKEAAAKEAAAKAAA

SEQ ID NO: 26 - protein, 276 aa, synthetic construct
  REGION 1..276: note = Fusion protein
CPEWSTAWGPCSTTCGLGMATRVSNQNRFCRLETQRRLCLSRPCEAAAKERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 27 - protein, 276 aa, synthetic construct
  REGION 1..276: note = Fusion protein
CAEWSTAWGPCSTTCGLGMATRVSNQNRFCRLETQRRLCLSRPCEAAAKERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 28 - protein, 287 aa, synthetic construct
  REGION 1..287: note = Fusion protein
CPEWSTAWGPCSTTCGLGMATRVSNQNRFCRLETQRRLCLSRPCPPSRGRSPQTEGRMDESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 29 - protein, 286 aa, synthetic construct
  REGION 1..286: note = Fusion protein
CPEWSTAWGPCSTTCGLGMATRVSNQNRFCRLETQRRLCLSRPCPPSRGRSPQTEGRMDESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 30 - protein, 286 aa, synthetic construct
  REGION 1..286: note = Fusion protein
CPEWSTAWGPCSTTCGLGMATRVSNQNRFCRLETQRRLCLSRPCPPSRGRSPQTEGRMDERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 31 - protein, 325 aa, synthetic construct
  REGION 1..325: note = Fusion protein
CPEWSTAWGPCSTTCGLGMATRVSNQNRFCRLETQRRLCLSRPCPPSRGRSPQTAEAAAKEAAAKEAAAKEAAAKEAAAKEAAAKEAAAKEAAAKAAAERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 32 - protein, 18 aa, Homo sapiens
MKWVTFISLLFLFSSAYS

SEQ ID NO: 33 - protein, 305 aa, synthetic construct
  REGION 1..305: note = Fusion protein
MKWVTFISLLFLFSSAYSCPEWSTAWGPCSTTCGLGMATRVSNQNRFCRLETQRRLCLSRPCPPSRGRSPQTEGRMDESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 34 - DNA, 915 bp, synthetic construct
  misc_feature 1..915: note = Fusion protein
  source 1..915: mol_type = other DNA
atgaaatgggtcacctttatctccctgctgttcctgttctcctccgcctactcttgccctgagtggtctacagcttggggcccttgctctaccacctgtggactcggcatggccaccagagtgtctaaccagaacagattctgccggctggaaacccagcggagactgtgcctgtctagaccctgtcctcctagcagaggcagatcccctcagaccgagggcagaatggacgagtctaagtacggccctccttgtcctccatgtcctgctccagaagctgctggcggcccttccgtgtttctgttccctccaaagcctaaggacaccctgatgatctctcggacccctgaagtgacctgcgtggtggtggatgtgtcccaagaggatcccgaggtgcagttcaattggtacgtggacggcgtggaagtgcacaacgccaagaccaagcctagagaggaacagttcaactccacctacagagtggtgtccgtgctgaccgtgctgcaccaggattggctgaacggcaaagagtacaagtgcaaggtgtccaacaagggcctgccttccagcatcgaaaagaccatctccaaggccaagggccagcctagggaaccccaggtttacaccctgcctccaagccaagaggaaatgaccaagaaccaggtgtccctgacctgcctggtcaagggcttctacccttccgatatcgccgtggaatgggagagcaatggccagcctgagaacaactacaagaccacacctcctgtgctggactccgacggctccttctttctgtactcccgcctgaccgtggacaagtccagatggcaagagggcaacgtgttctcctgctccgtgatgcacgaggccctgcacaatcactacacccagaagtccctgtctctgtccctgggc

SEQ ID NO: 35 - DNA, 924 bp, synthetic construct
  misc_feature 1..924: note = Fusion protein
  source 1..924: mol_type = other DNA
gccaccatgaaatgggtcacctttatctccctgctgttcctgttctcctccgcctactcttgccctgagtggtctacagcttggggcccttgctctaccacctgtggactcggcatggccaccagagtgtctaaccagaacagattctgccggctggaaacccagcggagactgtgcctgtctagaccctgtcctcctagcagaggcagatcccctcagaccgagggcagaatggacgagtctaagtacggccctccttgtcctccatgtcctgctccagaagctgctggcggcccttccgtgtttctgttccctccaaagcctaaggacaccctgatgatctctcggacccctgaagtgacctgcgtggtggtggatgtgtcccaagaggatcccgaggtgcagttcaattggtacgtggacggcgtggaagtgcacaacgccaagaccaagcctagagaggaacagttcaactccacctacagagtggtgtccgtgctgaccgtgctgcaccaggattggctgaacggcaaagagtacaagtgcaaggtgtccaacaagggcctgccttccagcatcgaaaagaccatctccaaggccaagggccagcctagggaaccccaggtttacaccctgcctccaagccaagaggaaatgaccaagaaccaggtgtccctgacctgcctggtcaagggcttctacccttccgatatcgccgtggaatgggagagcaatggccagcctgagaacaactacaagaccacacctcctgtgctggactccgacggctccttctttctgtactcccgcctgaccgtggacaagtccagatggcaagagggcaacgtgttctcctgctccgtgatgcacgaggccctgcacaatcactacacccagaagtccctgtctctgtccctgggctaa

SEQ ID NO: 36 - DNA, 997 bp, synthetic construct
  misc_feature 1..997: note = Fusion protein
  source 1..997: mol_type = other DNA
ggggacaagtttgtacaaaaaagcaggctatggtaccgccaccatgaaatgggtcacctttatctccctgctgttcctgttctcctccgcctactcttgccctgagtggtctacagcttggggcccttgctctaccacctgtggactcggcatggccaccagagtgtctaaccagaacagattctgccggctggaaacccagcggagactgtgcctgtctagaccctgtcctcctagcagaggcagatcccctcagaccgagggcagaatggacgagtctaagtacggccctccttgtcctccatgtcctgctccagaagctgctggcggcccttccgtgtttctgttccctccaaagcctaaggacaccctgatgatctctcggacccctgaagtgacctgcgtggtggtggatgtgtcccaagaggatcccgaggtgcagttcaattggtacgtggacggcgtggaagtgcacaacgccaagaccaagcctagagaggaacagttcaactccacctacagagtggtgtccgtgctgaccgtgctgcaccaggattggctgaacggcaaagagtacaagtgcaaggtgtccaacaagggcctgccttccagcatcgaaaagaccatctccaaggccaagggccagcctagggaaccccaggtttacaccctgcctccaagccaagaggaaatgaccaagaaccaggtgtccctgacctgcctggtcaagggcttctacccttccgatatcgccgtggaatgggagagcaatggccagcctgagaacaactacaagaccacacctcctgtgctggactccgacggctccttctttctgtactcccgcctgaccgtggacaagtccagatggcaagagggcaacgtgttctcctgctccgtgatgcacgaggccctgcacaatcactacacccagaagtccctgtctctgtccctgggctaatctagaaacccagctttcttgtacaaagtggtcccc

SEQ ID NO: 37 - protein, 44 aa, Homo sapiens
CPEWSTAWGPCSTTCGLGMATRVSNQNRFCRLETQRRLCLSRPC

SEQ ID NO: 38 - protein, 44 aa, synthetic construct
  REGION 1..44: note = Modified CCN5 sequence
CAEWSTAWGPCSTTCGLGMATRVSNQNRFCRLETQRRLCLSRPC

SEQ ID NO: 39 - protein, 44 aa, synthetic construct
  REGION 1..44: note = Linker
AEAAAKEAAAKEAAAKEAAAKEAAAKEAAAKEAAAKEAAAKAAA

SEQ ID NO: 40 - protein, 325 aa, synthetic construct
  REGION 1..325: note = Fusion protein
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAEAAAKEAAAKEAAAKEAAAKEAAAKEAAAKEAAAKEAAAKAAACPEWSTAWGPCSTTCGLGMATRVSNQNRFCRLETQRRLCLSRPCPPSRGRSPQ

SEQ ID NO: 41 - protein, 325 aa, synthetic construct
  REGION 1..325: note = Fusion protein
ESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAEAAAKEAAAKEAAAKEAAAKEAAAKEAAAKEAAAKEAAAKAAACAEWSTAWGPCSTTCGLGMATRVSNQNRFCRLETQRRLCLSRPCPPSRGRSPQ

SEQ ID NO: 42 - protein, 44 aa, synthetic construct
  REGION 1..44: note = Modified CCN1 sequence
CAVQTTSWSQCSKTCGTGISTRVTNDNPECRLVKETRICEVRPC

SEQ ID NO: 43 - protein, 44 aa, synthetic construct
  REGION 1..44: note = Modified CCN2 sequence
CAVQTTEWSACSKTCGMGISTRVTNDNASCRLEKQSRLCMVRPC

SEQ ID NO: 44 - protein, 44 aa, synthetic construct
  REGION 1..44: note = Modified CCN3 sequence
CAEQTTEWTACSKSCGMGFSTRVTNRNRQCEMLKQTRLCMVRPC

SEQ ID NO: 45 - protein, 44 aa, synthetic construct
  REGION 1..44: note = Modified CCN4 sequence
CAAYTSPWSPCSTSCGLGVSTRISNVNAQCWPEQESRLCNLRPC

SEQ ID NO: 46 - protein, 44 aa, synthetic construct
  REGION 1..44: note = Modified CCN6 sequence
CAVQATKWTPCSRTCGMGISNRVTNENSNCEMRKEKRLCYIQPC

SEQ ID NO: 47 - protein, 52 aa, synthetic construct
  REGION 1..52: note = Modified CCN1 sequence
CAVQTTSWSQCSKTCGTGISTRVTNDNPECRLVKETRICEVRPCGQPVYSSL

SEQ ID NO: 48 - protein, 52 aa, synthetic construct
  REGION 1..52: note = Modified CCN2 sequence
CAVQTTEWSACSKTCGMGISTRVTNDNASCRLEKQSRLCMVRPCEADLEENI

SEQ ID NO: 49 - protein, 53 aa, synthetic construct
  REGION 1..53: note = Modified CCN3 sequence
CAEQTTEWTACSKSCGMGFSTRVTNRNRQCEMLKQTRLCMVRPCEQEPEQPTD

SEQ ID NO: 50 - protein, 52 aa, synthetic construct
  REGION 1..52: note = Modified CCN4 sequence
CAAYTSPWSPCSTSCGLGVSTRISNVNAQCWPEQESRLCNLRPCDVDIHTLI

SEQ ID NO: 51 - protein, 55 aa, synthetic construct
  REGION 1..55: note = Modified CCN6 sequence
CAVQATKWTPCSRTCGMGISNRVTNENSNCEMRKEKRLCYIQPCDSNILKTIKIP

SEQ ID NO: 52 - protein, 585 aa, Homo sapiens [sequence omitted]

SEQ ID NO: 53 - protein, 679 aa, Homo sapiens
VPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPEAGYFAIAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

SEQ ID NO: 54 - protein, 232 aa, synthetic construct
  REGION 1..232: note = Modified Fc fragment
EPKSQDKTHTQPPQPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSRDELTKNQVSLRCHVKGFYPSDIAVEWESNGQPENNYKTTKPVLDSDGSFFLYSTLTVDKSRWQQGNVFSCSVLHEALHNHYTQKSLSLSPGK

SEQ ID NO: 55 - protein, 227 aa, synthetic construct
  REGION 1..227: note = Modified Fc fragment
ERKQQVEQPPQPAPPVAGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTFPPSQEEMTKNQVSLRCLVKGFYPSDIAVEWESNGQPENNYKTTKPVLDSDGSFRLESRLTVDKSRWQEGNVFSCSVMHEALHNHYTQKSLSLSLG

SEQ ID NO: 56 - protein, 57 aa, Homo sapiens
CPEWSTAWGPCSTTCGLGMATRVSNQNRFCRLETQRRLCLSRPCPPSRGRSPQNSAF

SEQ ID NO: 57 - protein, 6 aa, synthetic construct
  REGION 1..6: note = Linker
IEGRMD

SEQ ID NO: 58 - protein, 291 aa, synthetic construct
  REGION 1..291: note = Fusion protein
CPEWSTAWGPCSTTCGLGMATRVSNQNRFCRLETQRRLCLSRPCPPSRGRSPQNSAFIEGRMDESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 59 - protein, 309 aa, synthetic construct
  REGION 1..309: note = Fusion protein
MKWVTFISLLFLFSSAYSCPEWSTAWGPCSTTCGLGMATRVSNQNRFCRLETQRRLCLSRPCPPSRGRSPQNSAFIEGRMDESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 60 - DNA, 936 bp, synthetic construct
  misc_feature 1..936: note = Fusion protein
  source 1..936: mol_type = other DNA
gccaccatgaaatgggtcacctttatctccctgctgttcctgttctcctccgcctactcttgccctgagtggtctacagcttggggcccttgctctaccacctgtggactcggcatggccaccagagtgtctaaccagaacagattctgccggctggaaacccagcggagactgtgtctgtccagaccttgtcctcctagccggggcagatcccctcagaactctgcctttatcgagggcagaatggacgagtctaagtacggccctccttgtccaccatgtcctgctccagaagctgctggcggcccttccgtgtttctgttccctccaaagcctaaggacaccctgatgatctctcggacccctgaagtgacctgcgtggtggtggatgtgtcccaagaggatcccgaggtgcagttcaattggtacgtggacggcgtggaagtgcacaacgccaagaccaagcctagagaggaacagttcaactccacctacagagtggtgtccgtgctgaccgtgctgcaccaggattggctgaacggcaaagagtacaagtgcaaggtgtccaacaagggcctgccttccagcatcgaaaagaccatctccaaggccaagggccagcctagggaaccccaggtttacaccctgcctccaagccaagaggaaatgaccaagaaccaggtgtccctgacctgcctggtcaagggcttctacccttccgatatcgccgtggaatgggagagcaatggccagcctgagaacaactacaagaccacacctcctgtgctggactccgacggctccttctttctgtactcccgcctgaccgtggacaagtccagatggcaagagggcaacgtgttctcctgctccgtgatgcacgaggccctgcacaatcactacacccagaagtccctgtctctgtccctgggctaa

SEQ ID NO: 61 - DNA, 1009 bp, synthetic construct
  misc_feature 1..1009: note = Fusion protein
  source 1..1009: mol_type = other DNA
ggggacaagtttgtacaaaaaagcaggctatggtaccgccaccatgaaatgggtcacctttatctccctgctgttcctgttctcctccgcctactcttgccctgagtggtctacagcttggggcccttgctctaccacctgtggactcggcatggccaccagagtgtctaaccagaacagattctgccggctggaaacccagcggagactgtgtctgtccagaccttgtcctcctagccggggcagatcccctcagaactctgcctttatcgagggcagaatggacgagtctaagtacggccctccttgtccaccatgtcctgctccagaagctgctggcggcccttccgtgtttctgttccctccaaagcctaaggacaccctgatgatctctcggacccctgaagtgacctgcgtggtggtggatgtgtcccaagaggatcccgaggtgcagttcaattggtacgtggacggcgtggaagtgcacaacgccaagaccaagcctagagaggaacagttcaactccacctacagagtggtgtccgtgctgaccgtgctgcaccaggattggctgaacggcaaagagtacaagtgcaaggtgtccaacaagggcctgccttccagcatcgaaaagaccatctccaaggccaagggccagcctagggaaccccaggtttacaccctgcctccaagccaagaggaaatgaccaagaaccaggtgtccctgacctgcctggtcaagggcttctacccttccgatatcgccgtggaatgggagagcaatggccagcctgagaacaactacaagaccacacctcctgtgctggactccgacggctccttctttctgtactcccgcctgaccgtggacaagtccagatggcaagagggcaacgtgttctcctgctccgtgatgcacgaggccctgcacaatcactacacccagaagtccctgtctctgtccctgggctaatctagaaacccagctttcttgtacaaagtggtcccc

SEQ ID NO: 62 - protein, 56 aa, synthetic construct
  REGION 1..56: note = Modified CCN5 sequence
CPEWSTAWGPCSTTCGLGMATRVSNQNRFCRLETQRRLCLSRPCPPSRGRSLQNSA

SEQ ID NO: 63 - protein, 4 aa, synthetic construct
  REGION 1..4: note = Linker
GRMD

SEQ ID NO: 64 - protein, 306 aa, synthetic construct
  REGION 1..306: note = Fusion protein
MKWVTFISLLFLFSSAYSCPEWSTAWGPCSTTCGLGMATRVSNQNRFCRLETQRRLCLSRPCPPSRGRSLQNSAGRMDESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 65 - protein, 6 aa, synthetic construct
  REGION 1..6: note = Linker
TEGRMD

SEQ ID NO: 66 - protein, 305 aa, synthetic construct; identical to SEQ ID NO: 33
  REGION 1..305: note = Fusion protein

SEQ ID NO: 67 - protein, 8 aa, synthetic construct
  REGION 1..8: note = Linker
TAEAAAKA

SEQ ID NO: 68 - protein, 307 aa, synthetic construct
  REGION 1..307: note = Fusion protein
MKWVTFISLLFLFSSAYSCPEWSTAWGPCSTTCGLGMATRVSNQNRFCRLETQRRLCLSRPCPPSRGRSPQTAEAAAKAESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 69 - protein, 304 aa, synthetic construct
  REGION 1..304: note = Fusion protein
MKWVTFISLLFLFSSAYSCPEWSTAWGPCSTTCGLGMATRVSNQNRFCRLETQRRLCLSRPCPPSRGRSPQTEGRMDERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 70 - protein, 294 aa, synthetic construct
  REGION 1..294: note = Fusion protein
MKWVTFISLLFLFSSAYSCAEWSTAWGPCSTTCGLGMATRVSNQNRFCRLETQRRLCLSRPCEAAAKERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 71 - DNA, 891 bp, synthetic construct
  misc_feature 1..891: note = Fusion protein
  source 1..891: mol_type = other DNA
gccaccatgaaatgggtcacctttatctccctgctgttcctgttctcctccgcctactcttgcgccgagtggtctacagcttggggcccttgttctaccacctgtggcctcggcatggccaccagagtgtccaaccagaacagattctgccggctggaaacccagcggagactgtgtttgtccagaccttgcgaggccgctgccaaagaaagaaagtgctgcgtggaatgccctccttgtcctgctcctcctgtggctggcccttccgtgtttctgttccctccaaagcctaaggacaccctgatgatctctcggacccctgaagtgacctgcgtggtggtggatgtgtcccaagaggatcccgaggtgcagttcaattggtacgtggacggcgtggaagtgcacaacgccaagaccaagcctagagaggaacagttcaactccacctacagagtggtgtccgtgctgaccgtgctgcaccaggattggctgaacggcaaagagtacaagtgcaaggtgtccaacaagggactgccctccagcatcgaaaagaccatctccaaggccaagggacagcccagagaaccccaggtgtacacactgcctccaagccaagaggaaatgaccaagaaccaggtgtccctgacctgcctggtcaagggcttctacccttccgatatcgccgtggaatgggagtccaatggccagcctgagaacaactacaagaccacacctccagtgctggactccgacggctccttctttctgtactcccgcctgaccgtggacaagtccagatggcaagagggcaacgtgttctcctgctccgtgatgcacgaggccctgcacaatcactacacccagaagtccctgtctctgtccctgggctaa

SEQ ID NO: 72 - DNA, 964 bp, synthetic construct
  misc_feature 1..964: note = Fusion protein
  source 1..964: mol_type = other DNA
ggggacaagtttgtacaaaaaagcaggctatggtaccgccaccatgaaatgggtcacctttatctccctgctgttcctgttctcctccgcctactcttgcgccgagtggtctacagcttggggcccttgttctaccacctgtggcctcggcatggccaccagagtgtccaaccagaacagattctgccggctggaaacccagcggagactgtgtttgtccagaccttgcgaggccgctgccaaagaaagaaagtgctgcgtggaatgccctccttgtcctgctcctcctgtggctggcccttccgtgtttctgttccctccaaagcctaaggacaccctgatgatctctcggacccctgaagtgacctgcgtggtggtggatgtgtcccaagaggatcccgaggtgcagttcaattggtacgtggacggcgtggaagtgcacaacgccaagaccaagcctagagaggaacagttcaactccacctacagagtggtgtccgtgctgaccgtgctgcaccaggattggctgaacggcaaagagtacaagtgcaaggtgtccaacaagggactgccctccagcatcgaaaagaccatctccaaggccaagggacagcccagagaaccccaggtgtacacactgcctccaagccaagaggaaatgaccaagaaccaggtgtccctgacctgcctggtcaagggcttctacccttccgatatcgccgtggaatgggagtccaatggccagcctgagaacaactacaagaccacacctccagtgctggactccgacggctccttctttctgtactcccgcctgaccgtggacaagtccagatggcaagagggcaacgtgttctcctgctccgtgatgcacgaggccctgcacaatcactacacccagaagtccctgtctctgtccctgggctaatctagaaacccagctttcttgtacaaagtggtcccc

SEQ ID NO: 73 - protein, 276 aa, synthetic construct
  REGION 1..276: note = Fusion protein
CAEQTTEWTACSKSCGMGFSTRVTNRNRQCEMLKQTRLCMVRPCEAAAKERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 74 - protein, 294 aa, synthetic construct
  REGION 1..294: note = Fusion protein
MKWVTFISLLFLFSSAYSCAEQTTEWTACSKSCGMGFSTRVTNRNRQCEMLKQTRLCMVRPCEAAAKERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 75 - DNA, 891 bp, synthetic construct
  misc_feature 1..891: note = Fusion protein
  source 1..891: mol_type = other DNA
gccaccatgaaatgggtcacctttatctccctgctgttcctgttctcctccgcctactcttgtgccgagcagaccacagagtggaccgcctgctctaagtcttgcggcatgggcttctccaccagagtgaccaaccggaacagacagtgcgagatgctgaagcagacccggctgtgtatggttcgaccttgcgaggccgctgccaaagaaagaaagtgctgcgtggaatgccctccttgtcctgctcctcctgtggctggcccttccgtgtttctgttccctccaaagcctaaggacaccctgatgatctctcggacccctgaagtgacctgcgtggtggtggatgtgtcccaagaggatcccgaggtgcagttcaattggtacgtggacggcgtggaagtgcacaacgccaagaccaagcctagagaggaacagttcaactccacctacagagtggtgtccgtgctgaccgtgctgcaccaggattggctgaacggcaaagagtacaagtgcaaggtgtccaacaagggcctgccttccagcatcgaaaagaccatctccaaggccaagggacagcccagagaaccccaggtgtacacactgcctccaagccaagaggaaatgaccaagaaccaggtgtccctgacctgcctggtcaagggcttctacccttccgatatcgccgtggaatgggagtccaatggccagcctgagaacaactacaagaccacacctccagtgctggactccgacggctccttctttctgtactcccgcctgaccgtggacaagtccagatggcaagagggcaacgtgttctcctgctccgtgatgcacgaggccctgcacaatcactacacccagaagtccctgtctctgtccctgggctaa

SEQ ID NO: 76 - DNA, 964 bp, synthetic construct
  misc_feature 1..964: note = Fusion protein
  source 1..964: mol_type = other DNA
ggggacaagtttgtacaaaaaagcaggctatggtaccgccaccatgaaatgggtcacctttatctccctgctgttcctgttctcctccgcctactcttgtgccgagcagaccacagagtggaccgcctgctctaagtcttgcggcatgggcttctccaccagagtgaccaaccggaacagacagtgcgagatgctgaagcagacccggctgtgtatggttcgaccttgcgaggccgctgccaaagaaagaaagtgctgcgtggaatgccctccttgtcctgctcctcctgtggctggcccttccgtgtttctgttccctccaaagcctaaggacaccctgatgatctctcggacccctgaagtgacctgcgtggtggtggatgtgtcccaagaggatcccgaggtgcagttcaattggtacgtggacggcgtggaagtgcacaacgccaagaccaagcctagagaggaacagttcaactccacctacagagtggtgtccgtgctgaccgtgctgcaccaggattggctgaacggcaaagagtacaagtgcaaggtgtccaacaagggcctgccttccagcatcgaaaagaccatctccaaggccaagggacagcccagagaaccccaggtgtacacactgcctccaagccaagaggaaatgaccaagaaccaggtgtccctgacctgcctggtcaagggcttctacccttccgatatcgccgtggaatgggagtccaatggccagcctgagaacaactacaagaccacacctccagtgctggactccgacggctccttctttctgtactcccgcctgaccgtggacaagtccagatggcaagagggcaacgtgttctcctgctccgtgatgcacgaggccctgcacaatcactacacccagaagtccctgtctctgtccctgggctaatctagaaacccagctttcttgtacaaagtggtcccc

SEQ ID NO: 77 - protein, 343 aa, synthetic construct
  REGION 1..343: note = Fusion protein
MKWVTFISLLFLFSSAYSESKYGPPCPPCPAPEAAGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGAEAAAKEAAAKEAAAKEAAAKEAAAKEAAAKEAAAKEAAAKAAACAEWSTAWGPCSTTCGLGMATRVSNQNRFCRLETQRRLCLSRPCPPSRGRSPQ

SEQ ID NO: 78 - DNA, 1038 bp, synthetic construct
  misc_feature 1..1038: note = Fusion protein
  source 1..1038: mol_type = other DNA
gccaccatgaaatgggtcacctttatctccctgctgttcctgttctccagcgcctactccgagtctaagtacggccctccttgtcctccatgtcctgctccagaagctgctggcggcccttccgtgtttctgttccctccaaagcctaaggacaccctgatgatctctcggacccctgaagtgacctgcgtggtggtggatgtgtcccaagaggatcccgaggtgcagttcaattggtacgtggacggcgtggaagtgcacaacgccaagaccaagcctagagaggaacagttcaactccacctacagagtggtgtccgtgctgaccgtgctgcaccaggattggctgaacggcaaagagtacaagtgcaaggtgtccaacaagggcctgccttccagcatcgaaaagaccatctccaaggccaagggccagcctagggaaccccaggtttacaccctgcctccaagccaagaggaaatgaccaagaaccaggtgtccctgacctgcctggtcaagggcttctacccttccgatatcgccgtggaatgggagagcaatggccagcctgagaacaactacaagaccacacctcctgtgctggactccgacggctccttctttctgtactcccgcctgaccgtggacaagtccagatggcaagagggcaacgtgttctcctgctccgtgatgcacgaggccctgcacaatcactacacccagaagtccctgtctctgtccctgggagctgaggccgctgctaaagaagctgccgctaaagaggccgcagccaaagaggcagccgccaaagaagccgctgcaaaagaggctgctgcaaaagaagcagcagctaaagaagctgctgccaaggccgctgcttgtgccgaatggtctacagcttggggcccttgctctaccacctgtggactcggcatggccaccagagtgtctaaccagaacagattctgccggctggaaacccagcggagactgtgcctgtctagaccctgtcctcctagcagaggcagatcccctcagtga

SEQ ID NO: 79 - DNA, 1111 bp, synthetic construct
  misc_feature 1..1111: note = Fusion protein
  source 1..1111: mol_type = other DNA
ggggacaagtttgtacaaaaaagcaggctatggtaccgccaccatgaaatgggtcacctttatctccctgctgttcctgttctccagcgcctactccgagtctaagtacggccctccttgtcctccatgtcctgctccagaagctgctggcggcccttccgtgtttctgttccctccaaagcctaaggacaccctgatgatctctcggacccctgaagtgacctgcgtggtggtggatgtgtcccaagaggatcccgaggtgcagttcaattggtacgtggacggcgtggaagtgcacaacgccaagaccaagcctagagaggaacagttcaactccacctacagagtggtgtccgtgctgaccgtgctgcaccaggattggctgaacggcaaagagtacaagtgcaaggtgtccaacaagggcctgccttccagcatcgaaaagaccatctccaaggccaagggccagcctagggaaccccaggtttacaccctgcctccaagccaagaggaaatgaccaagaaccaggtgtccctgacctgcctggtcaagggcttctacccttccgatatcgccgtggaatgggagagcaatggccagcctgagaacaactacaagaccacacctcctgtgctggactccgacggctccttctttctgtactcccgcctgaccgtggacaagtccagatggcaagagggcaacgtgttctcctgctccgtgatgcacgaggccctgcacaatcactacacccagaagtccctgtctctgtccctgggagctgaggccgctgctaaagaagctgccgctaaagaggccgcagccaaagaggcagccgccaaagaagccgctgcaaaagaggctgctgcaaaagaagcagcagctaaagaagctgctgccaaggccgctgcttgtgccgaatggtctacagcttggggcccttgctctaccacctgtggactcggcatggccaccagagtgtctaaccagaacagattctgccggctggaaacccagcggagactgtgcctgtctagaccctgtcctcctagcagaggcagatcccctcagtgatctagaaacccagctttcttgtacaaagtggtcccc

SEQ ID NO: 80 - protein, 311 aa, synthetic construct
  REGION 1..311: note = Fusion protein
CAEQTTEWTACSKSCGMGFSTRVTNRNRQCEMLKQTRLCMVRPCEAAAKEAAAKEAAAKEAAAKEAAAKEAAAKEAAAKEAAAKERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 81 - protein, 329 aa, synthetic construct
  REGION 1..329: note = Fusion protein
MKWVTFISLLFLFSSAYSCAEQTTEWTACSKSCGMGFSTRVTNRNRQCEMLKQTRLCMVRPCEAAAKEAAAKEAAAKEAAAKEAAAKEAAAKEAAAKEAAAKERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 82 - DNA, 996 bp, synthetic construct
  misc_feature 1..996: note = Fusion protein
  source 1..996: mol_type = other DNA
gccaccatgaaatgggtcacctttatctccctgctgttcctgttctcctccgcctactcttgtgccgagcagaccacagagtggaccgcctgctctaagtcttgcggcatgggcttctccaccagagtgaccaaccggaacagacagtgcgagatgctgaagcagacccggctgtgtatggttcgaccttgcgaggccgctgccaaagaggctgctgctaaagaagccgccgcaaaagaggcagcagcaaaagaggctgccgccaaagaggccgcagccaaagaagcagcagctaaagaggccgctgcaaaagaacggaagtgctgcgtggaatgccctccttgtcctgctcctcctgtggctggcccttccgtgtttctgttccctccaaagcctaaggacaccctgatgatctctcggacccctgaagtgacctgcgtggtggtggatgtgtcccaagaggatcccgaggtgcagttcaattggtacgtggacggcgtggaagtgcacaacgccaagaccaagcctagagaggaacagttcaactccacctacagagtggtgtccgtgctgaccgtgctgcaccaggattggctgaacggcaaagagtacaagtgcaaggtgtccaacaagggcctgccttccagcatcgaaaagaccatctccaaggccaagggacagcccagagaaccccaggtgtacacactgcctccaagccaagaggaaatgaccaagaaccaggtgtccctgacctgcctggtcaagggcttctacccttccgatatcgccgtggaatgggagtccaatggccagcctgagaacaactacaagaccacacctccagtgctggactccgacggctccttctttctgtactcccgcctgaccgtggacaagtccagatggcaagagggcaacgtgttctcctgctccgtgatgcacgaggccctgcacaatcactacacccagaagtccctgtctctgtccctgggctaa

SEQ ID NO: 83 - DNA, 1069 bp, synthetic construct
  misc_feature 1..1069: note = Fusion protein
  source 1..1069: mol_type = other DNA
ggggacaagtttgtacaaaaaagcaggctatggtaccgccaccatgaaatgggtcacctttatctccctgctgttcctgttctcctccgcctactcttgtgccgagcagaccacagagtggaccgcctgctctaagtcttgcggcatgggcttctccaccagagtgaccaaccggaacagacagtgcgagatgctgaagcagacccggctgtgtatggttcgaccttgcgaggccgctgccaaagaggctgctgctaaagaagccgccgcaaaagaggcagcagcaaaagaggctgccgccaaagaggccgcagccaaagaagcagcagctaaagaggccgctgcaaaagaacggaagtgctgcgtggaatgccctccttgtcctgctcctcctgtggctggcccttccgtgtttctgttccctccaaagcctaaggacaccctgatgatctctcggacccctgaagtgacctgcgtggtggtggatgtgtcccaagaggatcccgaggtgcagttcaattggtacgtggacggcgtggaagtgcacaacgccaagaccaagcctagagaggaacagttcaactccacctacagagtggtgtccgtgctgaccgtgctgcaccaggattggctgaacggcaaagagtacaagtgcaaggtgtccaacaagggcctgccttccagcatcgaaaagaccatctccaaggccaagggacagcccagagaaccccaggtgtacacactgcctccaagccaagaggaaatgaccaagaaccaggtgtccctgacctgcctggtcaagggcttctacccttccgatatcgccgtggaatgggagtccaatggccagcctgagaacaactacaagaccacacctccagtgctggactccgacggctccttctttctgtactcccgcctgaccgtggacaagtccagatggcaagagggcaacgtgttctcctgctccgtgatgcacgaggccctgcacaatcactacacccagaagtccctgtctctgtccctgggctaatctagaaacccagctttcttgtacaaagtggtcccc

SEQ ID NO: 84 - protein, 276 aa, synthetic construct
  REGION 1..276: note = Fusion protein
CAEQTTEWTACSKSCGMGFSTRVTNRNRQCEMLKQTRLCMVRPCEAAAKERKQQVEQPPQPAPPVAGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTFPPSQEEMTKNQVSLRCLVKGFYPSDIAVEWESNGQPENNYKTTKPVLDSDGSFRLESRLTVDKSRWQEGNVFSCSVMHEALHNHYTQKSLSLSLG

SEQ ID NO: 85 - protein, 294 aa, synthetic construct
  REGION 1..294: note = Fusion protein
MKWVTFISLLFLFSSAYSCAEQTTEWTACSKSCGMGFSTRVTNRNRQCEMLKQTRLCMVRPCEAAAKERKQQVEQPPQPAPPVAGPSVFLFPPKPKDTLYITREPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTFPPSQEEMTKNQVSLRCLVKGFYPSDIAVEWESNGQPENNYKTTKPVLDSDGSFRLESRLTVDKSRWQEGNVFSCSVMHEALHNHYTQKSLSLSLG

SEQ ID NO: 86 - DNA, 891 bp, synthetic construct
  misc_feature 1..891: note = Fusion protein
  source 1..891: mol_type = other DNA
gccaccatgaaatgggtcacctttatctccctgctgttcctgttctcctccgcctactcttgtgccgagcagaccacagagtggaccgcctgctctaagtcttgcggcatgggcttctccaccagagtgaccaaccggaacagacagtgcgagatgctgaagcagacccggctgtgtatggttcgaccttgcgaggccgctgccaaagaaagaaagcagcaggtcgagcagcctcctcagcctgctcctcctgttgctggcccttccgtgtttctgttccctccaaagcctaaggacaccctgtacatcacccgcgagcctgaagtgacctgcgtggtggtggatgtgtcccaagaggatcccgaggtgcagttcaattggtacgtggacggcgtggaagtgcacaacgccaagaccaagcctagagaggaacagttcaactccacctacagagtggtgtccgtgctgaccgtgctgcaccaggattggctgaacggcaaagagtacaagtgcaaggtgtccaacaagggcctgccttccagcatcgaaaagaccatctccaaggccaagggacagcccagagaaccccaggtgtacacattccctccatctcaagaggaaatgaccaagaaccaggtgtccctgcggtgcctggtcaagggcttctacccttctgatatcgccgtggaatgggagtccaacggccagcctgagaacaactacaagaccaccaagcctgtgctggactccgacggctccttccggcttgaatctagactgaccgtggacaagtcccggtggcaagagggcaacgtgttctcctgctctgtgatgcacgaggccctgcacaaccactacacccagaagtccctgtctctgtccctgggctaa

SEQ ID NO: 87 - DNA, 964 bp, synthetic construct
  misc_feature 1..964: note = Fusion protein
  source 1..964: mol_type = other DNA
ggggacaagtttgtacaaaaaagcaggctatggtaccgccaccatgaaatgggtcacctttatctccctgctgttcctgttctcctccgcctactcttgtgccgagcagaccacagagtggaccgcctgctctaagtcttgcggcatgggcttctccaccagagtgaccaaccggaacagacagtgcgagatgctgaagcagacccggctgtgtatggttcgaccttgcgaggccgctgccaaagaaagaaagcagcaggtcgagcagcctcctcagcctgctcctcctgttgctggcccttccgtgtttctgttccctccaaagcctaaggacaccctgtacatcacccgcgagcctgaagtgacctgcgtggtggtggatgtgtcccaagaggatcccgaggtgcagttcaattggtacgtggacggcgtggaagtgcacaacgccaagaccaagcctagagaggaacagttcaactccacctacagagtggtgtccgtgctgaccgtgctgcaccaggattggctgaacggcaaagagtacaagtgcaaggtgtccaacaagggcctgccttccagcatcgaaaagaccatctccaaggccaagggacagcccagagaaccccaggtgtacacattccctccatctcaagaggaaatgaccaagaaccaggtgtccctgcggtgcctggtcaagggcttctacccttctgatatcgccgtggaatgggagtccaacggccagcctgagaacaactacaagaccaccaagcctgtgctggactccgacggctccttccggcttgaatctagactgaccgtggacaagtcccggtggcaagagggcaacgtgttctcctgctctgtgatgcacgaggccctgcacaaccactacacccagaagtccctgtctctgtccctgggctaatctagaaacccagctttcttgtacaaagtggtcccc

SEQ ID NO: 88 - protein, 281 aa, synthetic construct
  REGION 1..281: note = Fusion protein
CAEQTTEWTACSKSCGMGFSTRVTNRNRQCEMLKQTRLCMVRPCEAAAKEPKSQDKTHTQPPQPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSRDELTKNQVSLRCHVKGFYPSDIAVEWESNGQPENNYKTTKPVLDSDGSFFLYSTLTVDKSRWQQGNVFSCSVLHEALHNHYTQKSLSLSPGK

SEQ ID NO: 89 - protein, 299 aa, synthetic construct
  REGION 1..299: note = Fusion protein
MKWVTFISLLFLFSSAYSCAEQTTEWTACSKSCGMGFSTRVTNRNRQCEMLKQTRLCMVRPCEAAAKEPKSQDKTHTQPPQPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSRDELTKNQVSLRCHVKGFYPSDIAVEWESNGQPENNYKTTKPVLDSDGSFFLYSTLTVDKSRWQQGNVFSCSVLHEALHNHYTQKSLSLSPGK

SEQ ID NO: 90 - DNA, 906 bp, synthetic construct
  misc_feature 1..906: note = Fusion protein
  source 1..906: mol_type = other DNA
gccaccatgaaatgggtcacctttatctccctgctgttcctgttctcctccgcctactcttgtgccgagcagaccacagagtggaccgcctgctctaagtcttgcggcatgggcttctccaccagagtgaccaaccggaacagacagtgcgagatgctgaagcagacccggctgtgtatggttcgaccttgcgaggccgctgccaaagagcctaagagccaggacaagacccacacacagcctccacagcctgctccagaattgctcggaggcccttccgtgtttctgttccctccaaagcctaaggacaccctgatgatctctcggacccctgaagtgacctgcgtggtggtggatgtgtctcacgaggatcccgaagtgaagttcaattggtacgtggacggcgtggaagtgcacaacgccaagacaaagccctgcgaggaacagtacggctccacctacagatgcgtgtccgtgctgacagtgctgcaccaggattggctgaacggcaaagagtacaagtgcaaggtgtccaacaaggccctgcctgctcctatcgaaaagaccatctccaaggccaagggccagcctagagaaccccaggtgtacacactgccaccttctagggacgagctgaccaagaaccaggtgtccctgagatgccacgtgaagggcttctacccctccgatatcgccgtggaatgggagtctaatggacagcccgagaacaactacaagaccaccaagcctgtgctggactccgacggctccttcttcctgtactctaccctgaccgtggacaagtccagatggcagcagggcaacgtgttctcctgctctgtgctgcacgaggccctgcacaatcactacacccagaagtccctgtctctgtcccctggcaagtga

SEQ ID NO: 91 - DNA, 979 bp, synthetic construct
  misc_feature 1..979: note = Fusion protein
  source 1..979: mol_type = other DNA
ggggacaagtttgtacaaaaaagcaggctatggtaccgccaccatgaaatgggtcacctttatctccctgctgttcctgttctcctccgcctactcttgtgccgagcagaccacagagtggaccgcctgctctaagtcttgcggcatgggcttctccaccagagtgaccaaccggaacagacagtgcgagatgctgaagcagacccggctgtgtatggttcgaccttgcgaggccgctgccaaagagcctaagagccaggacaagacccacacacagcctccacagcctgctccagaattgctcggaggcccttccgtgtttctgttccctccaaagcctaaggacaccctgatgatctctcggacccctgaagtgacctgcgtggtggtggatgtgtctcacgaggatcccgaagtgaagttcaattggtacgtggacggcgtggaagtgcacaacgccaagacaaagccctgcgaggaacagtacggctccacctacagatgcgtgtccgtgctgacagtgctgcaccaggattggctgaacggcaaagagtacaagtgcaaggtgtccaacaaggccctgcctgctcctatcgaaaagaccatctccaaggccaagggccagcctagagaaccccaggtgtacacactgccaccttctagggacgagctgaccaagaaccaggtgtccctgagatgccacgtgaagggcttctacccctccgatatcgccgtggaatgggagtctaatggacagcccgagaacaactacaagaccaccaagcctgtgctggactccgacggctccttcttcctgtactctaccctgaccgtggacaagtccagatggcagcagggcaacgtgttctcctgctctgtgctgcacgaggccctgcacaatcactacacccagaagtccctgtctctgtcccctggcaagtgatctagaaacccagctttcttgtacaaagtggtcccc

SEQ ID NO: 92 - protein, 402 aa, synthetic construct
  REGION 1..402: note = Fusion tag
GHHHHHHGSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGLQDSEVNQEAKPEVKPEVKPETHINLKVSDGSSEIFFKIKKTTPLRRLMEAFAKRQGKEMDSLTFLYDGIEIQADQTPEDLDMEDNDIIEAHREQIGG

SEQ ID NO: 93 - protein, 20 aa, synthetic construct
  REGION 1..20: note = Linker
SGGSGGSGGSGGSSGGGSSG

SEQ ID NO: 94 - protein, 466 aa, synthetic construct
  REGION 1..466: note = Fusion protein
GHHHHHHGSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGLQDSEVNQEAKPEVKPEVKPETHINLKVSDGSSEIFFKIKKTTPLRRLMEAFAKRQGKEMDSLTFLYDGIEIQADQTPEDLDMEDNDIIEAHREQIGGSGGSGGSGGSGGSSGGGSSGCAEQTTEWTACSKSCGMGFSTRVTNRNRQCEMLKQTRLCMVRPC

SEQ ID NO: 95 - DNA, 1461 bp, synthetic construct
  misc_feature 1..1461: note = Fusion protein
  source 1..1461: mol_type = other DNA
gccaccatgaaatgggtcacctttatctccctgctgttcctgttctcctccgcctactctggccaccaccatcaccatcacggctccgagatcggaaccggctttcctttcgaccctcactacgtggaagtgctgggcgagagaatgcactatgtggacgtgggccccagagatggaacccctgtgctgtttctgcacggcaaccctacctccagctacgtgtggcggaacatcatccctcacgtggcccctacacacagatgtatcgcccctgacctgatcggcatgggcaagtctgacaagcctgacctgggctacttcttcgacgaccacgtgcggttcatggacgcctttatcgaggctctgggcctcgaagaggtggtgctggtcatccatgattggggctctgccctgggctttcactgggccaagagaaaccccgagagagtgaagggaatcgccttcatggaattcatccggcctattcctacctgggacgagtggcctgagttcgccagagagacattccaggccttcagaaccaccgacgtgggcagaaagctgatcatcgaccagaacgtgttcatcgagggcaccctgcctatgggagtcgtcagacctctgaccgaggtggaaatggaccactacagagagccctttctgaaccccgtggaccgggaacctctttggagattccctaacgagctgcctatcgctggcgagcctgccaatattgtggccctggtggaagagtacatggactggctgcatcagagccccgtgcctaagctgctgttttggggaacacccggcgtgctgattcctcctgctgaagctgctagactggccaagagcctgcctaactgcaaggccgtggatatcggccctggcctgaatctgctgcaagaggacaaccccgatctgatcggatctgagatcgcccggtggctgagcaccctggaaatcagtggactgcaggactccgaagtgaatcaagaggccaagcctgaagtgaagcccgaagtcaagcctgagacacacatcaacctgaaggtgtccgacggctccagcgagatcttcttcaagatcaagaaaaccacacctctgcggcggctgatggaagcctttgccaagagacagggcaaagagatggactccctgaccttcctgtacgacggcatcgagatccaggccgatcagacccctgaggacctggacatggaagataacgacatcattgaggcccacagagagcagatcggcggctctggtggtagcggaggttctggtggatctggtggttcttctggcggcggatcttctggctgtgctgagcagacaaccgagtggaccgcctgctctaagtcttgtggcatgggcttctccaccagagtgaccaaccggaacagacagtgcgagatgctgaagcagacccggctgtgtatggtccgaccttgctaa

SEQ ID NO: 96 - DNA, 1552 bp, synthetic construct
  misc_feature 1..1552: note = Fusion protein
  source 1..1552: mol_type = other DNA
ggggacaagtttgtacaaaaaagcaggctataagcttgctgccaccatgaaatgggtcacctttatctccctgctgttcctgttctcctccgcctactctggccaccaccatcaccatcacggctccgagatcggaaccggctttcctttcgaccctcactacgtggaagtgctgggcgagagaatgcactatgtggacgtgggccccagagatggaacccctgtgctgtttctgcacggcaaccctacctccagctacgtgtggcggaacatcatccctcacgtggcccctacacacagatgtatcgcccctgacctgatcggcatgggcaagtctgacaagcctgacctgggctacttcttcgacgaccacgtgcggttcatggacgcctttatcgaggctctgggcctcgaagaggtggtgctggtcatccatgattggggctctgccctgggctttcactgggccaagagaaaccccgagagagtgaagggaatcgccttcatggaattcatccggcctattcctacctgggacgagtggcctgagttcgccagagagacattccaggccttcagaaccaccgacgtgggcagaaagctgatcatcgaccagaacgtgttcatcgagggcaccctgcctatgggagtcgtcagacctctgaccgaggtggaaatggaccactacagagagccctttctgaaccccgtggaccgggaacctctttggagattccctaacgagctgcctatcgctggcgagcctgccaatattgtggccctggtggaagagtacatggactggctgcatcagagccccgtgcctaagctgctgttttggggaacacccggcgtgctgattcctcctgctgaagctgctagactggccaagagcctgcctaactgcaaggccgtggatatcggccctggcctgaatctgctgcaagaggacaaccccgatctgatcggatctgagatcgcccggtggctgagcaccctggaaatcagtggactgcaggactccgaagtgaatcaagaggccaagcctgaagtgaagcccgaagtcaagcctgagacacacatcaacctgaaggtgtccgacggctccagcgagatcttcttcaagatcaagaaaaccacacctctgcggcggctgatggaagcctttgccaagagacagggcaaagagatggactccctgaccttcctgtacgacggcatcgagatccaggccgatcagacccctgaggacctggacatggaagataacgacatcattgaggcccacagagagcagatcggcggctctggtggtagcggaggttctggtggatctggtggttcttctggcggcggatcttctggctgtgctgagcagacaaccgagtggaccgcctgctctaagtcttgtggcatgggcttctccaccagagtgaccaaccggaacagacagtgcgagatgctgaagcagacccggctgtgtatggtccgaccttgctaaatctagagcggccgcggtaccaacccagctttcttgtacaaagtggtcccc

SEQ ID NO: 97 - protein, 634 aa, synthetic construct
  REGION 1..634: note = Fusion protein
CAEWSTAWGPCSTTCGLGMATRVSNQNRFCRLETQRRLCLSRPCEAAAKDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 98 - protein, 652 aa, synthetic construct
  REGION 1..652: note = Fusion protein
MKWVTFISLLFLFSSAYSCAEWSTAWGPCSTTCGLGMATRVSNQNRFCRLETQRRLCLSRPCEAAAKDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 99 - DNA, 1965 bp, synthetic construct
  misc_feature 1..1965: note = Fusion protein
  source 1..1965: mol_type = other DNA
gccaccatgaaatgggtcacctttatctccctgctgttcctgttctcctccgcctactcttgcgccgagtggtctacagcttggggcccttgttctaccacctgtggcctcggcatggccaccagagtgtccaaccagaacagattctgccggctggaaacccagcggagactgtgcttgtctagaccttgcgaggccgctgccaaggacgctcataagtctgaggtggcccaccggttcaaggacctgggcgaagagaacttcaaggccctggtgctgatcgccttcgctcagtacttgcagcagtgccccttcgaggaccacgtgaagctggtcaacgaagtgaccgagttcgccaagacctgcgtggccgatgagtctgccgagaactgcgacaagtctctgcacaccctgttcggcgacaagctgtgtaccgtggctaccctgagagaaacctacggcgagatggccgactgctgcgctaagcaagagcccgagagaaacgagtgcttcctgcagcacaaggacgacaaccctaacctgcctagactcgtgcggcctgaggtggacgtgatgtgtaccgccttccacgacaacgaggaaaccttcctgaagaagtacctgtacgagatcgccagacggcacccctacttttacgcccctgagctgctgttcttcgccaagcggtacaaggccgccttcaccgagtgttgtcaggccgctgataaggccgcttgcctgctgcctaaactggacgagctgagagatgaaggcaaggcctccagcgccaagcagagactgaagtgtgccagcctgcagaagttcggcgagagagcctttaaggcctgggccgtcgctagactgtcccagagatttcccaaggccgagtttgccgaggtgtccaagctggttaccgacctgaccaaggtgcacaccgaatgctgtcacggcgacctgctggaatgcgccgatgatagagccgatctggccaagtacatctgcgagaaccaggactccatctcctccaagctgaaagagtgctgcgagaagcctctgctggaaaagtcccactgtatcgccgaggtggaaaacgacgagatgcctgccgatctgccttctctggccgccgacttcgtggaatctaaggacgtgtgcaagaactacgccgaggctaaggatgtgttcctgggcatgtttctgtacgagtacgctcggcggcaccccgactattctgttgtgctgctgctgagactggctaagacctacgagacaaccctcgagaagtgctgtgccgccgctgatcctcacgagtgttacgccaaggtgttcgacgagttcaagccactggtggaagaaccccagaacctgatcaagcagaattgcgagctgttcgagcagctgggcgagtacaagttccagaacgccctgctcgtgcggtacaccaagaaagtgccccaggtgtccacacctacactggttgaggtgtcccggaacctgggcaaagtgggctctaagtgctgcaagcaccccgaggccaagagaatgccttgtgccgaggactacctgtccgtggtgctgaaccagctgtgcgtgctgcacgaaaagacccctgtgtccgacagagtgaccaagtgctgtaccgagagcctggtcaacagacggccttgcttctctgccctggaagtggacgagacatacgtgcccaaagagttcaacgccgagacattcaccttccacgccgacatctgcaccctgtccgagaaagagcggcagatcaagaaacagaccgctctggtggaactggtcaagcacaagcccaaggccaccaaagaacagctgaaggccgtgatggacgacttcgccgcctttgtggaaaagtgttgcaaggccgacgacaaagagacatgcttcgccgaagagggcaagaaactggtggccgcttctcaggctgctctgggactttaa

SEQ ID NO: 100 - DNA, 2038 bp, synthetic construct
  misc_feature 1..2038: note = Fusion protein
  source 1..2038: mol_type = other DNA
ggggacaagtttgtacaaaaaagcaggctatggtaccgccaccatgaaatgggtcacctttatctccctgctgttcctgttctcctccgcctactcttgcgccgagtggtctacagcttggggcccttgttctaccacctgtggcctcggcatggccaccagagtgtccaaccagaacagattctgccggctggaaacccagcggagactgtgcttgtctagaccttgcgaggccgctgccaaggacgctcataagtctgaggtggcccaccggttcaaggacctgggcgaagagaacttcaaggccctggtgctgatcgccttcgctcagtacttgcagcagtgccccttcgaggaccacgtgaagctggtcaacgaagtgaccgagttcgccaagacctgcgtggccgatgagtctgccgagaactgcgacaagtctctgcacaccctgttcggcgacaagctgtgtaccgtggctaccctgagagaaacctacggcgagatggccgactgctgcgctaagcaagagcccgagagaaacgagtgcttcctgcagcacaaggacgacaaccctaacctgcctagactcgtgcggcctgaggtggacgtgatgtgtaccgccttccacgacaacgaggaaaccttcctgaagaagtacctgtacgagatcgccagacggcacccctacttttacgcccctgagctgctgttcttcgccaagcggtacaaggccgccttcaccgagtgttgtcaggccgctgataaggccgcttgcctgctgcctaaactggacgagctgagagatgaaggcaaggcctccagcgccaagcagagactgaagtgtgccagcctgcagaagttcggcgagagagcctttaaggcctgggccgtcgctagactgtcccagagatttcccaaggccgagtttgccgaggtgtccaagctggttaccgacctgaccaaggtgcacaccgaatgctgtcacggcgacctgctggaatgcgccgatgatagagccgatctggccaagtacatctgcgagaaccaggactccatctcctccaagctgaaagagtgctgcgagaagcctctgctggaaaagtcccactgtatcgccgaggtggaaaacgacgagatgcctgccgatctgccttctctggccgccgacttcgtggaatctaaggacgtgtgcaagaactacgccgaggctaaggatgtgttcctgggcatgtttctgtacgagtacgctcggcggcaccccgactattctgttgtgctgctgctgagactggctaagacctacgagacaaccctcgagaagtgctgtgccgccgctgatcctcacgagtgttacgccaaggtgttcgacgagttcaagccactggtggaagaaccccagaacctgatcaagcagaattgcgagctgttcgagcagctgggcgagtacaagttccagaacgccctgctcgtgcggtacaccaagaaagtgccccaggtgtccacacctacactggttgaggtgtcccggaacctgggcaaagtgggctctaagtgctgcaagcaccccgaggccaagagaatgccttgtgccgaggactacctgtccgtggtgctgaaccagctgtgcgtgctgcacgaaaagacccctgtgtccgacagagtgaccaagtgctgtaccgagagcctggtcaacagacggccttgcttctctgccctggaagtggacgagacatacgtgcccaaagagttcaacgccgagacattcaccttccacgccgacatctgcaccctgtccgagaaagagcggcagatcaagaaacagaccgctctggtggaactggtcaagcacaagcccaaggccaccaaagaacagctgaaggccgtgatggacgacttcgccgcctttgtggaaaagtgttgcaaggccgacgacaaagagacatgcttcgccgaagagggcaagaaactggtggccgcttctcaggctgctctgggactttaatctagaaacccagctttcttgtacaaagtggtcccc

SEQ ID NO: 101 - protein, 582 aa, Homo sapiens
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAA

SEQ ID NO: 102 - protein, 675 aa, synthetic construct
  REGION 1..675: note = Fusion protein
DAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAAEAAAKEAAAKEAAAKEAAAKEAAAKEAAAKEAAAKEAAAKCAEWSTAWGPCSTTCGLGMATRVSNQNRFCRLETQRRLCLSRPCPPSRGRSPQ

SEQ ID NO: 103 - protein, 693 aa, synthetic construct
  REGION 1..693: note = Fusion protein
MKWVTFISLLFLFSSAYSDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAAEAAAKEAAAKEAAAKEAAAKEAAAKEAAAKEAAAKEAAAKCAEWSTAWGPCSTTCGLGMATRVSNQNRFCRLETQRRLCLSRPCPPSRGRSPQ

SEQ ID NO: 104 - DNA, 2088 bp, synthetic construct
  misc_feature 1..2088: note = Fusion protein
  source 1..2088: mol_type = other DNA
gccaccatgaaatgggtcacctttatctccctgctgttcctgttctcctccgcctactctgacgcccacaagtctgaggtggcccacagattcaaggacctgggcgaagagaacttcaaggccctggtgctgatcgccttcgctcagtacttgcagcagtgccccttcgaggaccacgtgaagctggtcaacgaagtgaccgagttcgccaagacctgcgtggccgatgagtctgccgagaactgcgacaagtctctgcacaccctgttcggcgacaagctgtgtaccgtggctaccctgagagaaacctacggcgagatggccgactgctgcgctaagcaagagcccgagagaaacgagtgcttcctgcagcacaaggacgacaaccctaacctgcctagactcgtgcggcctgaggtggacgtgatgtgtaccgccttccacgacaacgaggaaaccttcctgaagaagtacctgtacgagatcgccagacggcacccctacttttacgcccctgagctgctgttcttcgccaagcggtacaaggccgccttcaccgagtgttgtcaggccgctgataaggccgcttgcctgctgcctaaactggacgagctgagagatgaaggcaaggcctccagcgccaagcagagactgaagtgtgccagcctgcagaagttcggcgagagagcctttaaggcctgggccgtcgctagactgtcccagagatttcccaaggccgagtttgccgaggtgtccaagctggttaccgacctgaccaaggtgcacaccgaatgctgtcacggcgacctgctggaatgcgccgatgatagagccgatctggccaagtacatctgcgagaaccaggactccatctcctccaagctgaaagagtgctgcgagaagcctctgctggaaaagtcccactgtatcgccgaggtggaaaacgacgagatgcctgccgatctgccttctctggccgccgacttcgtggaatctaaggacgtgtgcaagaactacgccgaggccaaggatgtgttcctgggcatgtttctgtacgagtacgctcggcggcaccccgactattctgttgtgctgctgctgagactggctaagacctacgagacaaccctcgagaagtgctgtgccgccgctgatcctcacgagtgttacgccaaggtgttcgacgagttcaagccactggtggaagaaccccagaacctgatcaagcagaattgcgagctgttcgagcagctgggcgagtacaagttccagaacgccctgctcgtgcggtacaccaagaaagtgccccaggtgtccacacctacactggttgaggtgtcccggaacctgggcaaagtgggctctaagtgctgcaagcaccctgaggccaagagaatgccttgcgccgaggactacctgtccgtggtgctgaatcagctgtgcgtgctgcacgaaaagacccctgtgtccgacagagtgaccaagtgctgtaccgagagcctggtcaacagacggccttgcttctctgccctggaagtggacgagacatacgtgcccaaagagttcaacgccgagacattcaccttccacgccgacatctgcaccctgtccgagaaagagcggcagatcaagaaacagaccgctctggtggaactggtcaagcacaagcccaaggccaccaaagaacagctgaaggccgtgatggacgacttcgccgcctttgtggaaaagtgttgcaaggccgacgacaaagagacatgcttcgccgaagagggcaagaaactggtggccgcttctcaggctgctgaggccgctgctaaagaggctgccgctaaagaagccgcagccaaagaggcagctgcaaaagaagctgctgcaaaagaggcagccgccaaagaggccgctgctaaagaagcagccgccaagtgtgctgagtggtctacagcttggggcccctgctctacaacctgtggactcggcatggccaccagagtgtctaaccagaacagattctgccggctggaaacccagcggagactgtgcctgtctagaccctgtcctcctagcagaggcagatcccctcagtga

SEQ ID NO: 105 - DNA, 2161 bp, synthetic construct
  misc_feature 1..2161: note = Fusion protein
  source 1..2161: mol_type = other DNA
ggggacaagtttgtacaaaaaagcaggctatggtaccgccaccatgaaatgggtcacctttatctccctgctgttcctgttctcctccgcctactctgacgcccacaagtctgaggtggcccacagattcaaggacctgggcgaagagaacttcaaggccctggtgctgatcgccttcgctcagtacttgcagcagtgccccttcgaggaccacgtgaagctggtcaacgaagtgaccgagttcgccaagacctgcgtggccgatgagtctgccgagaactgcgacaagtctctgcacaccctgttcggcgacaagctgtgtaccgtggctaccctgagagaaacctacggcgagatggccgactgctgcgctaagcaagagcccgagagaaacgagtgcttcctgcagcacaaggacgacaaccctaacctgcctagactcgtgcggcctgaggtggacgtgatgtgtaccgccttccacgacaacgaggaaaccttcctgaagaagtacctgtacgagatcgccagacggcacccctacttttacgcccctgagctgctgttcttcgccaagcggtacaaggccgccttcaccgagtgttgtcaggccgctgataaggccgcttgcctgctgcctaaactggacgagctgagagatgaaggcaaggcctccagcgccaagcagagactgaagtgtgccagcctgcagaagttcggcgagagagcctttaaggcctgggccgtcgctagactgtcccagagatttcccaaggccgagtttgccgaggtgtccaagctggttaccgacctgaccaaggtgcacaccgaatgctgtcacggcgacctgctggaatgcgccgatgatagagccgatctggccaagtacatctgcgagaaccaggactccatctcctccaagctgaaagagtgctgcgagaagcctctgctggaaaagtcccactgtatcgccgaggtggaaaacgacgagatgcctgccgatctgccttctctggccgccgacttcgtggaatctaaggacgtgtgcaagaactacgccgaggccaaggatgtgttcctgggcatgtttctgtacgagtacgctcggcggcaccccgactattctgttgtgctgctgctgagactggctaagacctacgagacaaccctcgagaagtgctgtgccgccgctgatcctcacgagtgttacgccaaggtgttcgacgagttcaagccactggtggaagaaccccagaacctgatcaagcagaattgcgagctgttcgagcagctgggcgagtacaagttccagaacgccctgctcgtgcggtacaccaagaaagtgccccaggtgtccacacctacactggttgaggtgtcccggaacctgggcaaagtgggctctaagtgctgcaagcaccctgaggccaagagaatgccttgcgccgaggactacctgtccgtggtgctgaatcagctgtgcgtgctgcacgaaaagacccctgtgtccgacagagtgaccaagtgctgtaccgagagcctggtcaacagacggccttgcttctctgccctggaagtggacgagacatacgtgcccaaagagttcaacgccgagacattcaccttccacgccgacatctgcaccctgtccgagaaagagcggcagatcaagaaacagaccgctctggtggaactggtcaagcacaagcccaaggccaccaaagaacagctgaaggccgtgatggacgacttcgccgcctttgtggaaaagtgttgcaaggccgacgacaaagagacatgcttcgccgaagagggcaagaaactggtggccgcttctcaggctgctgaggccgctgctaaagaggctgccgctaaagaagccgcagccaaagaggcagctgcaaaagaagctgctgcaaaagaggcagccgccaaagaggccgctgctaaagaagcagccgccaagtgtgctgagtggtctacagcttggggcccctgctctacaacctgtggactcggcatggccaccagagtgtctaaccagaacagattctgccggctggaaacccagcggagactgtgcctgtctagaccctgtcctcctagcagaggcagatcccctcagtgatctagaaacccagctttcttgtacaaagtggtcccc

SEQ ID NO: 106 - protein, 634 aa, synthetic construct
  REGION 1..634: note = Fusion protein
CAEQTTEWTACSKSCGMGFSTRVTNRNRQCEMLKQTRLCMVRPCEAAAKDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 107 - protein, 652 aa, synthetic construct
  REGION 1..652: note = Fusion protein
MKWVTFISLLFLFSSAYSCAEQTTEWTACSKSCGMGFSTRVTNRNRQCEMLKQTRLCMVRPCEAAAKDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 108 - DNA, 1965 bp, synthetic construct
  misc_feature 1..1965: note = Fusion protein
  source 1..1965: mol_type = other DNA
gccaccatgaaatgggtcacctttatctccctgctgttcctgttctcctccgcctactcttgtgccgagcagaccacagagtggaccgcctgctctaagtcttgcggcatgggcttctccaccagagtgaccaaccggaacagacagtgcgagatgctgaagcagacccggctgtgtatggttcgaccttgcgaggccgctgccaaggatgctcataagtctgaggtggcccaccggttcaaggacctgggcgaagagaacttcaaggccctggtgctgatcgccttcgctcagtacttgcagcagtgccccttcgaggaccacgtgaagctggtcaacgaagtgaccgagttcgccaagacctgcgtggccgatgagtctgccgagaactgcgacaagtctctgcacaccctgttcggcgacaagctgtgtaccgtggctaccctgagagaaacctacggcgagatggccgactgctgcgctaagcaagagcccgagagaaacgagtgcttcctgcagcacaaggacgacaaccctaacctgcctagactcgtgcggcctgaggtggacgtgatgtgtaccgccttccacgacaacgaggaaaccttcctgaagaagtacctgtacgagatcgccagacggcacccctacttttacgcccctgagctgctgttcttcgccaagcggtacaaggccgccttcaccgagtgttgtcaggccgctgataaggccgcttgcctgctgcctaaactggacgagctgagagatgaaggcaaggcctccagcgccaagcagagactgaagtgtgccagcctgcagaagttcggcgagagagcctttaaggcctgggccgtcgctagactgtcccagagatttcccaaggccgagtttgccgaggtgtccaagctggttaccgacctgaccaaggtgcacaccgaatgctgtcacggcgacctgctggaatgcgccgatgatagagccgatctggccaagtacatctgcgagaaccaggactccatctcctccaagctgaaagagtgctgcgagaagcctctgctggaaaagtcccactgtatcgccgaggtggaaaacgacgagatgcctgccgatctgccttctctggccgccgacttcgtggaatctaaggacgtgtgcaagaactacgccgaggctaaggatgtgttcctgggcatgtttctgtacgagtacgctcggcggcaccccgattatagtgtggtgctgctgctgagactggctaagacctacgagacaaccctcgagaagtgctgtgccgccgctgatcctcacgagtgttacgccaaggtgttcgacgagttcaagccactggtggaagaaccccagaacctgatcaagcagaattgcgagctgttcgagcagctgggcgagtacaagttccagaacgccctgctcgtgcggtacaccaagaaagtgccccaggtgtccacacctacactggttgaggtgtcccggaacctgggcaaagtgggctctaagtgctgcaagcaccccgaggccaagagaatgccttgcgccgaggattacctgtccgtggtgctgaaccagctgtgcgtgctgcacgaaaagacccctgtgtccgaccgcgtgaccaagtgctgtacagagtccctggtcaacagacggccctgcttctctgccctggaagtggacgagacatacgtgcccaaagagttcaacgccgagacattcaccttccacgccgacatctgcaccctgtccgagaaagagcggcagatcaagaaacagaccgctctggtcgaactggtcaagcacaagcccaaggccaccaaagaacagctgaaggccgtgatggacgacttcgccgcctttgtggaaaagtgttgcaaggccgacgacaaagagacatgcttcgccgaagagggcaagaaactggtggccgcttctcaggctgctctgggactttaa

SEQ ID NO: 109 - DNA, 2038 bp, synthetic construct
  misc_feature 1..2038: note = Fusion protein
  source 1..2038: mol_type = other DNA
ggggacaagtttgtacaaaaaagcaggctatggtaccgccaccatgaaatgggtcacctttatctccctgctgttcctgttctcctccgcctactcttgtgccgagcagaccacagagtggaccgcctgctctaagtcttgcggcatgggcttctccaccagagtgaccaaccggaacagacagtgcgagatgctgaagcagacccggctgtgtatggttcgaccttgcgaggccgctgccaaggatgctcataagtctgaggtggcccaccggttcaaggacctgggcgaagagaacttcaaggccctggtgctgatcgccttcgctcagtacttgcagcagtgccccttcgaggaccacgtgaagctggtcaacgaagtgaccgagttcgccaagacctgcgtggccgatgagtctgccgagaactgcgacaagtctctgcacaccctgttcggcgacaagctgtgtaccgtggctaccctgagagaaacctacggcgagatggccgactgctgcgctaagcaagagcccgagagaaacgagtgcttcctgcagcacaaggacgacaaccctaacctgcctagactcgtgcggcctgaggtggacgtgatgtgtaccgccttccacgacaacgaggaaaccttcctgaagaagtacctgtacgagatcgccagacggcacccctacttttacgcccctgagctgctgttcttcgccaagcggtacaaggccgccttcaccgagtgttgtcaggccgctgataaggccgcttgcctgctgcctaaactggacgagctgagagatgaaggcaaggcctccagcgccaagcagagactgaagtgtgccagcctgcagaagttcggcgagagagcctttaaggcctgggccgtcgctagactgtcccagagatttcccaaggccgagtttgccgaggtgtccaagctggttaccgacctgaccaaggtgcacaccgaatgctgtcacggcgacctgctggaatgcgccgatgatagagccgatctggccaagtacatctgcgagaaccaggactccatctcctccaagctgaaagagtgctgcgagaagcctctgctggaaaagtcccactgtatcgccgaggtggaaaacgacgagatgcctgccgatctgccttctctggccgccgacttcgtggaatctaaggacgtgtgcaagaactacgccgaggctaaggatgtgttcctgggcatgtttctgtacgagtacgctcggcggcaccccgattatagtgtggtgctgctgctgagactggctaagacctacgagacaaccctcgagaagtgctgtgccgccgctgatcctcacgagtgttacgccaaggtgttcgacgagttcaagccactggtggaagaaccccagaacctgatcaagcagaattgcgagctgttcgagcagctgggcgagtacaagttccagaacgccctgctcgtgcggtacaccaagaaagtgccccaggtgtccacacctacactggttgaggtgtcccggaacctgggcaaagtgggctctaagtgctgcaagcaccccgaggccaagagaatgccttgcgccgaggattacctgtccgtggtgctgaaccagctgtgcgtgctgcacgaaaagacccctgtgtccgaccgcgtgaccaagtgctgtacagagtccctggtcaacagacggccctgcttctctgccctggaagtggacgagacatacgtgcccaaagagttcaacgccgagacattcaccttccacgccgacatctgcaccctgtccgagaaagagcggcagatcaagaaacagaccgctctggtcgaactggtcaagcacaagcccaaggccaccaaagaacagctgaaggccgtgatggacgacttcgccgcctttgtggaaaagtgttgcaaggccgacgacaaagagacatgcttcgccgaagagggcaagaaactggtggccgcttctcaggctgctctgggactttaatctagaaacccagctttcttgtacaaagtggtcccc

SEQ ID NO: 110 - protein, 669 aa, synthetic construct
  REGION 1..669: note = Fusion protein
CAEQTTEWTACSKSCGMGFSTRVTNRNRQCEMLKQTRLCMVRPCEAAAKEAAAKEAAAKEAAAKEAAAKEAAAKEAAAKEAAAKDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 111 - protein, 687 aa, synthetic construct
  REGION 1..687: note = Fusion protein
MKWVTFISLLFLFSSAYSCAEQTTEWTACSKSCGMGFSTRVTNRNRQCEMLKQTRLCMVRPCEAAAKEAAAKEAAAKEAAAKEAAAKEAAAKEAAAKEAAAKDAHKSEVAHRFKDLGEENFKALVLIAFAQYLQQCPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 112 - DNA, 2070 bp, synthetic construct
  misc_feature 1..2070: note = Fusion protein
  source 1..2070: mol_type = other DNA
gccaccatgaaatgggtcacctttatctccctgctgttcctgttctcctccgcctactcttgtgccgagcagaccacagagtggaccgcctgctctaagtcttgcggcatgggcttctccaccagagtgaccaaccggaacagacagtgcgagatgctgaagcagacccggctgtgtatggttcgaccttgcgaggccgctgccaaagaggctgctgctaaagaagccgccgcaaaagaggcagcagcaaaagaggctgccgccaaagaggccgcagccaaagaagcagcagctaaagaggccgctgctaaggacgcccacaagtctgaagtggcccaccggtttaaggacctgggcgaagagaacttcaaggccctggtgctgatcgccttcgctcagtacttgcagcagtgccccttcgaggaccacgtgaagctggtcaacgaagtgaccgagttcgccaagacctgcgtggccgatgagtctgccgagaactgcgacaagtctctgcacaccctgttcggcgacaagctgtgtaccgtggctaccctgagagaaacctacggcgagatggccgactgctgcgctaagcaagagcccgagagaaacgagtgcttcctgcagcacaaggacgacaaccctaacctgcctagactcgtgcggcctgaggtggacgtgatgtgtaccgccttccacgacaacgaggaaaccttcctgaagaagtacctgtacgagatcgccagacggcacccctacttttacgcccctgagctgctgtttttcgccaagcggtacaaggccgccttcaccgagtgttgtcaggccgccgataaggccgcttgtctgctgcctaaactggacgagctgcgcgacgaaggcaaggcctcttctgctaagcagcggctgaagtgcgccagcctgcagaagtttggcgagagagccttcaaggcttgggccgtcgctagactgtcccagagatttcccaaggccgagtttgccgaggtgtccaagctggttaccgacctgaccaaggtgcacaccgaatgctgtcacggcgacctgctggaatgcgccgatgatagagccgatctggccaagtacatctgcgagaaccaggactccatctcctccaagctgaaagagtgctgcgagaagcctctgctggaaaagtcccactgtatcgccgaggtggaaaacgacgagatgcctgccgatctgccttctctggccgccgacttcgtggaatctaaggacgtgtgcaagaactacgccgaggccaaggatgtgttcctgggcatgtttctgtacgagtacgctcggcggcaccccgattatagtgtggtgctgctgctgagactggctaagacctacgagacaaccctcgagaagtgctgtgccgccgctgatcctcacgagtgttacgccaaggtgttcgacgagttcaagccactggtggaagaaccccagaacctgatcaagcagaattgcgagctgttcgagcagctgggcgagtacaagttccagaacgccctgctcgtgcggtacaccaagaaagtgccccaggtgtccacacctacactggttgaggtgtcccggaacctgggcaaagtgggctctaagtgctgcaagcaccctgaggccaagagaatgccttgcgccgaggactacctgtccgtggtgctgaatcagctgtgcgtgctgcacgaaaagacccctgtgtccgaccgcgtgaccaagtgctgtacagagtccctggtcaacagacggccctgcttctctgccctggaagtggacgagacatacgtgcccaaagagttcaacgccgagacattcaccttccacgccgacatctgcaccctgtccgagaaagagcggcagatcaagaaacagaccgctctggtcgagctggttaagcacaagcccaaggccaccaaagaacagctgaaggccgtgatggacgacttcgccgcctttgtggaaaagtgttgcaaggccgacgacaaagagacatgcttcgccgaagagggcaagaaactggtggccgcttctcaggctgctctgggactttaa

SEQ ID NO: 113 - DNA, 2143 bp, synthetic construct
  misc_feature 1..2143: note = Fusion protein
  source 1..2143: mol_type = other DNA
ggggacaagtttgtacaaaaaagcaggctatggtaccgccaccatgaaatgggtcacctttatctccctgctgttcctgttctcctccgcctactcttgtgccgagcagaccacagagtggaccgcctgctctaagtcttgcggcatgggcttctccaccagagtgaccaaccggaacagacagtgcgagatgctgaagcagacccggctgtgtatggttcgaccttgcgaggccgctgccaaagaggctgctgctaaagaagccgccgcaaaagaggcagcagcaaaagaggctgccgccaaagaggccgcagccaaagaagcagcagctaaagaggccgctgctaaggacgcccacaagtctgaagtggcccaccggtttaaggacctgggcgaagagaacttcaaggccctggtgctgatcgccttcgctcagtacttgcagcagtgccccttcgaggaccacgtgaagctggtcaacgaagtgaccgagttcgccaagacctgcgtggccgatgagtctgccgagaactgcgacaagtctctgcacaccctgttcggcgacaagctgtgtaccgtggctaccctgagagaaacctacggcgagatggccgactgctgcgctaagcaagagcccgagagaaacgagtgcttcctgcagcacaaggacgacaaccctaacctgcctagactcgtgcggcctgaggtggacgtgatgtgtaccgccttccacgacaacgaggaaaccttcctgaagaagtacctgtacgagatcgccagacggcacccctacttttacgcccctgagctgctgtttttcgccaagcggtacaaggccgccttcaccgagtgttgtcaggccgccgataaggccgcttgtctgctgcctaaactggacgagctgcgcgacgaaggcaaggcctcttctgctaagcagcggctgaagtgcgccagcctgcagaagtttggcgagagagccttcaaggcttgggccgtcgctagactgtcccagagatttcccaaggccgagtttgccgaggtgtccaagctggttaccgacctgaccaaggtgcacaccgaatgctgtcacggcgacctgctggaatgcgccgatgatagagccgatctggccaagtacatctgcgagaaccaggactccatctcctccaagctgaaagagtgctgcgagaagcctctgctggaaaagtcccactgtatcgccgaggtggaaaacgacgagatgcctgccgatctgccttctctggccgccgacttcgtggaatctaaggacgtgtgcaagaactacgccgaggccaaggatgtgttcctgggcatgtttctgtacgagtacgctcggcggcaccccgattatagtgtggtgctgctgctgagactggctaagacctacgagacaaccctcgagaagtgctgtgccgccgctgatcctcacgagtgttacgccaaggtgttcgacgagttcaagccactggtggaagaaccccagaacctgatcaagcagaattgcgagctgttcgagcagctgggcgagtacaagttccagaacgccctgctcgtgcggtacaccaagaaagtgccccaggtgtccacacctacactggttgaggtgtcccggaacctgggcaaagtgggctctaagtgctgcaagcaccctgaggccaagagaatgccttgcgccgaggactacctgtccgtggtgctgaatcagctgtgcgtgctgcacgaaaagacccctgtgtccgaccgcgtgaccaagtgctgtacagagtccctggtcaacagacggccctgcttctctgccctggaagtggacgagacatacgtgcccaaagagttcaacgccgagacattcaccttccacgccgacatctgcaccctgtccgagaaagagcggcagatcaagaaacagaccgctctggtcgagctggttaagcacaagcccaaggccaccaaagaacagctgaaggccgtgatggacgacttcgccgcctttgtggaaaagtgttgcaaggccgacgacaaagagacatgcttcgccgaagagggcaagaaactggtggccgcttctcaggctgctctgggactttaatctagaaacccagctttcttgtacaaagtggtcccc

SEQ ID NO: 114 - protein, 484 aa, synthetic construct
  REGION 1..484: note = Fusion protein
MKWVTFISLLFLFSSAYSGHHHHHHGSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISGLQDSEVNQEAKPEVKPEVKPETHINLKVSDGSSEIFFKIKKTTPLRRLMEAFAKRQGKEMDSLTFLYDGIEIQADQTPEDLDMEDNDIIEAHREQIGGSGGSGGSGGSGGSSGGGSSGCAEQTTEWTACSKSCGMGFSTRVTNRNRQCEMLKQTRLCMVRPC

SEQ ID NO: 115 - protein, 13 aa, synthetic construct
  REGION 1..13: note = part of recombinant protein
PPSRGRSPQNSAF

SEQ ID NO: 116 - protein, 8 aa, synthetic construct
  REGION 1..8: note = part of recombinant protein
GQPVYSSL

SEQ ID NO: 117 - protein, 7 aa, synthetic construct
  REGION 1..7: note = part of recombinant protein
EADLEEN

SEQ ID NO: 118 - protein, 9 aa, synthetic construct
  REGION 1..9: note = part of recombinant protein
EQQPEQPTD

SEQ ID NO: 119 - protein, 8 aa, synthetic construct
  REGION 1..8: note = part of recombinant protein
DVDIHTLI

SEQ ID NO: 120 - protein, 11 aa, synthetic construct
  REGION 1..11: note = part of recombinant protein
DSNILKTIKIP

SEQ ID NO: 121 - protein, 5 aa, synthetic construct
  REGION 1..5: note = Linker
GGGGS